SEQUENCE LISTING (56 sequences)

SEQ ID NO: 1 - protein, 3011 aa, Hepacivirus C
MSTNPKPQKKNKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSTGLYHVTNDCPNSSIVYEAADAILHTPGCVPCVREGNASRCWVAMTPTVATRDGKLPATQLRRHIDLLVGSATLCSALYVGDLCGSVFLVGQLFTFSPRRHWTTQGCNCSIYPGHITGHRMAWDMMMNWSPTTALVMAQLLRIPQAILDMIAGAHWGVLAGIAYFSMVGNWAKVLVVLLLFAGVDAETHVTGGSAGHTVSGFVSLLAPGAKQNVQLINTNGSWHLNSTALNCNDSLNTGWLAGLFYHHKFNSSGCPERLASCRPLTDFDQGWGPISYANGSGPDQRPYCWHYPPKPCGIVPAKSVCGPVYCFTPSPVVVGTTDRSGAPTYSWGENDTDVFVLNNTRPPLGNWFGCTWMNSTGFTKVCGAPPCVIGGAGNNTLHCPTDCFRKHPDATYSRCGSGPWITPRCLVDYPYRLWHYPCTINYTIFKIRMYVGGVEHRLEAACNWTRGERCDLEDRDRSELSPLLLTTTQWQVLPCSFTTLPALSTGLIHLHQNIVDVQYLYGVGSSIASWAIKWEYVVLLFLLLADARVCSCLWMMLLISQAEAALENLVILNAASLAGTHGLVSFLVFFCFAWYLKGKWVPGAVYTFYGMWPLLLLLLALPQRAYALDTEVAASCGGVVLVGLMALTLSPYYKRYISWCLWWLQYFLTRVEAQLHVWIPPLNVRGGRDAVILLMCAVHPTLVFDITKLLLAVFGPLWILQASLLKVPYFVRVQGLLRFCALARKMIGGHYVQMVIIKLGALTGTYVYNHLTPLRDWAHNGLRDLAVAVEPVVFSQMETKLITWGADTAACGDIINGLPVSARRGREILLGPADGMVSKGWRLLAPITAYAQQTRGLLGCIITSLTGRDKNQVEGEVQIVSTAAQTFLATCINGVCWTVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGIFRAAVCTRGVAKAVDFIPVENLETTMRSPVFTDNSSPPVVPQSFQVAHLHAPTGSGKSTKVPAAYAAQGYKVLVLNPSVAATLGFGAYMSKAHGIDPNIRTGVRTITTGSPITYSTYGKFLADGGCSGGAYDIIICDECHSTDATSILGIGTVLDQAETAGARLVVLATATPPGSVTVPHPNIEEVALSTTGEIPFYGKAIPLEVIKGGRHLIFCHSKKKCDELAAKLVALGINAVAYYRGLDVSVIPTSGDVVVVATDALMTGYTGDFDSVIDCNTCVTQTVDFSLDPTFTIETITLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENLPYLVAYQATVCARAQAPPPSWDQMWKCLIRLKPTLHGPTPLLYRLGAVQNEITLTHPVTKYIMTCMSADLEVVTSTWVLVGGVLAALAAYCLSTGCVVIVGRVVLSGKPAIIPDREVLYREFDEMEECSQHLPYIEQGMMLAEQFKQKALGLLQTASRQAEVIAPAVQTNWQKLETFWAKHMWNFISGIQYLAGLSTLPGNPAIASLMAFTAAVTSPLTTSQTLLFNILGGWVAAQLAAPGAATAFVGAGLAGAAIGSVGLGKVLIDILAGYGAGVAGALVAFKIMSGEVPSTEDLVNLLPAILSPGALVVGVVCAAILRRHVGPGEGAVQWMNRLIAFASRGNHVSPTHYVPESDAAARVTAILSSLTVTQLLRRLHQWISSECTTPCSGSWLRDIWDWICEVLSDFKTWLKAKLMPQLPGIPFVSCQRGYKGVWRVDGIMHTRCHCGAEITGHVKNGTMRIVGPRTCRNMWSGTFPINAYTTGPCTPLPAPNYTFALWRVSAEEYVEIRQVGDFHYVTGMTTDNLKCPCQVPSPEFFTELDGVRLHRFAPPCKPLLREEVSFRVGLHEYPVGSQLPCEPEPDVAVLTSMLTDPSHITAEAAGRRLARGSPPSVASSSASQLSAPSLKATCTANHDSPDAELIEANLLWRQEMGGNITRVESENKVVILDSFDPLVAEEDEREISVPAEILRKSRRFAQALPVWARPDYNPPLVETWKKPDYEPPVVHGCPLPPPKSPPVPPPRKKRTVVLTESTLSTALAELATRSFGSSSTSGITGDNTTTSSEPAPSGCPPDSDAESYSSMPPLEGEPGDPDLSDGSWSTVSSEANAEDVVCCSMSYSWTGALVTPCAAEEQKLPINALSNSLLRHHNLVYSTTSRSACQRQKKVTFDRLQVLDSHYQDVLKEVKAAASKVKANLLSVEEACSLTPPHSAKSKFGYGAKDVRCHARKAVTHINSVWKDLLEDNVTPIDTTIMAKNEVFCVQPEKGGRKPARLIVFPDLGVRVCEKMALYDVVTKLPLAVMGSSYGFQYSPGQRVEFLVQAWKSKKTPMGFSYDTRCFDSTVTESDIRTEEAIYQCCDLDPQARVAIKSLTERLYVGGPLTNSRGENCGYRRCRASGVLTTSCGNTLTCYIKARAACRAAGLQDCTMLVCGDDLVVICESAGVQEDAASLRAFTEAMTRYSAPPGDPPQPEYDLELITSCSSNVSVAHDGAGKRVYYLTRDPTTPLARAAWETARHTPVNSWLGNIIMFAPTLWARMILMTHFFSVLIARDQLEQALDCEIYGACYSIEPLDLPPIIQRLHGLSAFSLHSYSPGEINRVAACLRKLGVPPLRAWRHRARSVRARLLARGGRAAICGKYLFNWAVRTKLKLTPIAAAGQLDLSGWFTAGYSGGDIYHSVSHARPRWIWFCLLLLAAGVGIYLLPNR

SEQ ID NO: 2 - protein, 186 aa, Hepacivirus C
APITAYAQQTRGLLGCIITSLTGRDKNQVEGEVQIVSTATQTFLATCINGVCWAVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTD

SEQ ID NO: 3 - protein, 44 aa, Artificial Sequence
  Description of Artificial Sequence Synthetic polypeptide
TWVLVGGVLAALAAYCLSTGCVVIVGRIVLSGKPAIIPDREVLY

SEQ ID NO: 4 - protein, 20 aa, Artificial Sequence
  Description of Artificial Sequence Synthetic peptide
CMSADLEVVTSTWVLVGGVL

SEQ ID NO: 5 - protein, 20 aa, Artificial Sequence
  Description of Artificial Sequence Synthetic peptide
YQEFDEMEECSQHLPYIEQG

SEQ ID NO: 6 - protein, 20 aa, Artificial Sequence
  Description of Artificial Sequence Synthetic peptide
WISSECTTPCSGSWLRDIWD

SEQ ID NO: 7 - protein, 20 aa, Artificial Sequence
  Description of Artificial Sequence Synthetic peptide
GADTEDVVCCSMSYSWTGAL

SEQ ID NO: 8 - protein, 11 aa, Artificial Sequence
  Description of Artificial Sequence Synthetic peptide
ADLEVVTSTWL

SEQ ID NO: 9 - protein, 10 aa, Artificial Sequence
  Description of Artificial Sequence Synthetic peptide
DEMEECSQHL

SEQ ID NO: 10 - protein, 11 aa, Artificial Sequence
  Description of Artificial Sequence Synthetic peptide
ECTTPCSGSWL

SEQ ID NO: 11 - protein, 9 aa, Artificial Sequence
  Description of Artificial Sequence Synthetic peptide
EDVVPCSMG

SEQ ID NO: 12 - protein, 186 aa, Hepacivirus C
APITAYAQQTRGLLGCIITSLTGRDKNQVEGEVQIVSTAAQTFLATCINGVCWTVYHGAGTRTIASSKGPVIQMYTNVDQDLVGWPAPQGARSLTPCTCGSSDLYLVTRHADVIPVRRRGDGRGSLLSPRPISYLKGSSGGPLLCPAGHAVGIFRAAVCTRGVAKAVDFIPVEGLETTMRSPVFSD

SEQ ID NO: 13 - protein, 99 aa, Human immunodeficiency virus 1
PQVTLWQRPLVTIKIGGQLKEALLDTGADDTVLEEMSLPGRWKPKMIGGIGGFIKVRQYDQILIEICGHKAIGTVLVGPTPVNIIGRNLLTQIGCTLNF

SEQ ID NO: 14 - protein, 8 aa, Artificial Sequence
  Description of Artificial Sequence Synthetic peptide
EPLFAERK

SEQ ID NO: 15 - protein, 4 aa, Artificial Sequence
  Description of Artificial Sequence Synthetic peptide
YVAD

SEQ ID NO: 16 - protein, 5 aa, Artificial Sequence
  Description of Artificial Sequence Synthetic peptide
VDVAD

SEQ ID NO: 17 - protein, 4 aa, Artificial Sequence
  Description of Artificial Sequence Synthetic peptide
DEVD

SEQ ID NO: 18 - protein, 4 aa, Artificial Sequence
  Description of Artificial Sequence Synthetic peptide
VEHD

SEQ ID NO: 19 - protein, 4 aa, Artificial Sequence
  Description of Artificial Sequence Synthetic peptide
LGHD

SEQ ID NO: 20 - protein, 5 aa, Artificial Sequence
  Description of Artificial Sequence Synthetic peptide
LQTDG

SEQ ID NO: 21 - protein, 1306 aa, Homo sapiens
MGAASGRRGPGLLLPLPLLLLLPPQPALALDPGLQPGNFSADEAGAQLFAQSYNSSAEQVLFQSVAASWAHDTNITAENARRQEEAALLSQEFAEAWGQKAKELYEPIWQNFTDPQLRRIIGAVRTLGSANLPLAKRQQYNALLSNMSRIYSTAKVCLPNKTATCWSLDPDLTNILASSRSYAMLLFAWEGWHNAAGIPLKPLYEDFTALSNEAYKQDGFTDTGAYWRSWYNSPTFEDDLEHLYQQLEPLYLNLHAFVRRALHRRYGDRYINLRGPIPAHLLGDMWAQSWENIYDMVVPFPDKPNLDVTSTMLQQGWNATHMFRVAEEFFTSLELSPMPPEFWEGSMLEKPADGREVVCHASAWDFYNRKDFRIKQCTRVTMDQLSTVHHEMGHIQYYLQYKDLPVSLRRGANPGFHEAIGDVLALSVSTPEHLHKIGLLDRVTNDTESDINYLLKMALEKIAFLPFGYLVDQWRWGVFSGRTPPSRYNFDWWYLRTKYQGICPPVTRNETHFDAGAKFHVPNVTPYIRYFVSFVLQFQFHEALCKEAGYEGPLHQCDIYRSTKAGAKLRKVLQAGSSRPWQEVLKDMVGLDALDAQPLLKYFQPVTQWLQEQNQQNGEVLGWPEYQWHPPLPDNYPEGIDLVTDEAEASKFVEEYDRTSQVVWNEYAEANWNYNTNITTETSKILLQKNMQIANHTLKYGTQARKFDVNQLQNTTIKRIIKKVQDLERAALPAQELEEYNKILLDMETTYSVATVCHPNGSCLQLEPDLTNVMATSRKYEDLLWAWEGWRDKAGRAILQFYPKYVELINQAARLNGYVDAGDSWRSMYETPSLEQDLERLFQELQPLYLNLHAYVRRALHRHYGAQHINLEGPIPAHLLGNMWAQTWSNIYDLVVPFPSAPSMDTTEAMLKQGWTPRRMFKEADDFFTSLGLLPVPPEFWNKSMLEKPTDGREVVCHASAWDFYNGKDFRIKQCTTVNLEDLVVAHHEMGHIQYFMQYKDLPVALREGANPGFHEAIGDVLALSVSTPKHLHSLNLLSSEGGSDEHDINFLMKMALDKIAFIPFSYLVDQWRWRVFDGSITKENYNQEWWSLRLKYQGLCPPVPRTQGDFDPGAKFHIPSSVPYIRYFVSFIIQFQFHEALCQAAGHTGPLHKCDIYQSKEAGQRLATAMKLGFSRPWPEAMQLITGQPNMSASAMLSYFKPLLDWLRTENELHGEKLGWPQYNWTPNSARSEGPLPDSGRVSFLGLDLDAQQARVGQWLLLFLGIALLVATLGLSQRLFSIRHRSLHRHSHGPQFGSEVELRHS

SEQ ID NO: 22 - protein, 8 aa, Artificial Sequence
  Description of Artificial Sequence Synthetic peptide
EVNLDAEF

SEQ ID NO: 23 - protein, 7 aa, Artificial Sequence
  Description of Artificial Sequence Synthetic peptide
PQGIAGQ

SEQ ID NO: 24 - protein, 7 aa, Tobacco etch virus
ENLYFQS

SEQ ID NO: 25 - protein, 5 aa, Artificial Sequence
  Description of Artificial Sequence Synthetic peptide
HPFHL

SEQ ID NO: 26 - protein, 619 aa, Dengue virus
SGVLWDTPSPPEVERAVLDDGIYRIMQRGLLGRSQVGVGVFQDGVFHTMWHVTRGAVLMYQGKRLEPSWASVKKDLISYGGGWRFQGSWNTGEEVQVIAVEPGKNPKNVQTAPGTFKTPEGEVGAIALDFKPGTSGSPIVNREGKIVGLYGNGVVTTSGTYVSAIAQAKASQEGPLPEIEDEVFRKRNLTIMDLHPGSGKTRRYLPAIVREAIRRNVRTLILAPTRVVASEMAEALKGMPIRYQTTAVKSEHTGKEIVDLMCHATFTMRLLSPVRVPNYNMIIMDEAHFTDPASIARRGYISTRVGMGEAAAIFMTATPPGSVEAFPQSNAVIQDEERDIPERSWNSGYEWITDFPGKTVWFVPSIKSGNDIANCLRKNGKRVIQLSRKTFDTEYQKTKNNDWDYVVTTDISEMGANFRADRVIDPRRCLKPVILKDGPERVILAGPMPVTVASAAQRRGRIGRNQNKEGDQYVYMGQPLNNDEDHAHWTEAKMLLDNINTPEGIIPALFEPEREKSAAIDGEYRLRGEARKTFVELMRRGDLPVWLSYKVASEGFQYSDRRWCFDGERNNQVLEENMDVEMWTKEGERKKLRPRWLDARTYSDPLALREFKEFAAGRR

SEQ ID NO: 27 - protein, 618 aa, Dengue virus
AGVLWDVPSPPPVGKAELEDGAYRIKQKGILGYSQIGAGVYKEGTFHTMWHVTRGAVLMHKGKRIEPSWADVKKDLISYGGGWKLEGEWKEGEEVQVLALEPGKNPRAVQTKPGLFKTNAGTIGAVSLDFSPGTSGSPIIDKKGKVVGLYGNGVVTRSGAYVSAIAQTEKSIEDNPEIEDDIFRKRKLTIMDLHPGAGKTKRYLPAIVREAIKRGLRTLILAPTRVVAAEMEEALRGLPIRYQTPAIRAEHTGREIVDLMCHATFTMRLLSPVRVPNYNLIIMDEAHFTDPASIAARGYISTRVEMGEAAGIFMTATPPGSRDPFPQSNAPIMDEEREIPERSWSSGHEWVTDFKGKTVWFVPSIKAGNDIAACLRKNGKKVIQLSRKTFDSEYVKTRTNDWDFVVTTDISEMGANFKAERVIDPRRCMKPVILTDGEERVILAGPMPVTHSSAAQRRGRIGRNPKNENDQYIYMGEPLENDEDCAHWKEAKMLLDNINTPEGIIPSMFEPEREKVDAIDGEYRLRGEARKTFVDLMRRGDLPVWLAYRVAAEGINYADRRWCFDGIKNNQILEENVEVEIWTKEGERKKLKPRWLDAKIYSDPLALKEFKEFAAGRK

SEQ ID NO: 28 - protein, 619 aa, Dengue virus
SGVLWDVPSPPETQKAELEEGVYRIKQQGIFGKTQVGVGVQKEGVFHTMWHVTRGAVLTHNGKRLEPNWASVKKDLISYGGGWRLSAQWQKGEEVQVIAVEPGKNPKNFQTMPGIFQTTTGEIGAIALDFKPGTSGSPIINREGKVVGLYGNGVVTKNGGYVSGIAQTNAEPDGPTPELEEEMFKKRNLTIMDLHPGSGKTRKYLPAIVREAIKRRLRTLILAPTRVVAAEMEEALKGLPIRYQTTATKSEHTGREIVDLMCHATFTMRLLSPVRVPNYNLIIMDEAHFTDPASIAARGYISTRVGMGEAAAIFMTATPPGTADAFPQSNAPIQDEERDIPERSWNSGNEWITDFVGKTVWFVPSIKAGNDIANCLRKNGKKVIQLSRKTFDTEYQKTKLNDWDFVVTTDISEMGANFKADRVIDPRRCLKPVILTDGPERVILAGPMPVTVASAAQRRGRVGRNPQKENDQYIFMGQPLNKDEDHAHWTEAKMLLDNINTPEGIIPALFEPEREKSAAIDGEYRLKGESRKTFVELMRRGDLPVWLAHKVASEGIKYTDRKWCFDGERNNQILEENMDVEIWTKEGEKKKLRPRWLDARTYSDPLALKEFKDFAAGRK

SEQ ID NO: 29 - protein, 618 aa, Dengue virus
SGALWDVPSPAATQKAALSEGVYRIMQRGLFGKTQVGVGIHIEGVFHTMWHVTRGSVICHETGRLEPSWADVRNDMISYGGGWRLGDKWDKEEDVQVLAIEPGKNPKHVQTKPGLFKTLTGEIGAVTLDFKPGTSGSPIINRKGKVIGLYGNGVVTKSGDYVSAITQAERIGEPDYEVDEDIFRKKRLTIMDLHPGAGKTKRILPSIVREALKRRLRTLILAPTRVVAAEMEEALRGLPIRYQTPAVKSEHTGREIVDLMCHATFTTRLLSSTRVPNYNLIVMDEAHFTDPSSVAARGYISTRVEMGEAAAIFMTATPPGTTDPFPQSNSPIEDIEREIPERSWNTGFDWITDYQGKTVWFVPSIKAGNDIANCLRKSGKKVIQLSRKTFDTEYPKTKLTDWDFVVTTDISEMGANFRAGRVIDPRRCLKPVILPDGPERVILAGPIPVTPASAAQRRGRIGRNPAQEDDQYVFSGDPLKNDEDHAHWTEAKMLLDNIYTPEGIIPTLFGPEREKTQAIDGEFRLRGEQRKTFVELMRRGDLPVWLSYKVASAGISYKDREWCFTGERNNQILEENMEVEIWTREGEKKKLRPKWLDARVYADPMALKDFKEFASGRK

SEQ ID NO: 30 - protein, 10 aa, Hepacivirus C
GLLGCIITSL

SEQ ID NO: 31 - protein, 26 aa, Hepacivirus C
GEVQIVSTAAQTFLATCINGVCWTVY

SEQ ID NO: 32 - protein, 15 aa, Hepacivirus C
GEVQIVSTAAQTFLA

SEQ ID NO: 33 - protein, 15 aa, Hepacivirus C
QTFLATCINGVCWTV

SEQ ID NO: 34 - protein, 10 aa, Hepacivirus C
CINGVCWTVY

SEQ ID NO: 35 - protein, 15 aa, Hepacivirus C
SSDLYLVTRHADVIP

SEQ ID NO: 36 - protein, 8 aa, Hepacivirus C
YLVTRHAD

SEQ ID NO: 37 - protein, 9 aa, Hepacivirus C
LLCPAGHAV

SEQ ID NO: 38 - protein, 15 aa, Hepacivirus C
AVDFIPVEGLETTMR

SEQ ID NO: 39 - protein, 15 aa, Hepacivirus C
KIDTKYIMTCMSADL

SEQ ID NO: 40 - protein, 42 aa, Artificial Sequence
  Description of Artificial Sequence Synthetic polypeptide
PITKIDTKYIMTCMSADLEVVTSTWVLVGGVLAALAAYCLST

SEQ ID NO: 41 - protein, 5 aa, Artificial Sequence
  Description of Artificial Sequence Synthetic peptide
KKKRK

SEQ ID NO: 42 - protein, 26 aa, Artificial Sequence
  Description of Artificial Sequence Synthetic peptide
MLRTSSLFTRRVQPSLFRNILRLQST

SEQ ID NO: 43 - protein, 4 aa, Artificial Sequence
  Description of Artificial Sequence Synthetic peptide
KDEL

SEQ ID NO: 44 - protein, 304 aa, Artificial Sequence
  Description of Artificial Sequence Synthetic polypeptide
DEMEECSQHLPGAGSSGDIMDYKDDDDKGSSGTGSGSGTSAPITAYAQQTRGLLGCIITSLTGRDKNQVEGEVQIVSTATQTFLATCINGVCWAVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAVTLTHPITKIDTKYIMTCMSADLEVVTSTWVLVGGVLAALAAYCLSTGCVVIVGRIVLSGKPAIIPDREVLY

SEQ ID NO: 45 - protein, 303 aa, Artificial Sequence
  Description of Artificial Sequence Synthetic polypeptide
MDYKDDDDKGSSGTGSGSGTSAPITAYAQQTRGLLGCIITSLTGRDKNQVEGEVQIVSTATQTFLATCINGVCWAVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAVTLTHPITKIDTKYIMTCMSADLEVVTSTWVLVGGVLAALAAYCLSTGCVVIVGRIVLSGKPAGSSGSSIIPDREVLYQEFEDVVPCSMG

SEQ ID NO: 46 - protein, 64 aa, Artificial Sequence
  Description of Artificial Sequence Synthetic polypeptide
LQMLPESEDEESYDTESEFTEFTEDELPYDDGSLQMLPESEDEESYDTESEFTEFTEDELPYDD

SEQ ID NO: 47 - protein, 57 aa, Homo sapiens
EIKDKEEVQRKRQKLMPNFSDSFGGGSGAGAGGGGMFGSGGGGGGTGSTGPGYSFPH

SEQ ID NO: 48 - protein, 86 aa, Unknown
  Description of Unknown yeast sequence
IDDENGSVILQDDDYDDGNNHIPFEDDDVYNYNDNDDDDERIEFEDDDDDDDDSIDNDSVMDRKQPHKAEDESEDVEDVERVSKKD

SEQ ID NO: 49 - protein, 41 aa, Artificial Sequence
  Description of Artificial Sequence Synthetic polypeptide
PESMREEYRKEGSKRIKCPDCEPFCNKRGSPESMREEYRKE

SEQ ID NO: 50 - protein, 68 aa, Artificial Sequence
  Description of Artificial Sequence Synthetic polypeptide
RSYSPTSPNYSPTSPSGSYSPTSPNYSPTSPSGGSRSYSPTSPNYSPTSPSGSYSPTSPNYSPTSPSG

SEQ ID NO: 51 - protein, 59 aa, Artificial Sequence
  Description of Artificial Sequence Synthetic polypeptide
PESMREEYRKEGSSLLTEVETPGSPESMREEYRKEGSSLLTEVETPGSPESMREEYRKE

SEQ ID NO: 52 - protein, 51 aa, Artificial Sequence
  Description of Artificial Sequence Synthetic polypeptide
LIEEVRHRLKTTENSGSLIEEVRHRLKTTENSGSLIEEVRHRLKTTENSGS

SEQ ID NO: 53 - protein, 37 aa, Unknown
  Description of Unknown ornithine decarboxylase sequence
FPPEVEEQDDGTLPMSCAQESGMDRHPAACASARINV

SEQ ID NO: 54 - protein, 40 aa, Unknown
  Description of Unknown mODC DA sequence
SHGFPPEVEEQAAGTLPMSCAQESGMDRHPAACASARINV

SEQ ID NO: 55 - protein, 6 aa, Artificial Sequence
  Description of Artificial Sequence Synthetic peptide
  MOD_RES (4)..(5): Any amino acid
DSGXXS

SEQ ID NO: 56 - protein, 7 aa, Artificial Sequence
  Description of Artificial Sequence Synthetic peptide
  MOD_RES (1)..(1): Abz
  MOD_RES (7)..(7): Lys(Dnp)
XHPFHLK

(19) United States
(12) Patent Application Publication
(10) Pub. No.: US 2022/0090040 A1
(43) Pub. Date: Mar. 24, 2022

(54) Fusion Constructs for Controlling Protein Function

(71) Applicant: Senti Biosciences, Inc., South San Francisco, CA (US)

(72) Inventors: Daniel Frimannsson, Alameda, CA (US); Philip Janmin Lee, Alameda, CA (US); Timothy Kuan-Ta Lu, San Francisco, CA (US); Russell Morrison Gordley, San Francisco, CA (US)

(73) Assignees: Senti Biosciences, Inc., South San Francisco, CA (US); Senti Biosciences, Inc., South San Francisco, CA (US)

(21) Appl. No.: 17/425,609

(22) PCT Filed: Jan. 24, 2020
(86) PCT No.: PCT/US20/15011

§ 371 Date: Jul. 23, 2021

Related U.S. Application Data
(60) Provisional application No. 62/797,043, filed on Jan. 25, 2019.

(51) Int. Cl. C12N 9/50; C07K 14/725; C07K 14/005; A61K 35/17

(52) U.S. Cl. CPC C12N 9/506; C07K 14/7051; C07K 14/005; C07K 2319/03; C07K 2319/50; C12N 2770/24222; A61K 35/17

(57) ABSTRACT

Described herein are engineered fusion proteins comprising a variant protease (e.g., an HCV NS3 protease) fused to a polypeptide of interest and a cognate protease cleavage site. The cleavability of the cognate protease cleavage site enables the controllability of one or more functions of the polypeptide of interest. Additionally disclosed are methods for generating engineered fusion proteins as well as their therapeutic use.

### CROSS REFERENCE TO RELATED APPLICATIONS

This application claims the benefit of and priority to U.S. Provisional Application No. 62/797,043, filed Jan. 25, 2019, which is hereby incorporated by reference in its entirety for all purposes.

### SEQUENCE LISTING

The instant application contains a Sequence Listing which has been submitted electronically in ASCII format and is hereby incorporated by reference in its entirety. Said ASCII copy, created on Jan. 17, 2020, is named STB-015WO_SL.txt and is 83,131 bytes in size.

### TECHNICAL FIELD

The present disclosure pertains generally to the field of protein engineering and methods of controlling the function of proteins. In particular, the present disclosure relates to engineered fusion proteins comprising a variant protease (e.g., an HCV NS3 protease) fused to a polypeptide of interest and a cognate protease cleavage site whose cleavage can be inhibited with a protease inhibitor such that one or more functions of the polypeptide of interest are controllable.

### BACKGROUND

Technology for rapidly shutting off the production and/or function of specific proteins in eukaryotes would be of widespread utility as a research tool and for gene or cell therapy applications, but a simple and effective method has yet to be developed.

Controlling protein production through repression of transcription is slow in onset, as existing mRNA molecules continue to be translated into proteins after transcriptional inhibition. RNA interference (RNAi) directly induces mRNA destruction, but RNAi is often only partially effective and can exhibit both sequence-independent and sequence-dependent off-target effects (Sigoillot et al. (2011) ACS Chem Biol 6:47-60). Furthermore, mRNA and protein abundance are not always correlated due to regulation of the translation rate of specific mRNAs (Vogel et al. (2012) Nat Rev Genet 13:227-232; Wu et al. (2013) Nature 499:79-82; Battle et al. (2015) Science 347:664-667). Lastly, both transcriptional repression and RNAi take days to reverse (Liu et al. (2008) J Gene Med 10:583-592; Matsukura et al. (2003) Nucleic Acids Res 31:e77).

Thus, there remains a need for a simple to use system for controlling protein production and function.

### BRIEF SUMMARY

In order to meet the above needs, the present disclosure relates to fusion constructs and methods of using them for controlling protein function and/or production. In particular, the present disclosure provides fusion proteins containing a variant protease (e.g., an HCV NS3 protease) fused to a polypeptide of interest and a cognate protease cleavage site whose cleavage can be inhibited with a protease inhibitor such that one or more functions of the polypeptide of interest are controllable.

Accordingly, certain aspects of the present disclosure provide a fusion protein, having a polypeptide of interest; a variant hepatitis C virus (HCV) nonstructural protein 3 (NS3) protease; and a cognate protease cleavage site, where the variant HCV NS3 protease comprises one or more mutations; and where the one or more mutations decrease immunogenicity when the fusion protein is expressed in a mammalian cell. In some embodiments, the HCV NS3 protease is derived from an HCV polyprotein comprising an amino acid sequence having at least about 80-100% sequence identity to SEQ ID NO: 1, including any percent identity within this range, such as 81, 82, 83, 84, 85, 86, 87, 88, 89, 90, 91, 92, 93, 94, 95, 96, 97, 98, or 99% sequence identity to SEQ ID NO: 1. In some embodiments, the variant NS3 protease is derived from an HCV NS3 protease having the amino acid sequence of APITAYAQQT RGLLGCIITS LTGRDKNQVE GEVQIVSTAT QTFLATCING VCWAVYHGAG TRTIASPKGP VIQMYTNVDQ DLVGWPAPQG SRSLTPCTCG SSDLYLVTRH ADVIPVRRRG DSRGSLLSPR PISYLKGSSG GPLLCPAGHA VGLFRAAVCT RGVAKAVDFI PVENLETTMR SPVFTD (SEQ ID NO: 2).

In some embodiments that may be combined with any of the preceding embodiments, the one or more mutations comprise one or more amino acid substitutions. In some embodiments that may be combined with any of the preceding embodiments, the one or more amino acid substitutions correspond to amino acid substitutions within SEQ ID NO: 1. In some embodiments that may be combined with any of the preceding embodiments, the one or more amino acid substitutions are at one or more positions corresponding to positions 1038 to 1047 of SEQ ID NO: 1, positions 1057 to 1081 of SEQ ID NO: 1, positions 1073 to 1081 of SEQ ID NO: 1, positions 1073 to 1082 of SEQ ID NO: 1, positions 1127 to 1141 of SEQ ID NO 1, positions 1131 to 1138 of SEQ ID NO 1, positions 1169 to 1177 of SEQ ID NO. 1, and/or positions 1192 to 1206 of SEQ ID NO: 1. In some embodiments that may be combined with any of the preceding embodiments, the one or more amino acid substitutions are selected from a position corresponding to position 1062 of SEQ ID NO: 1, a position corresponding to position 1069 of SEQ ID NO. 1, a position corresponding to position 1070 of SEQ ID NO 1, a position corresponding to position 1071 of SEQ ID NO: 1, a position corresponding to position 1072 of SEQ ID NO 1, a position corresponding to position 1074 of SEQ ID NO. 1, a position corresponding to position 1075 of SEQ ID NO: 1, a position corresponding to position 1077 of SEQ ID NO: 1, a position corresponding to position 1078 of SEQ ID NO: 1, a position corresponding to position 1079 of SEQ ID NO: 1, a position corresponding to position 1080 of SEQ ID NO: 1, a position corresponding to position 1031 of SEQ ID NO: 1, a position corresponding to position 1074 of SEQ ID NO: 1, a position corresponding to position 1132 of SEQ ID NO: 1, a position corresponding to position 1133 of SEQ ID NO: 1, a position corresponding to position 1195 of SEQ ID NO: 1, a position corresponding to position 1196 of SEQ ID NO: 1, a position corresponding to position 1201 of SEQ ID NO: 1, a position corresponding to position 1202 of SEQ ID NO: 1, and any combination thereof. In some embodiments that may be combined with any of the preceding embodiments, the one or more amino acid substitutions are selected from an Ile to Leu substitution at a position corresponding to position 1074 of SEQ ID NO. 1, an Ile to Met substitution at a position corresponding to position 1074 of SEQ ID NO: 1, an Asn to Ala substitution at a position corresponding to position 1075 of SEQ ID NO. 1, a Val to Ala substitution at a position corresponding to position 1077 of SEQ ID NO: 1, a Cys to Phe substitution at a position corresponding to position 1078 of SEQ ID NO. 1, a Trp to Ala substitution at a position corresponding to position 1079 of SEQ ID NO: 1, a Thr to Ala substitution at a position corresponding to position 1080 of SEQ ID NO: 1, a Val to Ala substitution at a position corresponding to position 1081 of SEQ ID NO: 1, a Val to Asn substitution at a position corresponding to position 1081 of SEQ ID NO: 1, and any combination thereof. In some embodiments that may be combined with any of the preceding embodiments, the one or more amino acid substitutions comprise a Thr to Ala substitution at a position corresponding to position 1080 of SEQ ID NO: 1. In some embodiments that may be combined with any of the preceding embodiments, the one or more amino acid substitutions comprise a Thr to Ala substitution at a position corresponding to position 1080 of SEQ ID NO: 1 and a Val to Ala substitution at a position corresponding to position 1077 of SEQ ID NO: 1. In some embodiments that may be combined with any of the preceding embodiments, the one or more amino acid substitutions comprise a Thr to Ala substitution at a position corresponding to position 1080 of SEQ ID NO: 1 and a Val to Ala substitution at a position corresponding to position 1081 of SEQ ID NO 1.

In some embodiments that may be combined with any of the preceding embodiments, the fusion protein further comprises an HCV NS4A co-factor. In some embodiments, the NS4A co-factor has the amino acid sequence of TWVLVGGVLA ALAAYCLSTG CVVIVGRIVL SGKPAIIPDR EVLY (SEQ ID NO: 3).

In some embodiments that may be combined with any of the preceding embodiments, wherein the fusion protein further comprises a degron, wherein the degron is operably linked to the polypeptide of interest. In some embodiments that may be combined with any of the preceding embodiments, the degron is selected from HCV NS4 degron, PEST (two copies of residues 277-307 of human IκBα) (SEQ ID NO: 46), GRR (residues 352-408 of human p105) (SEQ ID NO: 47), DRR (residues 210-295 of yeast Cdc34) (SEQ ID NO: 48), SNS (tandem repeat of SP2 and NB (SP2-NB-SP2 of influenza A or influenza B) (SEQ ID NO: 49), RPB (four copies of residues 1688-1702 of yeast RPB) (SEQ ID NO: 50), SPmix (tandem repeat of SP1 and SP2 (SP2-SP1-SP2-SP1-SP2 of influenza A virus M2 protein) (SEQ ID NO: 51), NS2 (three copies of residues 79-93 of influenza A virus NS protein) (SEQ ID NO: 52), ODC (residues 106-142 of ornithine decarboxylase) (SEQ ID NO: 53), Nek2A, mouse ODC (residues 422-461), mouse ODC_DA (residues 422-461 of mODC including D433A and D434A point mutations) (SEQ ID NO: 54), an APC/C degron, a COP1 E3 ligase binding degron motif, a CRL4-Cdt2 binding PIP degron, an actinfilin-binding degron, a KEAP1 binding degron, a KLHL2 and KLHL3 binding degron, an MDM2 binding motif, an N-degron, a hydroxyproline modification in hypoxia signaling, a phytohormone-dependent SCF-LRR-binding degron, an SCF ubiquitin ligase binding phosphodegron, a phytohormone-dependent SCF-LRR-binding degron, a DSGxxS (SEQ ID NO: 55) phospho-dependent degron, an Siah binding motif, an SPOP SBC docking motif, and a PCNA binding PIP box.

In some embodiments that may be combined with any of the preceding embodiments, the variant HCV NS3 protease comprises one or more additional mutations. In some embodiments that may be combined with any of the preceding embodiments, the one or more additional mutations modulate enzymatic activity of the variant HCV NS3 protease. In some embodiments that may be combined with any of the preceding embodiments, the one or more additional mutations are one or more additional amino acid substitutions. In some embodiments that may be combined with any of the preceding embodiments, the one or more additional amino acid substitutions are at one more positions corresponding to position 1074 of SEQ ID NO: 1, position 1078 of SEQ ID NO: 1 and/or position 1079 of SEQ ID NO: 1. In some embodiments that may be combined with any of the preceding embodiments, the one or more additional amino acid substitutions are selected from an Ile to Ala substitution at a position corresponding to position 1074 of SEQ ID NO: 1, a Trp to Ala substitution at a position corresponding to position 1079 of SEQ ID NO. 1, and any combination thereof. In some embodiments that may be combined with any of the preceding embodiments, the one or more additional amino acid substitutions decrease enzymatic activity of the variant HCV NS3 protease. In some embodiments that may be combined with any of the preceding embodiments, the one or more additional amino acid substitutions comprise a Cys to Ala substitution at a position corresponding to position 1078 of SEQ ID NO: 1. In some embodiments that may be combined with any of the preceding embodiments, the one or more additional amino acid substitutions increase enzymatic activity of the variant HCV NS3 protease.

In some embodiments that may be combined with any of the preceding embodiments, the cognate protease cleavage site comprises an amino acid sequence selected from any of the amino acid sequences listed in Table 1. In some embodiments that may be combined with any of the preceding embodiments, the cognate protease cleavage site comprises an amino acid sequence selected from CMSADLEVVTSTWVLVGGVL (SEQ ID NO: 4), YQEFDEMEECSQHLPYIEQG (SEQ ID NO. 5), WISSECTTPCSGSWLRDIWD (SEQ ID NO: 6), and GADTEDVVCCSMSYSWTGAL (SEQ ID NO: 7). In some embodiments that may be combined with any of the preceding embodiments, the cognate protease cleavage site comprises an amino acid sequence selected from ADLEVVTSTWL (SEQ ID NO: 8), DEMEECSQHL (SEQ ID NO: 9), ECTTPCSGSWL (SEQ ID NO: 10), and EDVVPCSMG (SEQ ID NO: 11). In some embodiments that may be combined with any of the preceding embodiments, the cognate protease cleavage site comprises one or more mutations. In some embodiments that may be combined with any of the preceding embodiments, the one or more mutations comprise one or more amino acid substitutions. In some embodiments that may be combined with any of the preceding embodiments, the one or more mutations increase the catalytic rate of cleavage. In some embodiments that may be combined with any of the preceding embodiments, the one or more mutations decrease the catalytic rate of cleavage.

In some embodiments that may be combined with any of the preceding embodiments, the polypeptide of interest is selected from a membrane protein, a receptor, a hormone, a cytokine, a transport protein, a transcription factor, a cytoskeletal protein, an extracellular matrix protein, a signal-transduction protein, and an enzyme. In some embodiments that may be combined with any of the preceding embodiments, the polypeptide of interest comprises a biologically active domain of a protein. In some embodiments that may be combined with any of the preceding embodiments, the biologically active domain is a catalytic domain, a ligand binding domain, or a protein-protein interaction domain. In some embodiments that may be combined with any of the preceding embodiments, the polypeptide of interest is a receptor selected from a T cell receptor (TCR), a chimeric T cell receptor, an artificial T cell receptor, a synthetic T cell receptor, a chimeric immunoreceptor, an antibody-coupled T cell receptor (ACTR), a T cell receptor fusion construct (TRUC), and a chimeric antigen receptor (CAR). In some embodiments that may be combined with any of the preceding embodiments, the polypeptide of interest is a chimeric antigen receptor (CAR). In some embodiments that may be combined with any of the preceding embodiments, the polypeptide of interest is a cytokine. In some embodiments that may be combined with any of the preceding embodiments, the cytokine is a proinflammatory cytokine. In some embodiments that may be combined with any of the preceding embodiments, the cognate protease cleavage site is localized within a domain of the polypeptide of interest. In some embodiments that may be combined with any of the preceding embodiments, the polypeptide of interest comprises multiple domains. In some embodiments that may be combined with any of the preceding embodiments, the cognate protease cleavage site is localized between the multiple domains of the polypeptide of interest.

In some embodiments that may be combined with any of the preceding embodiments, the variant HCV NS3 protease can be repressed by a protease inhibitor. In some embodiments that may be combined with any of the preceding embodiments, the protease inhibitor is selected from simeprevir, danoprevir, asunaprevir, ciluprevir, boceprevir, sovaprevir, paritaprevir, telaprevir, grazoprevir, glecaprevir, and voxiloprevir. In some embodiments that may be combined with any of the preceding embodiments, wherein the fusion protein further comprises a targeting sequence. In some embodiments that may be combined with any of the preceding embodiments, the targeting sequence is selected from a secretory protein signal sequence, a membrane protein signal sequence, a nuclear localization sequence, a nucleolar localization signal sequence, an endoplasmic reticulum localization sequence, a peroxisome localization sequence, a mitochondrial localization sequence, and a protein binding motif sequence.

Other aspects of the present disclosure relate to a polynucleotide encoding the fusion protein of any of the preceding embodiments. Other aspects of the present disclosure relate to a vector comprising the polynucleotide of any of the preceding embodiments. Other aspects of the present disclosure relate to a cell comprising a fusion protein of any of the preceding embodiments, a polynucleotide of any of the preceding embodiments, or a vector of any of the preceding embodiments. In some embodiments that may be combined with any of the preceding embodiments, wherein the cell is an immune cell or a cell line derived from an immune cell. In some embodiments that may be combined with any of the preceding embodiments, the immune cell is selected from a T cell, a B cell, an NK cell, an NKT cell, an innate lymphoid cell, a mast cell, an eosinophil, a basophils, a macrophage, a neutrophil, a dendritic cell, and any combinations thereof. In some embodiments that may be combined with any of the preceding embodiments, the cell is a mesenchymal stromal cell. Other aspects of the present disclosure relate to a pharmaceutical composition comprising the fusion protein of any of the preceding embodiments and an excipient. Other aspects of the present disclosure relate to a pharmaceutical composition comprising the cell of any of the preceding embodiments and an excipient.

Other aspects of the present disclosure relate to a method of treating a subject in need thereof, comprising administering the pharmaceutical composition of any of the preceding embodiments.

Other aspects of the present disclosure relate to a method of regulating activity of a protein of interest, comprising: a) providing a population of cells comprising the fusion protein of any of the preceding embodiments, the polynucleotide of any of the preceding embodiments, or the vector of any of the preceding embodiments; and b) contacting the population of cells with a protease inhibitor. In some embodiments that may be combined with any of the preceding embodiments, the method further comprises the step of removing the protease inhibitor from the population of cells in some embodiments that may be combined with any of the preceding embodiments, the method further comprises the step of administering the population of cells to a subject in need of a cell-based therapy.

Other aspects of the present disclosure relate to a method of treating a subject in need of a cell-based therapy, comprising administering to the subject a population of cells comprising the fusion protein of any of the preceding embodiments, the polynucleotide of any of the preceding embodiments, or the vector of any of the preceding embodiments. In some embodiments that may be combined with any of the preceding embodiments, the population of cells was cultured in the presence of a protease inhibitor capable of inhibiting the repressible protease. In some embodiments that may be combined with any of the preceding embodiments, the population of cells was cultured in the absence of a protease inhibitor capable of inhibiting the repressible protease. In some embodiments that may be combined with any of the preceding embodiments, the method further comprises the step of administering to the subject the protease inhibitor capable of inhibiting the repressible protease. In some embodiments that may be combined with any of the preceding embodiments, the method further comprises the step of withdrawing the protease inhibitor capable of inhibiting the repressible protease from the subject.

In another aspect, the present disclosure includes a fusion protein comprising: a) a polypeptide of interest; b) a degron, wherein the degron is operably linked to the polypeptide of interest when the fusion protein is in an uncleaved state, such that the degron promotes degradation of the polypeptide of interest in a cell, c) a variant protease, wherein the variant protease can be inhibited by contacting the fusion protein with a protease inhibitor; and c) a cleavable linker that is located between the polypeptide of interest and the degron, wherein the cleavable linker comprises a cognate cleavage site recognized by the protease, wherein cleavage of the cleavable linker by the protease releases the polypeptide of interest from the fusion protein, such that when the fusion protein is in a cleaved state, the degron no longer controls degradation of the polypeptide of interest.

In some embodiments, the degron may be linked to the C-terminus of the polypeptide of interest in the fusion protein. In certain embodiments, the fusion protein comprises components arranged from N-terminus to C-terminus in the uncleaved state as follows: a) the polypeptide of interest, b) the cleavable linker, c) the variant protease, and d) the degron.

Alternatively, the degron may be linked to the N-terminus of the polypeptide of interest in the fusion protein. In certain embodiments, the fusion protein comprises components arranged from N-terminus to C-terminus in the uncleaved state as follows a) the variant protease, b) the degron, c) the cleavable linker, and c) the polypeptide of interest. Exemplary targeting sequences include a secretory protein signal sequence, a membrane protein signal sequence, a nuclear localization sequence, a nucleolar localization signal sequence, an endoplasmic reticulum localization sequence, a peroxisome localization sequence, a mitochondrial localization sequence, and a protein binding motif sequence.

In certain embodiments, the fusion protein further comprises a tag Exemplary tags include a His-tag, a Strep-tag, a TAP-tag, an S-tag, an SBP-tag, an Arg-tag, a calmodulin-binding peptide tag, a cellulose-binding domain tag, a DsbA tag, a c-myc tag, a glutathione S-transferase tag, a FLAG tag, a HAT-tag, a maltose-binding protein tag, a NusA tag, and a thioredoxin tag.

In certain embodiments, the fusion protein further comprises a detectable label. The detectable label may comprise any molecule capable of detection. For example, the detectable label may be a fluorescent, bioluminescent, chemiluminescent, colorimetric, or isotopic label. In certain embodiments, the detectable label is a fluorescent protein or bioluminescent protein.

In certain embodiments, the polypeptide of interest in fusion protein is a membrane protein, a receptor, a hormone, a transport protein, a transcription factor, a cytoskeletal protein, an extracellular matrix protein, a signal-transduction protein, an enzyme, or any other protein of interest. The polypeptide of interest may comprise an entire protein, or a biologically active domain (e.g., a catalytic domain, a ligand binding domain, or a protein-protein interaction domain), or a polypeptide fragment of a selected protein of interest.

In another aspect, the present disclosure includes a polynucleotide encoding a fusion protein described herein. In one embodiment, the polynucleotide is a recombinant polynucleotide comprising a polynucleotide encoding a fusion protein operably linked to a promoter. The recombinant polynucleotide may comprise an expression vector, for example, a bacterial plasmid vector or a viral expression vector. Exemplary viral vectors include measles virus, vesicular stomatitis virus, adenovirus, retrovirus (e.g., γ-retrovirus and lentivirus), poxvirus, adeno-associated virus, baculovirus, or herpes simplex virus vectors.

In another aspect, the present disclosure includes a host cell comprising a recombinant polynucleotide encoding a fusion protein operably linked to a promoter. In one embodiment, the host cell is a eukaryotic cell. In another embodiment, the host cell is a mammalian cell. In certain embodiments, the host cell is a stem cell (e.g., embryonic stem cell or adult stein cell). Host cells may be cultured as unicellular or multicellular entities (e.g., tissue, organs, or organoids comprising the recombinant vector). The promoter may be an endogenous or exogenous promoter. In certain embodiments, the recombinant polynucleotide encoding the fusion protein resides on an extrachromosomal plasmid or vector in other embodiments, the recombinant polynucleotide encoding the fusion protein is integrated into the cellular genome. For example, the recombinant polynucleotide may integrate into the cellular genome at a position where the polynucleotide sequence encoding the fusion protein is operably linked to an endogenous promoter of a gene. In another embodiment, the present disclosure includes a descendant of the host cell, wherein the descendant has inherited a recombinant polynucleotide encoding the fusion protein.

In another embodiment, the present disclosure includes an organoid comprising a recombinant polynucleotide encoding a fusion protein operably linked to a promoter. The promoter may be an endogenous or exogenous promoter. In certain embodiments, the recombinant polynucleotide encoding the fusion protein resides on an extrachromosomal plasmid or vector. In other embodiments, the recombinant polynucleotide encoding the fusion protein is integrated into the organoid genome. For example, the recombinant polynucleotide may integrate into the organoid genome at a position where the polynucleotide sequence encoding the fusion protein is operably linked to an endogenous promoter of a gene. In another embodiment, the present disclosure includes a recombinant animal comprising a recombinant polynucleotide encoding a fusion protein operably linked to a promoter. The promoter may be an endogenous or exogenous promoter. In certain embodiments, the recombinant polynucleotide encoding the fusion protein resides on an extrachromosomal plasmid or vector. In other embodiments, the recombinant polynucleotide encoding the fusion protein is integrated into the genome of the recombinant animal. For example, the recombinant polynucleotide may integrate into the genome at a position where the polynucleotide sequence encoding the fusion protein is operably linked to an endogenous promoter of a gene. In another embodiment, the present disclosure includes a descendant of the recombinant animal, wherein the descendant has inherited the recombinant polynucleotide encoding the fusion protein.

In another aspect, the present disclosure includes a method for producing a fusion protein, the method comprising: transforming a host cell with a recombinant polynucleotide encoding the fusion protein operably linked to a promoter, culturing the transformed host cell under conditions whereby the fusion protein is expressed; and isolating the fusion protein from the host cell.

In another aspect, the present disclosure includes a method for controlling production of a polypeptide of interest, the method comprising: a) transforming a host cell with a recombinant polynucleotide encoding fusion protein described herein; b) culturing the transformed host cell under conditions whereby the fusion protein is expressed; and c) contacting the cell with a protease inhibitor that inhibits the protease of the fusion protein when production of the polypeptide of interest is no longer desired. The protease inhibitor can be removed when resuming production of the polypeptide of interest is desired.

The recombinant polynucleotide encoding the fusion protein preferably is capable of providing efficient production of the polypeptide of interest with biological activity comparable to the wild-type polypeptide. Additionally, production of the polypeptide of interest from the recombinant polynucleotide preferably can be rapidly and nearly completely suppressed in the presence of a protease inhibitor. For example, a protease inhibitor may reduce production of the polypeptide of interest by at least 80%, 90%, or 100%, or any amount in between as compared to levels of the polypeptide in the absence of the protease inhibitor. In certain embodiments, production of the polypeptide of interest by the recombinant polynucleotide in the host cell in the presence of the protease inhibitor is at least about 90% to 100% suppressed, including any percent identity within this range, such as 90, 91, 92, 93, 94, 95, 96, 97, 98, or 99%.

In certain embodiments, the fusion protein used for controlling production of a polypeptide of interest comprises an HCV NS3 protease. NS3 protease inhibitors that can be used in the practice of the present disclosure include, but are not limited to, simeprevir, danoprevir, asunaprevir, ciluprevir, boceprevir, sovaprevir, paritaprevir and telaprevir.

In another aspect, the present disclosure includes a method for controlling production of a polypeptide of interest in a subject, the method comprising a) administering a recombinant polynucleotide encoding a fusion protein to the subject, such that the fusion protein is expressed in the subject; and b) administering a protease inhibitor that inhibits the protease of the fusion protein to the subject when production of the polypeptide of interest is not desired. The method may further comprise ceasing administration of the protease inhibitor when resuming production of the polypeptide of interest in the subject is desired. The recombinant polynucleotide may comprise an expression vector, for example, a viral expression vector, such as, but not limited to, an adenovirus, retrovirus (e.g., y-retrovirus and lentivirus), poxvirus, adeno-associated virus, baculovirus, or herpes simplex virus vector. In one embodiment, the recombinant polynucleotide comprises a polynucleotide sequence encoding the fusion protein operably linked to an exogenous promoter. In another embodiment, the recombinant polynucleotide is integrated into the genome of the subject. For example, the recombinant polynucleotide may integrate into the genome at a position where the polynucleotide sequence encoding the fusion protein is operably linked to an endogenous promoter of a gene in the subject.

In another aspect, the present disclosure includes a method for controlling production of a polypeptide of interest in a recombinant animal, the method comprising: a) administering a recombinant polynucleotide encoding a fusion protein to the recombinant animal, such that the fusion protein is expressed in the recombinant animal and b) administering a protease inhibitor that inhibits the protease of the fusion protein to the recombinant animal when production of the polypeptide of interest is not desired. In another aspect, the present disclosure includes a method of controlling production of a polypeptide of interest in an organoid, the method comprising: a) introducing a recombinant polynucleotide encoding the fusion protein of claim 4 into an organoid; b) culturing the organoid under conditions whereby the fusion protein is produced in the organoid; and c) contacting the organoid with a protease inhibitor that inhibits the protease of the fusion protein when production of the polypeptide of interest is no longer desired.

In another aspect, the present disclosure includes a method of measuring the turnover of a polypeptide of interest, the method comprising: a) introducing a recombinant polynucleotide encoding a fusion protein described herein into a cell; b) measuring amounts of the polypeptide of interest in the cell before and after contacting the cell with a protease inhibitor that inhibits the protease of the fusion protein; and c) calculating the turnover of the polypeptide of interest based on the amounts of the polypeptide of interest in the cell before and after adding the protease inhibitor Additionally, the half-life of the polypeptide of interest in the cell can be calculated. The amount of the polypeptide of interest in the cell can be measured either continuously or periodically over a period of time.

In another aspect, the present disclosure includes a conditionally replicating viral vector comprising a modified genome of a virus such that production of a polypeptide required for efficient replication of the virus is controllable, wherein the viral vector comprises a nucleic acid encoding a fusion protein comprising: i) the polypeptide required for efficient replication of the virus; ii) a degron, wherein the degron is operably linked to the polypeptide required for efficient replication of the virus when the fusion protein is in an uncleaved state, such that the degron promotes degradation of the polypeptide in a cell; iii) a protease, wherein the protease can be inhibited by contacting said fusion protein with a protease inhibitor; and iv) a cleavable linker that is located between the polypeptide required for efficient replication of the virus and the degron, wherein the cleavable linker comprises a cleavage site recognized by the protease, wherein cleavage of the cleavable linker by the protease releases the polypeptide required for efficient replication of the virus from the fusion protein, such that when the fusion protein is in a cleaved state, the degron no longer controls degradation of the polypeptide required for efficient replication of the virus. In certain embodiments, the virus is an RNA virus (e.g., measles virus or a vesicular stomatitis vims) in another embodiment, the conditionally replicating viral vector is a plasmid. The viral vector may further comprise a multiple cloning site, transcription promoter, transcription enhancer element, transcription termination signal, polyadenylation sequence, or exogenous nucleic acid, or any combination thereof.

In another aspect, the present disclosure includes a method of controlling production of a virus, the method comprising: a) introducing a conditionally replicating viral vector described herein into a host cell; b) culturing the host cell under conditions suitable for producing the virus; and c) contacting the host cell with a protease inhibitor, such that the polypeptide required for efficient replication of the virus is degraded when production of the virus is no longer desired. The protease inhibitor can be removed when resuming production of the virus is desired.

The conditionally replicating viral vector preferably is capable of providing efficient production of the virus in the host cell in the absence of a protease inhibitor, comparable to the level of the virus produced by the wild-type viral genome. In certain embodiments, the level of the virus produced by the conditionally replicating viral vector in the absence of the protease inhibitor is at least about 30%, 35%, 40%, 45%, 50%, 55%, 60%, 65%, 70%, 75%, 80%, 85%, 90%, 95%, or 100%, or any amount in between as compared to levels of the virus produced by the wild-type viral genome.

Additionally, production of the virus from the conditionally replicating viral vector preferably can be nearly completely suppressed in the presence of a protease inhibitor. For example, a protease inhibitor may reduce production of the virus by 80%, 90%, 100%, or any amount in between as compared to levels of the virus in the absence of the protease inhibitor. In certain embodiments, production of the virus by the conditionally replicating viral vector in the host cell in the presence of the protease inhibitor is at least about 90% to 100% suppressed, including any percent identity within this range, such as 90, 91, 92, 93, 94, 95, 96, 97, 98, or 99%.

In certain embodiments, the conditionally replicating viral vector, used in controlling production of a virus, expresses a fusion protein comprising an HCV NS3 protease, wherein addition of an NS3 protease inhibitor can be used to suppress production of the virus. NS3 protease inhibitors that can be used include, but are not limited to, simeprevir, danoprevir, asunaprevir, ciluprevir, boceprevir, sovaprevir, paritaprevir and telaprevir.

In another aspect, the present disclosure includes a recombinant virion comprising a conditionally replicating viral vector described herein.

In another aspect, the present disclosure includes a kit for preparing or using fusion proteins according to the methods described herein. Such kits may comprise one or more fusion proteins, nucleic acids encoding such fusion proteins, expression vectors, conditionally replicating viral vectors, cells, or other reagents for preparing or using fusion proteins, as described herein. The kit may further include a protease inhibitor, such as an HCV NS3 protease inhibitor, including, for example, simeprevir, danoprevir, asunaprevir, ciluprevir, boceprevir, sovaprevir, paritaprevir or telaprevir.

These and other embodiments of the subject present disclosure will readily occur to those of skill in the art in view of the disclosure herein.

### BRIEF DESCRIPTION OF THE DRAWINGS

These and other features, aspects, and advantages of the present disclosure will become better understood with regard to the following description, and accompanying drawings.

FIG. 1 depicts the normalized percentage CAR expression in cells transfected to express one of four different fusion proteins.

### DETAILED DESCRIPTION

The practice of the present disclosure will employ, unless otherwise indicated, conventional methods of molecular biology, chemistry, biochemistry, virology, and immunology, within the skill of the art. Such techniques are explained fully in the literature. See, e.g., Hepatitis C Viruses: Genomes and Molecular Biology (S. L. Tan ed., Taylor & Francis, 2006), Fundamental Virology, 3′ Edition, vol. I & II (B. N. Fields and D. M. Knipe, eds.); Handbook of Experimental Immunology, Vols. I-IV (D. M. Weir and C. C. Blackwell eds., Blackwell Scientific Publications); A. L Lehninger, Biochemistry (Worth Publishers, Inc, current addition); Sambrook, et al., Molecular Cloning: A Laboratory Manual (3^{rd} Edition, 2001), Methods In Enzymology (S. Colowick and N. Kaplan eds., Academic Press, Inc.).

All publications, patents and patent applications cited herein, whether supra or infra, are hereby incorporated by reference in their entireties.

### Definitions

In describing the present disclosure, the following terms will be employed, and are intended to be defined as indicated below.

It must be noted that, as used in this specification and the appended claims, the singular forms “a,” “an” and “the” include plural referents unless the content clearly dictates otherwise Thus, for example, reference to “a fusion protein” includes a mixture of two or more fusion proteins, and the like.

The term “about,” particularly in reference to a given quantity, is meant to encompass deviations of plus or minus five percent.

The term, “protease” as used herein, refers to a protease that can be inactivated by the presence or absence of a specific agent (e.g., that binds to the protease) In some embodiments, a protease is active (cleaves a cognate cleavage site) in the absence of the specific agent and is inactive (does not cleave a cognate cleavage site) in the presence of the specific agent. In some embodiments, the specific agent is a protease inhibitor. In some embodiments, the protease inhibitor specifically inhibits a given protease of the present disclosure.

Non-limiting examples of proteases include hepatitis C virus proteases (e.g., NS3 and NS2-3); signal peptidase; proprotein convertases of the subtilisin/kexin family (furin, PCI, PC2, PC4, PACE4, PC5, PC); proprotein convertases cleaving at hydrophobic residues (e.g., Leu, Phe, Val, or Met); proprotein convertases cleaving at small amino acid residues such as Ala or Thr; proopiomelanocortin converting enzyme (PCE); chromaffin granule aspartic protease (CGAP); prohormone thiol protease, carboxypeptidases (e.g., carboxypeptidase E/H, carboxypeptidase D and carboxypeptidase Z); aminopeptidases (e.g., arginine aminopeptidase, lysine aminopeptidase, aminopeptidase B); prolyl endopeptidase; aminopeptidase N, insulin degrading enzyme; calpain; high molecular weight protease; and, caspases 1, 2, 3, 4, 5, 6, 7, 8, and 9 Other proteases include, but are not limited to, aminopeptidase N; puromycin sensitive aminopeptidase; angiotensin converting enzyme; pyroglutamyl peptidase II; dipeptidyl peptidase IV; N-arginine dibasic convertase; endopeptidase 24.15; endopeptidase 24.16; amyloid precursor protein secretases alpha, beta and gamma, angiotensin converting enzyme secretase; TGF alpha secretase; T F alpha secretase; FAS ligand secretase; TNF receptor-I and -II secretases; CD30 secretase; KL1 and KL2 secretases; IL6 receptor secretase, CD43, CD44 secretase; CD 16-1 and CD 16-11 secretases; L-selectin secretase; Folate receptor secretase; MMP 1, 2, 3, 7, 8, 9, 10, 11, 12, 13, 14, and 15; urokinase plasminogen activator; tissue plasminogen activator; plasmin; thrombin; BMP-1 (procollagen C-peptidase); ADAM 1, 2, 3, 4, 5, 6, 7, 8, 9, 10, and 11; and, granzymes A, B, C, D, E, F, G, and H. For a discussion of proteases, see, e.g., V. Y. H. Hook, Proteolytic and cellular mechanisms in prohormone and proprotein processing, R G Landes Company, Austin, Tex., USA (1998); N. M. Hooper et al., Biochem. J. 321: 265-279 (1997); Z. Werb, Cell 91: 439-442 (1997); T. G. Wolfsberg et al., J. Cell Biol. 131: 275-278 (1995); K. Murakami and J. D. Etlinger, Biochem. Biophys. Res. Comm. 146: 1249-1259 (1987); T. Berg et al., Biochem. J. 307: 313-326 (1995); M. J. Smyth and J. A. Trapani, Immunology Today 16: 202-206 (1995), R V. Talanian et al., J. Biol. Chem. 272: 9677-9682 (1997); and N. A Thomberry et al., J. Biol. Chem. 272: 17907-17911 (1997), the disclosures of which are incorporated herein.

A “nonstructural protein 3 (NS3)” nucleic acid, oligonucleotide, protein, polypeptide, or peptide refers to a molecule derived from hepatitis C virus (HCV), including any isolate of HCV having any genotype (e.g., seven genotypes 1-7) or subtype. The molecule need not be physically derived from HCV, but may be synthetically or recombinantly produced A number of NS3 nucleic acid and protein sequences are known. Representative sequences are listed in the National Center for Biotechnology Information (NCBI) database. See, for example, NCBI entries: Accession Nos. YP_001491553, YP_001469631, YP_001469632, NP_803144, NP_671491, YP_001469634, YP_001469630, YP_001469633, ADA68311, ADA68307, AFP99000, AFP98987, ADA68322, AFP99033, ADA68330, AFP99056, AFP99041, CBF60982, CBF60817, AHH29575, AIZ00747, AIZ00744, ABI36969, ABN05226, KF516075, KF516074, KF516056, AB826684, AB826683, JX171009, JX171008, JX171000, EU847455, EF154714, GU085487, JX171065, JX171063; all of which sequences (as entered by the date of filing of this application) are herein incorporated by reference. Any of these sequences or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto, can be used to construct a fusion protein or a recombinant polynucleotide encoding such a fusion protein, as described herein.

A “nonstructural protein 4A (NS4A)” nucleic acid, oligonucleotide, protein, polypeptide, or peptide refers to a molecule derived from HCV, including any isolate of HCV having any genotype (e.g., seven genotypes 1-7) or subtype. The molecule need not be physically derived from HCV, but may be synthetically or recombinantly produced. A number of NS4A nucleic acid and protein sequences are known. Representative sequences are listed in the National Center for Biotechnology Information (NCBI) database. See, for example, NCBI entries: Accession Nos. NP_751925, YP_001491554, GU945462, HQ822054, FJ932208, FJ932207, FJ932205, and FJ932199; all of which sequences (as entered by the date of filing of this application) are herein incorporated by reference. Any of these sequences or a variant thereof comprising a sequence having at least about 80-100%) sequence identity thereto, including any percent identity within this range, such as 81, 82, 83, 84, 85, 86, 87, 88, 89, 90, 91, 92, 93, 94, 95, 96, 97, 98, or 99% sequence identity thereto, can be used to construct a fusion protein or a recombinant polynucleotide encoding such a fusion protein, as described herein.

A “polyprotein” nucleic acid, oligonucleotide, protein, polypeptide, or peptide refers to a molecule derived from HCV, including any isolate of HCV having any genotype (e.g., seven genotypes 1-7) or subtype. The molecule need not be physically derived from HCV, but may be synthetically or recombinantly produced. A number of polyprotein nucleic acid and protein sequences are known Representative HCV polyprotein sequences are listed in the National Center for Biotechnology Information (NCBI) database. See, for example, NCBI entries: Accession Nos. YP_001469631, NP 671491, YP_001469633, YP_001469630, YP_001469634, YP_001469632, NC_009824, NC 004102, NC_009825, NC_009827, NC_009823, NC_009826, and EF 108306; all of which sequences (as entered by the date of filing of this application) are herein incorporated by reference. Any of these sequences or a variant thereof comprising a sequence having at least about 80-100%) sequence identity thereto, including any percent identity within this range, such as 81, 82, 83, 84, 85, 86, 87, 88, 89, 90, 91, 92, 93, 94, 95, 96, 97, 98, or 99% sequence identity thereto, can be used to construct a fusion protein or a recombinant polynucleotide encoding such a fusion protein, as described herein.

For a discussion of genetic diversity and phylogenetic analysis of hepatitis C virus, see also Smith et al. (2014) Hepatology 59(1):318-327, Simmonds et al. (2005) Hepatology 42(4):962-973, Kuiken et al. (2009) Methods Mol Biol. 510.33-53, Ho et al. (2015) J. Virol. Methods 219:28-37, Echeverria et al. (2015) World J. Hepatol. 7(6):831-845, and Jackowiak et al (2014) Infect Genet Evol. 21:67-82; herein incorporated by reference in their entireties.

The terms “fusion protein,” “fusion polypeptide,” “degron fusion protein,” or “degron fusion” as used herein refer to a fusion comprising a degron in combination with a protease and a selected polypeptide of interest as part of a single continuous chain of amino acids, which chain does not occur in nature. The degron may be connected to the polypeptide of interest through a cleavable linker comprising a cleavage site capable of being recognized by the protease of the fusion to allow self-removal of the protease and degron from the polypeptide of interest. The position of the cleavage site in the fusion may be chosen to allow release of the polypeptide of interest from the fusion essentially unmodified or with little modification (e.g., less than 10 extra amino acids). The fusion polypeptides may be designed for N-terminal or C-terminal attachment of the degron to the polypeptide of interest. The fusion polypeptides may also contain sequences exogenous to the degron, protease, and polypeptide of interest. For example, the fusion may include targeting or localization sequences, detectable labels, or tag sequences.

The term, “cell receptor” as used herein, refers to a membrane protein that responds specifically to individual extracellular stimuli and generates intracellular signals that give rise to a particular functional responses. Non-limiting examples of these stimuli/signals include soluble factors generated locally (for example, synaptic transmission) or distantly (for example, hormones and growth factors), ligands on the surface of other cells (e.g., an antigen, such as a cancer antigen), or the extracellular matrix itself. Non-limiting examples of cell receptors include G protein coupled receptors, receptor tyrosine kinases, ligand gated ion channels, integrins, cytokine receptors, and chimeric antigen receptors (CARs).

The term, “chimeric antigen receptor” or alternatively a “CAR” as used herein refers to a polypeptide or a set of polypeptides, which when expressed in an immune effector cell, provides the cell with specificity for a target cell, typically a cancer cell, and with intracellular signal generation. In some embodiments, a CAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule. In some aspects, the set of polypeptides are contiguous with each other. In some embodiments, the CAR further comprises a spacer domain between the extracellular antigen binding domain and the transmembrane domain. In some embodiments, the set of polypeptides include recruitment domains, such as dimerization or multimerization domains, that can couple the polypeptides to one another. In some embodiments, the CAR comprises a chimeric fusion protein comprising an extracellular antigen binding domain, a transmembrane domain and an intracellular signaling domain comprising a functional signaling domain derived from a stimulatory molecule. In one aspect, the CAR comprises a chimeric fusion protein comprising an extracellular antigen binding domain, a transmembrane domain and an intracellular signaling domain comprising a functional signaling domain derived from a costimulatory molecule and a functional signaling domain derived from a stimulatory molecule. In one aspect, the CAR comprises a chimeric fusion protein comprising an extracellular antigen binding domain, a transmembrane domain and an intracellular signaling domain comprising two functional signaling domains derived from one or more costimulatory molecule(s) and a functional signaling domain derived from a stimulatory molecule. In some embodiments, the CAR comprises a chimeric fusion protein comprising an extracellular antigen binding domain, a transmembrane domain and an intracellular signaling domain comprising at least two functional signaling domains derived from one or more costimulatory molecule(s) and a functional signaling domain derived from a stimulatory molecule.

The term, “extracellular protein binding domain” as used herein, refers to a molecular binding domain which is typically an ectodomain of a cell receptor and is located outside the cell, exposed to the extracellular space. Am extracellular protein binding domain can include any molecule (e.g., protein or peptide) capable of binding to another protein or peptide. In some embodiments, an extracellular protein binding domain comprises an antibody, an antigen-binding fragment thereof, F(ab), F(ab′), a single chain variable fragment (scFv), or a single-domain antibody (sdAb). In some embodiments, an extracellular protein binding domain binds to a hormone, a growth factor, a cell-surface ligand (e.g., an antigen, such as a cancer antigen), or the extracellular matrix.

The term, “intracellular signaling domain” as used herein, refers to a functional endodomain of a cell receptor located inside the cell. Following binding of the molecular binding domain to an antigen, for example, the signaling domain transmits a signal (e.g., proliferative/survival signal) to the cell. In some embodiments, the signaling domain is a CD3-zeta protein, which includes three immunoreceptor tyrosine-based activation motifs (ITAMs) Other examples of signaling domains include CD28, 4-1BB, and OX40. In some embodiments, a cell receptor comprises more than one signaling domain, each referred to as a co-signaling domain.

The term, “transmembrane domain” as used herein, refers to a domain that spans a cellular membrane. In some embodiments, a transmembrane domain comprises a hydrophobic alpha helix. Different transmembrane domains result in different receptor stability. In some embodiments, a transmembrane domain of a cell receptor of the present disclosure comprises a CD3-zeta transmembrane domain or a CD28 transmembrane domain.

The term, “recruitment domain” as used herein, refers to an interaction motif found in various proteins, such as helicases, kinases, mitochondrial proteins, caspases, other cytoplasmic factors, etc. The recruitment domains mediate formation of a large protein complex via direct interactions between recruitment domains. In some embodiments, recruitment domains of the present disclosure are dimerization or multimerization domains.

The term, “cell-based therapy” as used herein, refers to a therapeutic method using cells (e.g., immune cells and/or stem cells) to deliver to a patient (a subject) a gene or polypeptide of interest, such as a therapeutic protein Cell based-therapies, as provided herein, also encompass preventative and diagnostic regimes. Thus, a gene of interest (and encoded product of interest) used in a cell-based therapy may be a prophylactic molecule (e.g., an antigen intended to induce an immune response) or a detectable molecule (e.g., a fluorescent protein or other visible molecule).

The term, “cognate cleavage site” as used herein, refers to a specific sequence or sequence motif recognized by and cleaved by a protease of the present disclosure. A cleavage site for a protease includes the specific amino acid sequence or motif recognized by the protease during proteolytic cleavage and typically includes the surrounding one to six amino acids on either side of the scissile bond, which bind to the active site of the protease and are used for recognition as a substrate.

The term “cleavable linker” refers to a linker comprising a cleavage site. The cleavable linker may include a cleavage site specific for an enzyme, such as a protease or other cleavage agent A cleavable linker is typically cleavable under physiological conditions.

The term, “degron” as used herein, refers to a protein or a part thereof that is important in regulation of protein degradation rates. Various degrons known in the art, including but not limited to short amino acid sequences, structural motifs, and exposed amino acids, can be used in various embodiments of the present disclosure. Degrons identified from a variety of organisms can be used. In some embodiments, degrons of the present disclosure comprise a degradation sequence. In some embodiments, the degron is a self-excising degron. A self-excising degron is a degron that is fused to a polypeptide of interest such that a protease of the present disclosure is capable of cleaving the fusion protein containing the polypeptide of interest to separate the degron from the polypeptide of interest. The protease itself may or may not be removed from the fusion protein containing the polypeptide of interest following cleavage.

The term, “degradation sequence” as used herein, refers to a sequence that promotes degradation of an attached protein through either the proteasome or autophagy-lysosome pathways. In preferred embodiments, a degradation sequence is a polypeptide that destabilize a protein such that half-life of the protein is reduced at least two-fold, when fused to the protein Many different degradation sequences/signals (e.g., of the ubiquitin-proteasome system) are known in the art, any of which may be used as provided herein A degradation sequence may be operably linked to a cell receptor, but need not be contiguous with it as long as the degradation sequence still functions to direct degradation of the cell receptor. In some embodiments, the degradation sequence induces rapid degradation of the cell receptor. For a discussion of degradation sequences and their function in protein degradation, see, e.g., Kanemaki et al. (2013) Pflugers Arch. 465(3):419-425, Erales et al. (2014) Biochim Biophys Acta 1843(1):216-221, Schrader et al. (2009) Nat. Chem. Biol. 5(11):815-822, Ravid et al. (2008) Nat. Rev. Mol. Cell. Biol. 9(9):679-690, Tasaki et al. (2007) Trends Biochem Sci 32(1l1):520-528, Meinnel et al. (2006) Biol. Chem. 387(7):839-851, Kim et al. (2013) Autophagy 9(7): 1100-1103, Varshavsky (2012) Methods Mol Biol 832, 1-11, and Fayadat et al. (2003) Mol Biol Cell 14(3): 1268-1278; herein incorporated by reference.

The terms “polypeptide” and “protein” refer to a polymer of amino acid residues and are not limited to a minimum length. Thus, peptides, oligopeptides, dimers, multimers, and the like, are included within the definition. Both full length proteins and fragments thereof are encompassed by the definition. The terms also include postexpression modifications of the polypeptide, for example, glycosylation, acetylation, phosphorylation, hydroxylation, and the like. Furthermore, for purposes of the present disclosure, a “polypeptide” refers to a protein which includes modifications, such as deletions, additions and substitutions to the native sequence, so long as the protein maintains the desired activity. These modifications may be deliberate, as through site directed mutagenesis, or may be accidental, such as through mutations of hosts which produce the proteins or errors due to PCR amplification.

By “derivative” is intended any suitable modification of the native polypeptide of interest, of a fragment of the native polypeptide, or of their respective analogs, such as glycosylation, phosphorylation, polymer conjugation (such as with polyethylene glycol), or other addition of foreign moieties, as long as the desired biological activity of the native polypeptide is retained. Methods for making polypeptide fragments, analogs, and derivatives are generally available in the art.

By “fragment” is intended a molecule consisting of only a part of the intact full length sequence and structure. The fragment can include a C-terminal deletion an N-terminal deletion, and/or an internal deletion of the polypeptide. Active fragments of a particular protein or polypeptide will generally include at least about 5-10 contiguous amino acid residues of the full length molecule, preferably at least about 15-25 contiguous amino acid residues of the full length molecule, and most preferably at least about 20-50 or more contiguous amino acid residues of the full length molecule, or any integer between 5 amino acids and the full length sequence, provided that the fragment in question retains biological activity, such as catalytic activity, ligand binding activity, regulatory activity, degron protein degradation signaling, or fluorescence characteristics.

“Substantially purified” generally refers to isolation of a substance (compound, polynucleotide, protein, polypeptide, polypeptide composition) such that the substance comprises the majority percent of the sample in which it resides. Typically, in a sample, a substantially purified component comprises 50%, preferably 80%-85, more preferably 90-95% of the sample. Techniques for purifying polynucleotides and polypeptides of interest are well-known in the art and include, for example, ion-exchange chromatography, affinity chromatography and sedimentation according to density.

By “isolated” is meant, when referring to a polypeptide, that the indicated molecule is separate and discrete from the whole organism with which the molecule is found in nature or is present in the substantial absence of other biological macro molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

The terms “label” and “detectable label” refer to a molecule capable of detection, including, but not limited to, radioactive isotopes, stable (non-radioactive) heavy isotopes, fluorescers, chemiluminescers, enzymes, enzyme substrates, enzyme cofactors, enzyme inhibitors, chromophores, dyes, metal ions, metal sols, ligands (e.g., biotin or haptens) and the like. The term “fluorescer” refers to a substance or a portion thereof that is capable of exhibiting fluorescence in the detectable range. Particular examples of labels that may be used with the present disclosure include, but are not limited to radiolabels (e.g., H, I, S, C, or P), stable (non-radioactive) heavy isotopes (e.g., ¹³C or ¹⁵N), phycoerythrin, Alexa dyes, fluorescein, 7-nitrobenzo-2-oxa-1,3-diazole (NBD), YPet, CyPet, Cascade blue, allophycocyanin, Cy3, Cy5, Cy7, rhodamine, dansyl, umbelliferone, Texas red, luminol, acradimum esters, biotin or other streptavidin-binding proteins, magnetic beads, electron dense reagents, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), Dronpa, Padron, mApple, mCherry, rsCherry, rsCherryRev, firefly luciferase, Renilla luciferase, NADPH, beta-galactosidase, horseradish peroxidase, glucose oxidase, alkaline phosphatase, chloramphenical acetyl transferase, and urease Enzyme tags are used with their cognate substrate. The terms also include color-coded microspheres of known fluorescent light intensities (see e.g., microspheres with xMAP technology produced by Luminex (Austin, Tex.); microspheres containing quantum dot nanocrystals, for example, containing different ratios and combinations of quantum dot colors (e.g., Qdot nanocrystals produced by Life Technologies (Carlsbad, Calif.); glass coated metal nanoparticles (see e.g., SERS nanotags produced by Nanoplex Technologies, Inc. (Mountain View, Calif.); barcode materials (see e.g., sub-micron sized striped metallic rods such as Nanobarcodes produced by Nanoplex Technologies, Inc.), encoded microparticles with colored bar codes (see e.g., CellCard produced by Vitra Bioscience, vitrabio.com), and glass microparticles with digital holographic code images (see e.g., CyVera microbeads produced by Ulumina (San Diego, Calif.). As with many of the standard procedures associated with the practice of the present disclosure, skilled artisans will be aware of additional labels that can be used.

“Homology” refers to the percent identity between two polynucleotide or two polypeptide molecules. Two nucleic acid, or two polypeptide sequences are “substantially homologous” to each other when the sequences exhibit at least about 50% sequence identity, preferably at least about 75% sequence identity, more preferably at least about 80%-85% sequence identity, more preferably at least about 90% sequence identity, and most preferably at least about 95%-98%, sequence identity over a defined length of the molecules. As used herein, substantially homologous also refers to sequences showing complete identity to the specified sequence.

In general, “identity” refers to an exact nucleotide to nucleotide or amino acid to amino acid correspondence of two polynucleotides or polypeptide sequences, respectively Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M. O. in Atlas of Protein Sequence and Structure M.O. Dayhoff ed., 5 Suppl. 3:353 358, National biomedical Research Foundation, Washington, D.C., which adapts the local homology algorithm of Smith and Waterman Advances in Appl. Math 2.482 489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity in the context of the present disclosure is to use the MPSRCH package of programs copyrighted by the University of Edinburgh, developed by John F. Collins and Shane S. Sturrok, and distributed by IntelliGenetics, Inc. (Mountain View, Calif.). From this suite of packages, the Smith Waterman algorithm can be employed where default parameters are used for the scoring table (for example, gap open penalty of 12, gap extension penalty of one, and a gap of six). From the data generated the “Match” value reflects “sequence identity” Other suitable programs for calculating the percent identity or similarity between sequences are generally known in the art, for example, another alignment program is BLAST, used with default parameters. For example, BLASTN and BLASTP can be used using the following default parameters genetic code:=standard, filter:=none; strand=both; cutoff=60; expect=10; Matrix=BLOSUM62; Descriptions=50 sequences; sort by=HIGH SCORE; Databases=non redundant, GenBank+EMBL+DDBJ+PDB+GenBank CDS translations+Swiss protein+Spupdate+PIR. Details of these programs are readily available.

Alternatively, homology can be determined by hybridization of polynucleotides under conditions which form stable duplexes between homologous regions, followed by digestion with single stranded specific nuclease(s), and size determination of the digested fragments. DNA sequences that are substantially homologous can be identified in a Southern hybridization experiment under, for example, stringent conditions, as defined for that particular system Defining appropriate hybridization conditions is within the skill of the art. See, e.g., Sambrook et al., supra; DNA Cloning, supra; Nucleic Acid Hybridization, supra.

“Recombinant” as used herein to describe a nucleic acid molecule means a polynucleotide of genomic, cDNA, viral, semisynthetic, or synthetic origin which, by virtue of its origin or manipulation, is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

The term “transformation” refers to the insertion of an exogenous polynucleotide into a host cell, irrespective of the method used for the insertion. For example, direct uptake, transduction or f-mating are included. The exogenous polynucleotide may be maintained as a non-integrated vector, for example, a plasmid, or alternatively, may be integrated into the host genome.

“Recombinant host cells,” “host cells,” “cells,” “cell lines,” “cell cultures,” and other such terms denoting microorganisms or higher eukaryotic cell lines, refer to cells which can be, or have been, used as recipients for a recombinant vector or other transferred DNA, and include the progeny of the cell which has been transfected. Host cells may be cultured as unicellular or multicellular entities (e.g., tissue, organs, or organoids comprising the recombinant vector).

A “coding sequence” or a sequence that “encodes” a selected polypeptide is a nucleic acid molecule that is transcribed (in the case of DNA) and translated (in the case of mRNA) into a polypeptide in vivo when placed under the control of appropriate regulatory sequences (or “control elements”). The boundaries of the coding sequence can be determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral or prokaryotic DNA, and even synthetic DNA sequences A transcription termination sequence may be located 3′ to the coding sequence.

Typical “control elements,” include, but are not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences.

“Operably linked” refers to an arrangement of elements wherein the components so described are configured so as to perform their usual function. For example, a given promoter operably linked to a coding sequence is capable of effecting the expression of the coding sequence when the proper enzymes are present. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence. In another example, a degron operably linked to a polypeptide is capable of promoting degradation of the polypeptide when the proper cellular degradation system (e.g., proteasome or autophagosome degradation) is present. The degron need not be contiguous with the polypeptide, so long as it functions to direct degradation of the polypeptide.

“Encoded by” refers to a nucleic acid sequence which codes for a polypeptide sequence, wherein the polypeptide sequence or a portion thereof contains an amino acid sequence of at least 3 to 5 amino acids, more preferably at least 8 to 10 amino acids, and even more preferably at least 15 to 20 amino acids from a polypeptide encoded by the nucleic acid sequence.

“Expression cassette” or “expression construct” refers to an assembly which is capable of directing the expression of the sequence(s) or gene(s) of interest. An expression cassette generally includes control elements, as described above, such as a promoter which is operably linked to (so as to direct transcription of) the sequence(s) or gene(s) of interest, and often includes a polyadenylation sequence as well. Within certain embodiments of the present disclosure, the expression cassette described herein may be contained within a plasmid construct. In addition to the components of the expression cassette, the plasmid construct may also include, one or more selectable markers, a signal which allows the plasmid construct to exist as single stranded DNA (e.g., a M1 3 origin of replication), at least one multiple cloning site, and a “mammalian” origin of replication (e.g., a SV40 or adenovirus origin of replication).

“Purified polynucleotide” refers to a polynucleotide of interest or fragment thereof which is essentially free, e.g., contains less than about 50%, preferably less than about 70%, and more preferably less than about at least 90%, of the protein with which the polynucleotide is naturally associated. Techniques for purifying polynucleotides of interest are well-known in the art and include, for example, disruption of the cell containing the polynucleotide with a chaotropic agent and separation of the polynucleotide(s) and proteins by ion-exchange chromatography, affinity chromatography and sedimentation according to density.

The term “transfection” is used to refer to the uptake of foreign DNA by a cell. A cell has been “transfected” when exogenous DNA has been introduced inside the cell membrane A number of transfection techniques are generally known in the art. See, e.g., Graham et al. (1973) Virology, 52:456, Sambrook et al. (2001) Molecular Cloning, a laboratory manual, 3^{r} edition, Cold Spring Harbor Laboratories, New York, Davis et al. (1995) Basic Methods in Molecular Biology, 2nd edition, McGraw-Hill, and Chu et al. (1981) Gene 13: 197. Such techniques can be used to introduce one or more exogenous DNA moieties into suitable host cells. The term refers to both stable and transient uptake of the genetic material, and includes uptake of peptide- or antibody-linked DNAs.

A “vector” is capable of transferring nucleic acid sequences to target cells (e.g., viral vectors, non-viral vectors, particulate carriers, and liposomes). Typically, “vector construct.” “expression vector,” and “gene transfer vector,” mean any nucleic acid construct capable of directing the expression of a nucleic acid of interest and which can transfer nucleic acid sequences to target cells. Thus, the term includes cloning and expression vehicles, as well as viral vectors.

The terms “variant,” “analog” and “mutein” refer to biologically active derivatives of the reference molecule that retain desired activity, such as fluorescence or oligomerization characteristics. In general, the terms “variant” and “analog” refer to compounds having a native polypeptide sequence and structure with one or more amino acid additions, substitutions (generally conservative in nature) and/or deletions, relative to the native molecule, so long as the modifications do not destroy biological activity and which are “substantially homologous” to the reference molecule as defined below. In general, the amino acid sequences of such analogs will have a high degree of sequence homology to the reference sequence, e.g., amino acid sequence homology of more than 50%, generally more than 60%-70%, even more particularly 80%-85% or more, such as at least 90%-95% or more, when the two sequences are aligned. Often, the analogs will include the same number of amino acids but will include substitutions, as explained herein. The term “mutein” further includes polypeptides having one or more amino acid-like molecules including but not limited to compounds comprising only amino and/or imino molecules, polypeptides containing one or more analogs of an amino acid (including, for example, unnatural amino acids, etc.), polypeptides with substituted linkages, as well as other modifications known in the art, both naturally occurring and non-naturally occurring (e.g., synthetic), cyclized, branched molecules and the like. The term also includes molecules comprising one or more N-substituted glycine residues (a “peptoid”) and other synthetic amino acids or peptides. (See, e.g., U.S. Pat. Nos. 5,831,005; 5,877,278; and 5,977,301; Nguyen et al., Chem. Biol. (2000) 7:463-473, and Simon et al., Proc. Natl. Acad. Sci. USA (1992) 89:9367-9371 for descriptions of peptoids). Methods for making polypeptide analogs and muteins are known in the art and are described further below.

As explained above, analogs generally include substitutions that are conservative in nature, i.e., those substitutions that take place within a family of amino acids that are related in their side chains. Specifically, amino acids are generally divided into four families: (1) acidic-aspartate and glutamate; (2) basic—lysine, arginine, histidine; (3) non-polar—alanine, valine, leucine, isoleucine, proline, phenylalanine, methionine, tryptophan; and (4) uncharged polar-glycine, asparagine, glutamine, cysteine, serine threonine, and tyrosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity. For example, the polypeptide of interest may include up to about 5-10 conservative or non-conservative amino acid substitutions, or even up to about 15-25 conservative or non-conservative amino acid substitutions, or any integer between 5-25, so long as the desired function of the molecule remains intact. One of skill in the art may readily determine regions of the molecule of interest that can tolerate change by reference to Hopp/Woods and Kyte-Doolittle plots, well known in the art.

“Gene transfer” or “gene delivery” refers to methods or systems for reliably inserting DNA or RNA of interest into a host cell. Such methods can result in transient expression of non-integrated transferred DNA, extrachromosomal replication and expression of transferred replicons (e.g., episomes), or integration of transferred genetic material into the genomic DNA of host cells. Gene delivery expression vectors include, but are not limited to, vectors derived from bacterial plasmid vectors, viral vectors, non-viral vectors, alphaviruses, pox viruses and vaccinia viruses.

The term “derived from” is used herein to identify the original source of a molecule but is not meant to limit the method by which the molecule is made which can be, for example, by chemical synthesis or recombinant means.

A polynucleotide “derived from” a designated sequence refers to a polynucleotide sequence which comprises a contiguous sequence of approximately at least about 6 nucleotides, preferably at least about 8 nucleotides, more preferably at least about 10-12 nucleotides, and even more preferably at least about 15-20 nucleotides corresponding, i.e., identical or complementary to, a region of the designated nucleotide sequence. The derived polynucleotide will not necessarily be derived physically from the nucleotide sequence of interest, but may be generated in any manner, including, but not limited to, chemical synthesis, replication, reverse transcription or transcription, which is based on the information provided by the sequence of bases in the region(s) from which the polynucleotide is derived. As such, it may represent either a sense or an antisense orientation of the original polynucleotide.

The term “heterologous” as it relates to nucleic acid sequences such as coding sequences and control sequences, denotes sequences that are not normally joined together, and/or are not normally associated with a particular cell. Thus, a “heterologous” region of a nucleic acid construct or a vector is a segment of nucleic acid within or attached to another nucleic acid molecule that is not found in association with the other molecule in nature. For example, a heterologous region of a nucleic acid construct could include a coding sequence flanked by sequences not found in association with the coding sequence in nature. Another example of a heterologous coding sequence is a construct where the coding sequence itself is not found in nature (e.g., synthetic sequences having codons different from the native gene). Similarly, a cell transformed with a construct which is not normally present in the cell would be considered heterologous for purposes of the present disclosure.

By “recombinant virus” is meant a virus that has been genetically altered, e.g., by the addition or insertion of a heterologous nucleic acid construct into the particle.

“Recombinant virion,” as used herein, refers to a viral particle containing a recombinant viral vector (e.g., conditionally replicating viral vector encoding a degron fusion protein). Generally, a recombinant virion comprises one or more structural proteins and the viral vector. The recombinant virion may also contain a nucleocapsid structure, and in some cases, a lipid envelope derived from the host cell membrane.

The terms “subject” refers to any invertebrate or vertebrate subject, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered.

“Recombinant animal” refers to a nonhuman subject which has been a recipient of a recombinant vector or other transferred DNA, and also includes the progeny of a recombinant

$$animal .$$

### Other Interpretational Conventions

Ranges recited herein are understood to be shorthand for all of the values within the range, inclusive of the recited endpoints. For example, a range of 1 to 50 is understood to include any number, combination of numbers, or sub-range from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless otherwise indicated, reference to a compound that has one or more stereocenters intends each stereoisomer, and all combinations of stereoisomers, thereof.

### Overview

Before describing the present disclosure in detail, it is to be understood that the present disclosure is not limited to particular formulations or process parameters as such may, of course, vary. It is also to be understood that the terminology used herein is for the purpose of describing particular embodiments of the present disclosure only, and is not intended to be limiting.

Although a number of methods and materials similar or equivalent to those described herein can be used in the practice of the present disclosure, the preferred materials and methods are described herein.

The present disclosure is based on the discovery that certain mutations within an immunodominant epitope of a protease of the present disclosure, such as the hepatitis C virus (HCV) nonstructural protein 3 (NS3) protease, can affect not only the immunogenicity, but also the activity of the protease when fused to a polypeptide of interest. Such mutations may be used to reduce the immunogenicity and modulate the activity of the protease when used in therapeutic applications, such as with small molecule-assisted shutoff (SMASh) techniques, in which a polypeptide of interest is fused to a and thereby expressed in a minimally modified form. In such applications, the degron can be removed from the protein of interest by a cis-encoded protease (e.g., a viral NS3 protease). Clinically available protease inhibitors can be used to block protease cleavage such that the degron is retained after inhibitor addition on subsequently synthesized protein copies. The degron when attached causes rapid degradation of the linked protein. Alternatively, a protease of the present disclosure may be fused to a polypeptide of interest with a functional domain, or in the case of a multi-domain polypeptide between domains such that addition of a protease inhibitor can control one or more functions of the polypeptide of interest. As disclosed herein use of such a repressible protease allows for reversible and dose-dependent shutoff of various proteins with high dynamic range in multiple cell types.

### Fusion Proteins

Certain aspects of the present disclosure relate to fusion proteins comprise a variant protease (e.g., a variant HCV NS3 protease) fused to a selected polypeptide of interest and a cognate protease cleavage site in an arrangement designed to control function and/or production of the polypeptide of interest. The cleavage site is capable of being recognized by the protease of the fusion protein in order to allow cleavage of one or more domains within the polypeptide of interest. The position of the cleavage site in the fusion is preferably chosen to allow for controlled function and/or expression of the polypeptide of interest. The fusion proteins of the present disclosure may be designed with N-terminal or C-terminal attachment of the protease to the polypeptide of interest. The fusion protein may also contain sequences exogenous to the protease, cognate cleavage site, and polypeptide of interest. For example, the fusion may include targeting or localization sequences, or tag sequences. In addition, the fusion protein may comprise a detectable label (e.g., fluorescent, bioluminescent, chemiluminescent, colorimetric, or isotopic label) to facilitate monitoring production and degradation of the polypeptide of interest.

### Variant Proteases

Certain aspects of the present disclosure relate to a fusion protein comprising a variant protease, wherein the variant protease comprises one or more mutations the decrease immunogenicity and/or modulate protease activity when the fusion protein is expressed in a mammalian cell.

Variant proteases of the present disclosure may be derived from any suitable protease known in the art. For example, any of the proteases listed in Table 1 may be used to produce a variant protease of the present disclosure. When a protease is selected, its cognate cleavage site and protease inhibitors known in the art to bind and inhibit the protease can be used in a combination. Exemplary combinations for the use are provided below in Table 1. Representative sequences of the proteases are available from public database including UniProt through the uniprot.org website. UniProt accession numbers for the proteases are also provided below in Table 1.

TABLE 1

| | UniProt Accession | Cognate cleavage | Specific |
|---|---|---|---|
| Protease | Number/Sequence | site | Inhibitors |
| HCVNS3 | APITAYAQQTRGLLGCIITSLT | ADLEVVTSTWL | Simeprevir, |
| | GRDKNQVEGEVQIVSTATQTFL | (NS3/NS4A) | Danoprevir, |
| | ATCINGVCWAVYHGAGTRTIA | (SEQ ID NO: 8) | Asunaprevir, |
| | SPKGPVIQMYTNVDQDLVGWP | CMSADLEVVTSTW | Ciluprevir, |
| | APQGSRSLTPCTCGSSDLYLVT | VLVGGVL | Boceprevir, |
| | RHADVIPVRRRGDSRGSLLSPR | (NS3/NS4A) | Sovaprevir, |
| | PISYLKGSSGGPLLCPAGHAVG | (SEQ ID NO: 4) | Paritaprevir, |
| | LFRAAVCTRGVAKAVDFIPVE | DEMEECSQHL | Telaprevir, |
| | NLETTMRSPVFTD | (NS4A/NS4B) | Grazoprevir, |
| | (SEQ ID NO: 2) | (SEQ ID NO: 9) | Glecaprevir, |
| | APITAYAQQT RGLLGCIITS | YQEFDEMEECSQH | Voxiloprevir |
| | LTGRDKNQVE GEVQIVSTAA | LPYIEQG | |
| | QTFLATCING VCWTVYHGAG | (NS4A/NS4B) | |
| | TRTIASSKGP VIQMYTNVDQ | (SEQ ID NO: 5) | |
| | DLVGWPAPQG ARSLTPCTCG | ECTTPCSGSWL | |
| | SSDLYLVTRH ADVIPVRRRG | (NS4B/NS5A) | |
| | DGRGSLLSPR PISYLKGSSG | (SEQ ID NO: 10) | |
| | GPLLCPAGHA VGIFRAAVCT | WISSECTTPCSGSW | |
| | RGVAKAVDFI PVEGLETTMR | LRDIWD | |
| | SPVFSD (SEQ ID NO: 12) | (NS4B/NS5A) | |
| | | (SEQ ID NO: 6) | |
| | | EDVVPCSMG | |
| | | (NS5A/NS5B) | |
| | | (SEQ ID NO: 11) | |
| | | GADTEDWCCSMSYSW | |
| | | TGAL | |
| | | (NS5A/NS5B) | |
| | | (SEQ ID NO: 7) | |
| HIV-1 | PQVTLWQRPLVTIKIGGQLKEA | | Amprenavir, |
| protease | LLDTGADDTVLEEMSLPGRWK | | Atazanavir, |
| | PKMIGGIGGFIKVRQYDQILI | | Darunavir, |
| | EICGHKAIGTVLVGPTPVNII | | Fosamprenavir, |
| | GRNLLTQIGCTLNF | | Indinavir, |
| | (SEQ ID NO: 13) | | Lopinavir, |
| | | | Nelfmavir, |
| | | | Ritonavir, |
| | | | Saquinavir, |
| | | | Tipranavir |
| Signal | P67812, P15367, | preference of | |
| peptidase | P00804, P0803 | eukaryotic signal | |
| | | peptidase for | |
| | | cleavage after | |
| | | residue 20 (Xaa^{20↓}) of | |
| | | pre(Apro)apoA-II: Ala, | |
| | | Cys > Gly > Ser, Thr > | |
| | | Pro > Asn, Val, Ile, | |
| | | Leu, Tyr, His, Arg, | |
| | | Asp. | |
| proprotein | Q16549, Q8NBP7, | (R/K)-X-(hydrophobic)-X↓, | |
| convertases | Q92824, P29120, | where | |
| cleaving at | Q6UW60, P29122, | X is any amino acid | |
| hydrophobic | Q9QXV0 | | |
| residues | | | |
| (e.g., | | | |
| Leu, Phe, | | | |
| Val, | | | |
| or Met); | | | |
| proprotein | Q16549, Q8NBP7, Q92824, | K/R)-(X)n-(K/R)↓, | |
| convertases | P29120, Q6UW60, P29122 | where n is 0, | |
| cleaving at | | 2, 4 or 6 and X is | |
| small amino | | any amino acid | |
| acid | | | |
| residues | | | |
| such as Ala or | | | |
| Thr; | | | |
| proopiomelanoc | Q9UO77615, 0776133 | Cleavage at paired | |
| ortin converting | | basic residues | |
| enzyme (PCE); | | in certain prohormones, | |
| | | either | |
| | | between them, or | |
| | | on the carboxyl | |
| | | side | |
| chromaffin | | lends to cleave | |
| granule aspartic | | dipeptide bonds | |
| protease | | that have hydrophobic | |
| (CGAP); | | residues as | |
| | | well as a beta- | |
| | | methylene group | |
| prohormone | P07154, P07711, | | |
| thiol protease | P06797, P25975, | | |
| (cathepsin L1) | Q28944 | | |
| carboxypeptidases | Q9M099, P15169, | cleaves a peptide | |
| (e.g., | Q04609, P08819, | bond at the | |
| carboxvpeptidase | P08818, O77564, | carboxy-terminal | |
| E/H, | P70627, 035409, | (C-terminal) end | |
| carboxypeptidase | P07519, Q8VZU3, | of a protein or | |
| D and | P22792, P15087, | peptide | |
| carboxypeptidase | P16870, Q9JHH6, | | |
| Z); | Q96IY4, Q7L8A9 | | |
| aminopeptidases | | cleaves a peptide | |
| (e.g., arginine | | bond at the | |
| aminopeptidase, | | amino-terminal | |
| lysine | | (N-terminal) end | |
| aminopeptidase, | | of a protein or | |
| aminopeptidase | | peptide | |
| B); | | | |
| prolyl | Q12884, P48147, | Hydrolysis of Pro-\|-Xaa >> | |
| endopeptidase; | P97321, Q4J6C6, | Ala-\|-Xaa in oligopeptides. | |
| | | Release of an N-terminal | |
| | | dipeptide, Xaa-Yaa-\|-Zaa-, | |
| | | from a polypeptide, | |
| | | preferentially when | |
| | | Yaa is Pro. provided Zaa | |
| | | is neither Pro nor | |
| | | hydroxyproline. | |
| aminopeptidase | P97449, P15144, | Release of an N-terminal | |
| N; | P15145, P15684 | Amino acid, Xaa-\|-Yaa- | |
| | | from a peptide, | |
| | | amide or arylamide. | |
| | | Xaa is preferably Ala, | |
| | | but may be most | |
| | | amino acids including | |
| | | Pro (slow action). | |
| | | When a terminal | |
| | | hydrophobic residue | |
| | | is followed | |
| | | by a prolyl residue, | |
| | | the two may | |
| | | be released as an | |
| | | intact Xaa-Pro | |
| | | dipeptide | |
| insulin | P14735, P35559, | Degradation of insulin, | |
| degrading | Q9JHR7, | Glucagon and other | |
| enzyme; | P22817, Q24K02 | polypeptides. No action | |
| | | on proteins. | |
| | | Cleaves multiple short | |
| | | polypeptides that vary | |
| | | considerably in sequence | |
| calpain; | 008529, P17655, | No specific amino acid sequence | |
| | Q07009, Q27971, | is uniquely recognized by | |
| | P20807, P07384, | calpains. Amongst protein | |
| | O35350, O14815, | substrates, tertiary structure | |
| | P04632, Q9Y6Q1, | elements rather than primary | |
| | O15484, Q9HC96, | amino acid sequences appear to be | |
| | A6NHC0, Q9UMQ6 | responsible for directing cleavage | |
| | | to a specific substrate. Amongst | |
| | | peptide and small-molecule | |
| | | substrates, the most consistently | |
| | | reported specificity is for small, | |
| | | hydrophobic amino acids (e.g., | |
| | | leucine, valine and isoleucine) at | |
| | | the P2 position, and large | |
| | | hydrophobic amino acids (e.g., | |
| | | phenylalanine and ty rosine) at the | |
| | | P1 position. One fluorogenic | |
| | | calpain substrate is (EDANS-Glu- | |
| | | Pro-Lcu-Phe═Ala-Glu-Arg-Lys- | |
| | | DABCYL) | |
| | | (EDANSEPLFAERKDABCYL, | |
| | | SEQ ID NO: 14), with cleavage | |
| | | occurring at the Phe═Ala bond. | |
| caspase 1 | P29466, P29452 | Strict requirement for an Asp | |
| | | residue at position P1 and has a | |
| | | preferred cleavage sequence of | |
| | | Tyr-Val-Ala-Asp-\|- (YVAD, SEQ | |
| | | ID NO: 15). | |
| caspase 2 | P42575, P29594 | Strict requirement for an Asp | |
| | | residue at P1, with 316-asp being | |
| | | essential for proteolytic activity | |
| | | and has a preferred cleavage | |
| | | sequence of Val-Asp-Val-Ala- | |
| | | Asp-\|- (YDVAD, | |
| | | SEQ ID NO: 16) | |
| caspase 3 | P42574, P70677 | Strict requirement for an Asp | |
| | | residue at positions P1 and P4. It | |
| | | has a preferred cleavage sequence | |
| | | of Asp-Xaa-Xaa-Asp-\|- with a | |
| | | hydrophobic amino-acid residue at | |
| | | P2 and a hydrophilic amino-acid | |
| | | residue at P3, although Val or Ala | |
| | | are also accepted at this position. | |
| caspase 4 | P70343, P49662 | Strict requirement for Asp at the | |
| | | P1 position. It has a preferred | |
| | | cleavage sequence of Tyr-Val- | |
| | | Ala-Asp-\|- (YVAD, SEQ ID NO: | |
| | | 15) but also cleaves at Asp-Glu- | |
| | | Val-Asp-\|- (DEVD; SEQ ID NO: 17) | |
| caspase 5 | P51878 | Strict requirement for Asp at the | |
| | | P1 position. It has a preferred | |
| | | cleavage sequence of Tyr-Val- | |
| | | Ala-Asp-\|- (YVAD, SEQ ID NO: | |
| | | 15) but also cleaves at Asp-Glu- | |
| | | Val-Asp-\|- | |
| | | (DEVD; SEQ ID NO: 17). | |
| caspase 6 | P55212 | Strict requirement for Asp at | |
| | | position P1 and has a preferred | |
| | | cleavage sequence of Val-Glu- | |
| | | His-Asp-\|- (VEHD; SEQ ID NO: | |
| | | 18). | |
| caspase 7 | P97864, P55210 | Strict requirement for an Asp | |
| | | residue at position P1 and has a | |
| | | preferred cleavage sequence of | |
| | | Asp-Glu-Val-Asp-KDEVD; SEQ | |
| | | ID NO: 17). | |
| caspase 8 | Q8IRY7, 089110, | Strict requirement for Asp at | |
| | Q14790 | position P1 and has a preferred | |
| | | cleavage sequence of | |
| | | (Leu/Asp/Val)-Glu-Thr-Asp-\|- | |
| | | (Gly/Ser/Ala). | |
| caspase 9 | P55211, Q8C3Q9, | Strict requirement for an Asp | |
| | Q5IS54 | residue at position P1 and with a | |
| | | marked preference for His at | |
| | | position P2. It has a preferred | |
| | | cleavage sequence of Leu-Gly- | |
| | | His-Asp-\|-Xaa (LGHD (SEQ ID | |
| | | NO: 19) -\|- Xaa). | |
| caspase 10 | Q92851 | Strict requirement for Asp at | |
| | | position P1 and has a preferred | |
| | | cleavage sequence of Leu-Gln- | |
| | | Thr-Asp-\|-Gly (LQTDG, SEQ ID | |
| | | NO: 20). | |
| puromycin | P55786, Q11011, | Release of an N-terminal amino acid, | |
| sensitive | | preferentially alanine, from a | |
| aminopeptidase: | | wide range of peptides, amides | |
| | | and arvlamides. | |
| angiotensin | P12821, P09470, | Release of a C-terminal dipeptide, | Benazepril |
| converting | Q9BYF1 | oligopeptide-\|-Xaa-Yaa, when Xaa | (Lotensin), |
| enzyme (ACE); | MGAASGRRGP GLLLPLPLLL | is not Pro, and Yaa is neither Asp | Captopril, |
| | LLPPQPALAL DPGLQPGNFS | nor Glu. | Enalapril |
| | ADEAGAQLFA QSYNSSAEQV | | (Vasotec), |
| | LFQSVAASWA HDTNITAENA | | Fosinopril, |
| | RRQEEAALLS QEFAEAWGQK | | Lisinopril |
| | AKELYEPIWQ NFTDPQLRRI | | (Prinivil, |
| | IGAVRTLGSA NLPLAKRQQY | | Zestril), |
| | NALLSWMSRI YSTAKVCLPN | | Moexipril, |
| | KTATCWSLDP DLTNILASSR | | Perindopril |
| | SYAMLLFAWE GWHNAAGIPL | | (Aceon), |
| | KPLYEDFTAL SNEAYKQDGF | | Quinapril |
| | TDTGAYWRSW YNSPTFEDDL | | (Accupril), |
| | SHLYQQLEPL YLNLHAFVRR | | Ramipril |
| | ALHRRYGDRY INLRGPIPAH | | (Altace), |
| | LLGDMWAQSW ENIYDMVVPF | | Trandolapril |
| | PDKPNLDVTS TMLQQGWNAT | | (Mavik), |
| | HMFRVAEEFF TSLELSPMPP | | Zofenopril, |
| | EFWEGSMLEK PADGREVVCH | | |
| | ASAWDFYNRK DPRIKQCTRV | | |
| | TMDQLSTVHH EMGHIQYYLQ | | |
| | YKDLPVSLRR GANPGFHEAI | | |
| | GDYLALSVST PEHLHKIGLL | | |
| | DRVTNDTESD INYLLKMALE | | |
| | KIAFLPFGYL VDQWRWGVFS | | |
| | GRTPPSRYNF DWWYLRTKYQ | | |
| | GICPPVTRNE THFDAGAKFH | | |
| | VPNVTPYIRY FVSFVLQFQF | | |
| | HEALCKEAGY EGPLHQCDIY | | |
| | RSTKAGAKLR KVLQAGSSRP | | |
| | WQEVLKDMVG LDALDAQPLL | | |
| | KYFQPVTQWL QEQNQQNGEV | | |
| | LGWPEYQWHP PLPDNYPEGI | | |
| | DLVTDEAEAS KFVEEYDRTS | | |
| | QVVWNEYAEA NWNYNTNITT | | |
| | ETSKILLQKN MQIANHTLKY | | |
| | GTQARKFDVN QLQNTTIKRI | | |
| | IKKVQDLERA ALPAQELEEY | | |
| | NKILLDMETT YSVATVCHPN | | |
| | GSCLQLEPDL TNVMATSRKY | | |
| | SDLLWAWEGW RDKAGRAILQ | | |
| | FYPKYVELIN QAARLNGYVD | | |
| | AGDSWRSMYE TPSLEQDLER | | |
| | LFQELQPLYL NLHAYVRRAL | | |
| | HRHYGAQHIN LEGPIPAHLL | | |
| | GNMWAQTWSN IYDLVVPFPS | | |
| | APSMDTTSAM LKQGWTPRRM | | |
| | FKEADDFFTS LGLLPVPPEF | | |
| | WNKSMLEKPT DGREVVCHAS | | |
| | AWDFYNGKDF RIKQCTTVNL | | |
| | EDLVVAHHEM GHIQYFMQYK | | |
| | DLPVALREGA NPGFHSAIGD | | |
| | VLALSVSTPK HLHSLNLLSS | | |
| | EGGSDEHDIN FLMKMALDKI | | |
| | AFIPFSYLVD QWRWRVFDGS | | |
| | ITKENYNQEW WSLRLKYQGL | | |
| | CPPVPRTQGD FDPGAKFHIP | | |
| | SSVPYIRYFV SFIIQFQFHE | | |
| | ALCQAAGHTG PLHKCDIYQS | | |
| | KEAGQRLATA MKLGFSRPWP | | |
| | EAMQLITGQP NMSASAMLSY | | |
| | FKPLLDWLRT ENELHGEKLG | | |
| | WPQYNWTPNS ARSEGPLPDS | | |
| | GRVSFLGLDL DAQQARVGQW | | |
| | LLLFLGIALL VATLGLSQRL | | |
| | FSIRHRSLHR HSHGPQFGSE | | |
| | VELRHS | | |
| | (SEQ ID NO: 21) | | |
| pyroglutamyl | Q9NXJ5 | Release of the N-terminal | |
| peptidase II; | | pyroglutamyl group from pGlu-- | |
| | | His-Xaa tripeptides and pGlu-- | |
| | | His-Xaa-Gly tetrapeptides | |
| dipeptidyl | P27487, P14740, | Release of an N-terminal | |
| peptidase IV; | P28843 | dipeptide, Xaa-Yaa-\|-Zaa-, | |
| | | from a polypeptide, | |
| | | preferentially when | |
| | | Yaa is Pro, provided | |
| | | Zaa is neither | |
| | | Pro nor hydroxyproline. | |
| N-arginine | O43847, Q8BHG1 | Hydrolysis of polypeptides, | |
| dibasic | | preferably at -Xaa-\|-Arg-Lys-, | |
| convertase; | | And less commonly at | |
| | | -Arg-\|-Arg-Xaa-, in which | |
| | | Xaa is not Arg or Lys. | |
| endopeptidase | P52888, P24155 | Preferential cleavage of bonds | |
| 24.15 (thimet | | with hydrophobic residues at P1, | |
| oligopeptidase) | | P2 and P3′ and a small | |
| | | residue at P1′ in | |
| | | substrates of 5 to 15 | |
| | | residues. | |
| endopeptidase | Q9BYT8, Q91YP2 | Preferential cleavage in | |
| 24.16 | | neurotensin: 10-Pro-\|-Tyr-11 | |
| (neurolysin) | | | |
| amyloid | P05067, P12023, | Endopeptidase of broad | |
| precursor | Q9Y5Z0, P56817 | specificity. | |
| protein | | | |
| secretase | | | |
| alpha | | | |
| amyloid | P05067, P12023, | Broad endopeptidase specificity. | |
| precursor | Q9Y5Z0, P56817 | Cleaves Glu-Val-Asn-Leu-\|-Asp- | |
| protein | | Ala-Glu-Phe (EVNLDAEF, SEQ | |
| secretase | | ID NO: 22) in the | |
| beta | | Swedish variant of | |
| | | AlzhFeimer's amyloid | |
| | | precursor protein. | |
| amyloid | P05067, P12023, | intramembrane cleavage of | |
| precursor | Q9Y5Z0, P56817 | integral membrane proteins | |
| protein | | | |
| secretase | | | |
| gamma | | | |
| MMP 1 | P03956, Q9EPL5uy | Cleavage of the triple helix of | SB-3CT |
| | | collagen at about three-quarters of | p-OH |
| | | the length of the molecule from | SB-3CT |
| | | the N-terminus, at 775-Gly-\|-Ile- | O-phosphate |
| | | 776 in the alpha-1(I) chain. | |
| | | Cleaves synthetic substrates | |
| | | and alpha-macroglobulins at bonds | SB-3CT |
| | | where P1′ is a hydrophobic | RXP470.1 |
| | | residue. | |
| MMP 2 | P08253, P33434 | Cleavage of gelatin type I and | SB-3CT |
| | | collagen types IV, V, VII, X. | p-OH SB-3CT |
| | | Cleaves the collagen-like | O-phosphate |
| | | sequence Pro-Gln-Gly-\|-Ile-Ala- | SB-3CT |
| | | Gly-Gln (PQGIAGQ, SEQ ID | RXP470.1 |
| | | NO: 23). | |
| MMP 3 | P08254, P28862 | Preferential cleavage where P1′, | SB-3CT |
| | | P2′ and P3′ are hydrophobic | p-OH SB-3CT |
| | | residues. | O-phosphate |
| | | | SB-3CT |
| | | | RXP470.1 |
| MMP 7 | P09237, Q10738 | Cleavage of 14-Ala-\|-Leu-15 and | SB-3CT |
| | | 16-Tyr-\|-Leu-17 in B chain of | p-OH SB-3CT |
| | | insulin. No action on collagen | O-phosphate |
| | | types I, II, IV, V. Cleaves gelatin | SB-3CT |
| | | chain alpha-2(I) > alpha-1(1). | RXP470.1 |
| MMP 8 | P22894, O70138 | Can degrade fibrillar type I, II, | SB-3CT |
| | | and III collagens. | p-OH SB-3CT |
| | | Cleavage of interstitial collagens | O-phosphate |
| | | in the triple helical domain. | SB-3CT |
| | | Unlike EC 3.4.24.7, this enzyme | RXP470.1 |
| | | cleaves type III collagen more | |
| | | slowly than type I. | |
| MMP 9 | P14780, P41245 | Cleavage of gelatin ty pes I and V | SB-3CT |
| | | and collagen types IV and V. | p-OH SB-3CT |
| | | Cleaves KiSS1 at a Gly-\|-Leu | O-phosphate |
| | | bond. | SB-3CT |
| | | Cleaves type IV and type V | RXP470.1 |
| | | collagen into large C-terminal | |
| | | three quarter fragments and | |
| | | shorter N-tenninal one quarter | |
| | | fragments. Degrades fibronectin | |
| | | but not laminin or Pz-peptide. | |
| MMP 10 | P09238, O55123 | Can degrade fibroncctin, gelatins | SB-3CT |
| | | of type I, III, IV, and V; weakly | p-OH SB-3CT |
| | | collagens III, IV, and V. | O-phosphate |
| | | | SB-3CT |
| | | | RXP470.1 |
| MMP 11 | P24347, Q02853 | A(A/Q)(N/A)↓(L/Y) | SB-3CT |
| | | (T/V/M/R)(R/K) | p-OH SB-3CT |
| | | G(G/A)E1LR | O-phosphate |
| | | ↓ denotes the cleavage site | SB-3CT |
| | | | RXP470.1 |
| MMP 12 | P39900, P34960 | Hydrolysis of soluble and | SB-3CT |
| | | insoluble elastin. Specific | p-OH SB-3CT |
| | | cleavages arc also produced at 14- | O-phosphate |
| | | Ala-\|-Leu-15 and 16-Tyr-\|-Leu-17 | SB-3CT |
| | | in the B chain of insulin has | RXP470.1 |
| | | significant elastolytic activity. | |
| | | Can accept large and small amino | |
| | | acids at the P1′ site, but has a | |
| | | preference for leucine. Aromatic | |
| | | or hydrophobic residues are | |
| | | preferred at the P1 site, with small | |
| | | hydrophobic residues (preferably | |
| | | alanine) occupying P3. | |
| MMP 13 | P45452, P33435 | Cleaves triple helical collagens, | SB-3CT |
| | | including type I, type II and type | p-OH SB-3CT |
| | | III collagen, but has the highest | O-phosphate |
| | | activity with soluble type II | SB-3CT |
| | | collagen. Can also degrade | RXP470.1 |
| | | collagen type IV, type XIV and | |
| | | type X. | |
| MMP 14 | P50281, P53690 | Activates progclalinase A by | SB-3CT |
| | | cleavage of the propeptide at 37- | p-OH SB-3CT |
| | | Asn-\|-Leu-38. Other bonds | O-phosphate |
| | | hydrolyzed include 35-Gly-\|-Ile- | SB-3CT |
| | | 36 in the propeptide of | RXP470.1 |
| | | collagenase 3. and 341-Asn-\|-Phe- | |
| | | 342, 441-Asp-\|-Leu-442 and 354- | |
| | | Gln-\|-Thr-355 in the aggrecan | |
| | | interglobular domain. | |
| urokinase | P00749, P06869 | Specific cleavage of Arg-\|-Val | Plasminogen |
| plasminogen | | bond in plasminogen to form | activator |
| activator (uPA) | | plasmin. | inhibitors |
| | | | (PAI) |
| tissue | P00750, P11214 | Specific cleavage of Arg-\|-Val | Plasminogen |
| plasminogen | | bond in plasminogen to form | activator |
| activator (tPA) | | plasmin. | inhibitors |
| | | | (PAI) |
| plasmin | P00747, P20918 | Preferential cleavage: Lys-\|-Xaa > | α-2- |
| | | Arg-\|-Xaa, higher selectivity than | antiplasmin |
| | | trypsin. Converts fibrin into | (AP) |
| | | soluble products. | |
| thrombin | P00734, P19221 | Cleaves bonds after Arg and Lvs | |
| | | Converts fibrinogen to fibrin and | |
| | | activates factors V, VII, VIII, | |
| | | XIII, and, in complex with | |
| | | thrombomodulin, protein C. | |
| BMP-1 | P13497, P98063 | Cleavage of the C-terminal | |
| (procollagen C- | | propeptide at Ala-\|-Asp in type I | |
| peptidase) | | and II procollagens and at Arg-\|- | |
| | | Asp in type III. | |
| ADAM | Q9P0K1, Q9UKQ2, Q9JLN6, | | SB-3CT |
| | O14672, Q13444, P78536, | | p-OH SB-3CT |
| | Q13443, O43184, P78325, | | O-phosphatc |
| | Q9UKF5, Q9BZ11, Q9H2U9, | | SB-3CT |
| | Q99965, O75077, Q9H013, | | RXP470.1 |
| | O43506 | | |
| granzyme A | P12544, P11032 | Preferential cleavage: -Arg-\|-Xaa-, | |
| | | -Lys-\|-Xaa->>-Phe-\|-Xaa- in | |
| | | small molecule substrates. | |
| granzyme B | P10144, P04187 | Preferential cleavage: | |
| | | -Asp-\|-Xaa->>-Asn-\|-Xaa- > | |
| | | -Met-\|-Xaa-, -Ser-\|-Xaa-. | |
| granzyme C/ | P08882, P20718 | Preference for bulky and aromatic | |
| granzyme H | | residues at the P1 position and | |
| | | acidic residues at the P3′ | |
| | | and P4′ sites. | |
| granzyme M | P51124, Q03238 | Cleaves peptide substrates after | |
| | | methionine, leucine, and | |
| | | norleucine. | |
| tobacco Etch | P04517, P0CK09 | E-Xaa-Xaa-Y -Xaa-Q-(G/S), with | |
| virus (TEV) | | cleavage occurring between Q and | |
| protcase | | G/S. The most common sequence | |
| | | is ENLYFQS (SEQ ID NO: 24). | |
| chymotrypsin- | P08217, Q9UNI1, Q91X79, | | -Thermobifida |
| like serine | P08861, P09093, P08218 | | fusca |
| protease | | | Thermopin |
| | | | -Pyrobaculum |
| | | | aerophilum |
| | | | Aeropin |
| | | | -Thermococciis |
| | | | kodakaraensis |
| | | | Tk-serpin |
| | | | -Alteromonas |
| | | | sp. |
| | | | Marinostatin |
| | | | -Streptomyces |
| | | | misionensis |
| | | | SMTI |
| | | | -Streptomyces |
| | | | sp. |
| | | | chymostatin |
| alphavirus | P08411, P03317, P13886, | | |
| proteases | Q8JUX6, Q86924, Q4QXJ8, | | |
| | 08QL53, P27282, Q5XXP4 | | |
| chymotrypsin- | Q86TL0, Q14790, Q99538, | | -Thermobifida |
| like cysteine | O15553 | | fusca |
| proteases | | | Thermopin |
| | | | -Pyrobaculum |
| | | | aerophilum |
| | | | Aeropin |
| | | | -Thermococcus |
| | | | kodakaraensis |
| | | | Tk-serpin |
| | | | -Alteromonas |
| | | | sp. |
| | | | Marinostatin |
| | | | -Streptomyces |
| | | | misionensis |
| | | | SMTI |
| | | | -Streptomyces |
| | | | sp. |
| | | | chymostatin |
| papain-like | P25774, P53634, Q96K76 | | |
| cysteine | | | |
| protcascs | | | |
| picomavirus | P03305, P03311, P13899 | | |
| leader proteases | | | |
| HIV proteases | P04585, P03367, P04584, | | |
| | P03369, P12497, P03366, | | |
| | P04587 | | |
| Herpesvirus | P10220, Q2HRB6, O40922, | | |
| proteases | O69527 | | |
| adenovirus | P03252, P24937, Q83906, | | |
| proteases | P68985, P09569, P11825, | | |
| | P10381 | | |
| Streptomyces | P00776 | | |
| griseus protease | | | |
| A (SGPA) | | | |
| Streptomyces | P00777 | | |
| griseus protcase | | | |
| B (SGPB) | | | |
| alpha-lytic | P85142, P00778 | | |
| protease | | | |
| serine | P48740, P98064, Q9UL52, | | |
| proteases | P05981, O60235 | | |
| cysteine | Q86TL0, Q14790, Q8WYN0, | | |
| proteases | Q96DT6, P55211 | | |
| aspartic | Q9Y5Z0, P56817, Q00663, | | |
| protcascs | Q53RT3, P0CY27 | | |
| threonine | Q9UI38, Q16512, Q9H6P5, | | |
| proteases | Q8IWU2, | | |
| Mast cell (MC) | NM_001836 | Abz-HPFHL (SEQ ID NO: 25)- | BAY 1142524 |
| chymase | | Lys(Dnp)-NH2 (SEQ ID NO: 56) | SUN13834 |
| (CMA1) | | | |
| Rat mast cell | NM_017145, NM_172044, | Abz-HPFHL (SEQ ID NO: 25)- | TY-51469 |
| protcase | NM_001170466, | Lys(Dnp)-NH2 (SEQ ID NO: 56) | |
| −1,−2, | NM_019321, | | |
| −3, −4, −5 | NM_013092 | | |
| Rat vascular | O70500 | Abz-HPFHL | |
| chymase | | (SEQ ID NO: 25)- | |
| (RVCH) | | Lys(Dnp)-NH2 | |
| | | (SEQ ID NO: 56) | |
| DENV NS3pro | >sp\|P33478\|1475-2093 | A strong preference for basic | Anthraquinone |
| (NS2B/NS3) | SGVLWDTPSPPEVERAVLDDGI | amino acid residues (Arg/Lys) at | BP13944 |
| | YRIMQRGLLGRSQVGVGVFQD | the P1 positions was observed, | ZINC04321905 |
| | GVFHTMWHVTRGAVLMYQG | whereas the preferences for the | MB21 |
| | KRLEPSWASVKKDLISYGGGW | P2-4 sites were in the order of | Policresulen |
| | RFQGSWNTGEEVQVIAVEPGK | Arg > Thr > Gln/Asn/Lys for P2, | SK-12 |
| | NPKNVQTAPGTFKTPEGEVGAI | Lys > Arg > Asn for P3, and Nle > | NSC135618 |
| | ALDFKPGTSGSPIVNREGKIVG | Leu > Lys > Xaa for P4. The | Biliverdin |
| | LYGNGWTTSGTYVSAIAQAK | prime site substrate specificity | |
| | ASQEGPLPEIEDEVFRKRNLTI | was for small and polar amino | |
| | MDLHPGSGKTRRYLPAIVREAI | acids in P1 and P3. | |
| | RRNVRTLILAPTRVVASEMAE | | |
| | ALKGMPIRYQTTAVKSEHTGK | | |
| | EIVDLMCHATFTMRLLSPVRVP | | |
| | NYNMIIMDEAHFTDPASIARRG | | |
| | YISTRVGMGEAAAIFMTATPPG | | |
| | SVEAFPQSNAVIQDEERDIPERS | | |
| | WNSGYEWITDFPGKTVWFVPS | | |
| | IKSGNDIANCLRKNGKRVIQLS | | |
| | RKTFDTEYQKTKNNDWDYVV | | |
| | TTDISEMGANFRADRVIDPRRC | | |
| | LKPVILKDGPERVILAGPMPVT | | |
| | VASAAQRRGRIGRNQNKEGDQ | | |
| | YVYMGQPLNNDEDHAHWTEA | | |
| | KMLLDNINTPEGIIPALFEPERE | | |
| | KSAAIDGEYRLRGEARKTFVEL | | |
| | MRRGDLPVWLSYKVASEGFQ | | |
| | YSDRRWCFDGERNNQVLEEN | | |
| | MDVEMWTKEGERKKLRPRWL | | |
| | DARTYSDPLALREFKEFAAGR | | |
| | R (SEQ ID NO: 26) | | |
| | >sp\|P14340\|1476-2093 | | |
| | AGVLWDVPSPPPVGKAELEDG | | |
| | AYRIKQKGILGYSQIGAGVYKE | | |
| | GTFHTMWHVTRGAVLMHKGK | | |
| | RIEPSWADVKKDLISYGGGWK | | |
| | LEGEWKEGEEVQVLALEPGKN | | |
| | PRAVQTKPGLFKTNAGTIGAVS | | |
| | LDFSPGTSGSPIIDKXGKVVGL | | |
| | YGNGVVTRSGAYVSAIAQTEK | | |
| | SIEDNPEIEDDIFRKRKLTIMDL | | |
| | HPGAGKTKRYLPAIVREAIKRG | | |
| | LRTLILAPTRVVAAEMEEALRG | | |
| | LPIRYQTPAIRAEHTGREIVDL | | |
| | MCHATFTMRLLSPVRVPNYNL | | |
| | IIMDEAHFTDPASIAARGYISTR | | |
| | VEMGEAAGIFMTATPPGSRDPF | | |
| | PQSNAPIMDEEREIPERSWSSG | | |
| | HEWVTDFKGKTVWFVPSIKAG | | |
| | NDIAACLRKNGKKVIQLSRKTF | | |
| | DSEYVKTRTNDWDFVVTTDIS | | |
| | EMGANFKAERVIDPRRCMKPV | | |
| | ILTDGEERVILAGPMPVTHSSA | | |
| | AQRRGRIGRNPKNENDQYIYM | | |
| | GEPLENDEDCAHWKEAKMLLD | | |
| | NINTPEGIIPSMFEPEREKVDA | | |
| | IDGEYRLRGEARKTFVDLMRR | | |
| | GDLPVWLAYRVAAEGINYADR | | |
| | RWCFDGIKNNQILEENVEVEI | | |
| | WTKEGERKKLKPRWLDAKIYS | | |
| | DPLALKEFKEFAAGRK | | |
| | (SEQ ID NO: 27) | | |
| | >sp\|Q99D3511474-2092 | | |
| | SGVLWDVPSPPETQKAELEEG | | |
| | VYRIKQQGIFGKTQVGVGVQK | | |
| | EGVFHTMWHVTRGAVLTHNG | | |
| | KRLEPNWASVKKDLISYGGGW | | |
| | RLSAQWQKGEEVQVIAVEPGKN | | |
| | PKNFQTMPGIFQTTTGEIGAIA | | |
| | LDFKPGTSGSPIINREGKVVGL | | |
| | YGNGVVTKNGGYVSGIAQTNA | | |
| | EPDGPTPELEEEMFKKRNLTIM | | |
| | DLHPGSGKTRKYLPAIVREAIK | | |
| | RRLRTLILAPTRVVAAEMEEAL | | |
| | KGLPIRYQTTATKSEHTGREIV | | |
| | DLMCHATFTMRLLSPVRVPNYN | | |
| | LIIMDEAHFTDPASIAARGYIS | | |
| | TRVGMGEAAAIFMTATPPGTAD | | |
| | AFPQSNAPIQDEERDIPERSW | | |
| | NSGNEWITDFVGKTVWFVPSIK | | |
| | AGNDIANCLRKNGKKVIQLSR | | |
| | KTFDTEYQKTKLNDWDFWTTD | | |
| | ISEMGANFKADRVIDPRRCLK | | |
| | PVILTDGPERVILAGPMPVTVA | | |
| | SAAQRRGRVGRNPQKENDQYI | | |
| | FMGQPLNKDEDHAHWTEAKMLL | | |
| | DNINTPEGIIPALFEPEREKSA | | |
| | AIDGEYRLKGESRKTFVELMR | | |
| | RGDLPVWLAHKVASEGIKYTD | | |
| | RKWCFDGERNNQILEENMDVE | | |
| | IWTKEGEKKKLRPRWLDARTY | | |
| | SDPLALKEFKDFAAGRK | | |
| | (SEQ ID NO: 28) | | |
| | >sp\|Q5UCB8\|1475-2092 | | |
| | SGALWDVPSPAATQKAALSEG | | |
| | VYRIMQRGLFGKTQVGVGIHIE | | |
| | GVFHTMWHVTRGSVICHETGR | | |
| | LEPSWADVRNDMISYGGGWR | | |
| | LGDKWDKEEDVQVLAIEPGKN | | |
| | PKHVQTKPGLFKTLTGEIGAVT | | |
| | LDFKPGTSGSPIINRKGKVIGLY | | |
| | GNGWTKSGDYVSAITQAERIG | | |
| | EPDYEVDEDIFRKKRLTIMDLH | | |
| | PGAGKTKRILPSIVREALKRRL | | |
| | RTLILAPTRWAAEMEEALRGL | | |
| | PIRYQTPAVKSEHTGREIVDLM | | |
| | CHATFTTRLLSSTRVPNYNLIV | | |
| | MDEAHFTDPSSVAARGYISTRV | | |
| | EMGEAAAIFMTATPPGTTDPFP | | |
| | QSNSPIEDIEREIPERSWNTGFD | | |
| | WITDYQGKTVWFVPSIKAGND | | |
| | IANCLRKSGKKVIQLSRKTFDT | | |
| | EYPKTKLTDWDFWTTDISEM | | |
| | GANFRAGRVIDPRRCLKPVILP | | |
| | DGPERVILAGPIPVTPASAAQR | | |
| | RGRIGRNPAQEDDQYVFSGDP | | |
| | LKNDEDHAHWTEAKMLLDNI | | |
| | YTPEGIIPTLFGPEREKTQAIDG | | |
| | EFRLRGEQRKTFVELMRRGDL | | |
| | PVWLSYKVASAGISYKDREWC | | |
| | FTGERNNQILEENMEVEIWTRE | | |
| | GEKKKLRPKWLDARVYADPM | | |
| | ALKDFKEFASGRK | | |
| | (SEQ ID NO: 29) | | |

Exemplary proteases which can be used in fusion proteins of the present disclosure include hepatitis C virus proteases (e.g., NS3 and NS2-3); signal peptidase, proprotein convertases of the subtilisin/kexin family (furin, PCI, PC2, PC4, PACE4, PC5, PC), proprotein convertases cleaving at hydrophobic residues (e.g., Leu, Phe, Val, or Met), proprotein convertases cleaving at small amino acid residues such as Ala or Thr, proopiomelanocortin converting enzyme (PCE); chromaffin granule aspartic protease (CGAP); prohormone thiol protease; carboxypeptidases (e.g., carboxypeptidase E/H, carboxypeptidase D and carboxypeptidase Z); aminopeptidases (e.g., arginine aminopeptidase, lysine aminopeptidase, aminopeptidase B), prolyl endopeptidase; aminopeptidase N; insulin degrading enzyme, calpain; high molecular weight protease; and, caspases 1, 2, 3, 4, 5, 6, 7, 8, and 9. Other proteases include, but are not limited to, aminopeptidase N; puromycin sensitive aminopeptidase; angiotensin converting enzyme; pyroglutamyl peptidase II; dipeptidyl peptidase IV; N-arginine dibasic convertase; endopeptidase 24.15; endopeptidase 24.16; amyloid precursor protein secretases alpha, beta and gamma; angiotensin converting enzyme secretase; TGF alpha secretase; T F alpha secretase; FAS ligand secretase, TNF receptor-I and -II secretases; CD30 secretase; KL1 and KL2 secretases; IL6 receptor secretase; CD43, CD44 secretase; CD 16-1 and CD 16-11 secretases; L-selectin secretase; Folate receptor secretase; MMP 1, 2, 3, 7, 8, 9, 10, 11, 12, 13, 14, and 15; urokinase plasminogen activator; tissue plasminogen activator; plasmin; thrombin; BMP-1 (procollagen C-peptidase); ADAM 1, 2, 3, 4, 5, 6, 7, 8, 9, 10, and 11; and, granzymes A, B, C, D, E, F. G, and H. The protease chosen for use in the fusion protein is preferably highly selective for the cleavage site in the cleavable linker. Additionally, protease activity is preferably inhibitable with inhibitors that are cell-permeable and not toxic to the cell or subject under study. For a discussion of proteases, see, e.g., V. Y. H. Hook, Proteolytic and cellular mechanisms in prohormone and proprotein processing, RG Landes Company. Austin, Tex., USA (1998); N. M. Hooper et al., Biochem. J. 321: 265-279 (1997); Z. Werb, Cell 91: 439-442 (1997); T. G. Wolfsberg et al., J. Cell Biol. 131: 275-278 (1995): K. Murakami and J. D. Etlinger, Biochem. Biophys. Res. Comm. 146: 1249-1259 (1987): T. Berg et al., Biochem. J. 307: 313-326 (1995): M. J. Smyth and J. A. Trapani, Immunology Today 16: 202-206 (1995); R. V. Talanian et al., J. Biol. Chem. 272: 9677-9682 (1997), and N A. Thomberry et al, J Biol Chem. 272: 17907-17911 (1997), the disclosures of which are incorporated herein.

In certain embodiments, the protease used in the fusion protein is derived from hepatitis C virus (HCV). In some embodiments, the protease is an HCV nonstructural protein 3 (NS3) protease. NS3 contains an N-terminal serine protease domain and a C-terminal helicase domain. The protease domain of NS3 forms a heterodimer with the HCV nonstructural protein 4A (NS4A co-factor), which activates proteolytic activity. An NS3 protease may comprise the entire NS3 protein or a proteolytically active fragment thereof and may further comprise an activating NS4A co-factor region. Advantages of using an NS3 protease include that it is highly selective and can be well-inhibited by a number of non-toxic, cell-permeable drugs, which are currently clinically available. NS3 protease inhibitors that can be used in the practice of the present disclosure include, but are not limited to, simeprevir, danoprevir, asunaprevir, ciluprevir, boceprevir, sovaprevir, paritaprevir, telaprevir, grazoprevir, glecaprevir, and voxiloprevir.

When an NS3 protease is used in a fusion protein, the cleavable linker of the fusion protein may comprise an NS3 protease cleavage site (e.g., a cognate cleavage site). Exemplary NS3 protease cleavage sites, which can be used in the cleavable linker, include the four junctions between nonstructural (NS) proteins of the HCV polyprotein normally cleaved by the NS3 protease during HCV infection, including the NS3/NS4A, NS4A/NS4B, NS4B/NS5A, and NS5A/NS5B junction cleavage sites. For a description of NS3 protease and representative sequences of its cleavage sites for various strains of HCV, see, e.g., Hepatitis C Viruses: Genomes and Molecular Biology (S. L. Tan ed., Taylor & Francis, 2006), Chapter 6, pp. 163-206; herein incorporated by reference in its entirety.

NS3 nucleic acid and protein sequences may be derived from HCV, including any isolate of HCV having any genotype (e.g., seven genotypes 1-7) or subtype. A number of NS3 nucleic acid and protein sequences are known. A representative NS3 sequence is presented in Table 1. Additional representative sequences are listed in the National Center for Biotechnology Information (NCBI) database See, for example, NCBI entries: Accession Nos. YP_001491553, YP_001469631, YP_001469632, NP 803144, NP 671491, YP_001469634, YP_001469630, YP_001469633, ADA68311, ADA68307, AFP99000, AFP98987, ADA68322, AFP99033, ADA68330, AFP99056, AFP99041, CBF60982, CBF60817, AHH29575, AIZ00747, AIZ00744, AB136969, ABN05226, KF516075, KF516074, KF516056, AB826684. AB826683, JX171009, JX171008, JX171000, EU847455, EF154714, GU085487, JX171065, JX171063, all of which sequences (as entered by the date of filing of this application) are herein incorporated by reference. Any of these sequences or a variant thereof comprising a sequence having at least about 80-100° % sequence identity thereto, including any percent identity within this range, such as 81, 82, 83, 84, 85, 86, 87, 88, 89, 90, 91, 92, 93, 94, 95, 96, 97, 98, or 99% sequence identity thereto, can be used to construct a fusion protein or a recombinant polynucleotide encoding such a fusion protein, as described herein.

NS4A nucleic acid and protein sequences may be derived from HCV, including any isolate of HCV having any genotype (e.g., seven genotypes 1-7) or subtype. A number of NS4 A nucleic acid and protein sequences are known Representative sequences are listed in the National Center for Biotechnology Information (NCBI) database. See, for example, NCBI entries: Accession Nos. NP_751925, YP_001491554, GU945462, HQ822054, FJ932208, FJ932207, FJ932205, and FJ932199; all of which sequences (as entered by the date of filing of this application) are herein incorporated by reference Any of these sequences or a variant thereof comprising a sequence having at least about 80-100%) sequence identity thereto, including any percent identity within this range, such as 81, 82, 83, 84, 85, 86, 87, 88, 89, 90, 91, 92, 93, 94, 95, 96, 97, 98, or 99% sequence identity thereto, can be used to construct a fusion protein or a recombinant polynucleotide encoding such a fusion protein, as described herein.

HCV polyprotein nucleic acid and protein sequences may be derived from HCV, including any isolate of HCV having any genotype (e.g., seven genotypes 1-7) or subtype. A number of HCV polyprotein nucleic acid and protein sequences are known. Representative HCV polyprotein sequences are listed in the National Center for Biotechnology Information (NCBI) database See, for example, NCI entries. Accession Nos YP_001469631, P_671491, YP_001469633, YP_001469630, YP_001469634. YP_001469632, NC 009824. NC 004102, NC_009825, NC_009827, NC_009823, NC_009826, and EF 108306; all of which sequences (as entered by the date of filing of this application) are herein incorporated by reference. Any of these sequences or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto, can be used to construct a fusion protein or a recombinant polynucleotide encoding such a fusion protein, as described herein.

In some embodiments, the NS3 protease is derived from HCV 1a. In some embodiments, the HCV 1a polyprotein has the following amino acid sequence (SEQ ID NO: 1):

```
                10 20 30 40 50
MSTNPKPQKK NKRNTNRRPQ DVKFPGGGQI VGGVYLLPRR GPRLGVRATR
                60 70 80 90 100
KTSERSQPRG RRQPIPKARR PEGRTWAQPG YPWPLYGNEG CGWAGWLLSP
              110 120 130 140 150
RGSRPSWGPT DPRRRSRNLG KVIDTLTCGF ADLMGYIPLV GAPLGGAARA
              160 170 180 190 200
LAHGVRVLED GVNYATGNLP GCSFSIFLLA LLSCLTVPAS AYQVRNSTGL
              210 220 230 240 250
YHVTNDCPNS SIVYKAADAI LHTPGCVPCV REGNASRCWV AMTPTVATRD
              260 270 280 290 300
GKLPATQLRR HIDLLVGSAT LCSALYVGDL CGSVFLVGQL FTFSPRRHWT
              310 320 330 340 350
TQGCNCSIYP GHITGHRMAW DMMMNWSPTT ALVMAQLLRI PQAILDMIAG
              360 370 380 390 400
AHWGVLAGIA YFSMVGNWAK VLVVLLLFAG VDAETHVTGG SAGHTVSGFV
              410 420 430 440 450
SLLAPGAKQN VQLINTNGSW HLNSTALNCN DSLNTGWLAG LFYHHKENSS
              460 470 480 490 500
GCPERLASCR PLTDFDQGWG PISYANGSGP DQRPYCWHYP PKPCGIVPAK
              510 520 530 540 550
SVCGPVYCFT PSPVVVGTTD RSGAPTYSWG ENDTDVFVLN NTRPPLGNWF
              560 570 580 590 600
GCTWMNSTGF TKVCGAPPCV IGGAGNNTLH CPTDCFRKHP DATYSRCGSG
              610 620 630 640 650
PWITPRCLVD YPYRLWHYPC TINYTIFKIR MYVGGVEHRL EAACMWTRGE
              660 670 680 690 700
RCDLEDRDRS ELSPLLLTTT QWQVLPCSFT TLPALSTGLI HLHQNIVDVQ
              710 720 730 740 750
YLYGVGSSIA SWAIKWEYVV LLFLLLADAR VCSCLWMMLL ISQAEAALEN
              760 770 780 790 800
LVILNAASLA GTHGLVSFLV FFCFAWYLKG KWVPGAVYTF YGMWPILLLL
              810 820 830 840 850
LALPQRAYAL DTEVAASCGG VVLVGLMALT LSPYYKEYIS WCLWWLQYFL
              860 870 880 890 900
TRVEAQLHVW IPPLNVRGGR DAVILLMCAV HPTLVEDITK LLLAVFGPLW
              910 920 930 940 950
ILQASLLKVP YFVRVQGLLR FCALARKMIG GHYVQMVIIK LGALTGTYVY
              960 970 980 990 1000
NHLTPLRDWA HNGLRDLAVA VEPVVFSQME TKLITWGADT AACGDIINGL
            1010 1020 1030 1040 1050
PVSARRGREI LLGPADGMVS KGWRLLAPIT AYAQQTRGLL GCIITSLTGR
            1060 1070 1080 1090 1100
DKNQVEGEVQ IVSTAAQTFL ATCINGVCWT VYHGAGTRTI ASPKGPVIQM
            1110 1120 1130 1140 1150
YTYVDQDLVG WPAPQGSRSL TPCTCGSSDL YLVTRHADVI PVRRRGDSRG
            1160 1170 1180 1190 1200
SLLSPRPISY LKGSSGGPLL CPAGHAVGIF RAAVCTRGVA KAVDFIPVEN
            1210 1220 1230 1240 1250
LETTMRSPVF TDNSSPPVVP QSFQVAHLHA PTGSGKSTKV PAAYAAQGYK
            1260 1270 1280 1290 1300
VLVLNPSVAA TLGFGAYMSK AEGIDPNIRT GVRTITTGSP ITYSTYGKFL
            1310 1320 1330 1340 1350
ADGGCSGGAY DIIICDECHS TDATSILGIG TVLDQAETAG ARLVVLATAT
            1360 1370 1380 1390 1400
PPGSVTVPHP NIEEVALSTT GEIPFYGKAI PLEVIKGGRH LIFCHSKKKC
            1410 1420 1430 1440 1450
DELAAKLVAL GINAVAYYRG LDVSVIPTSG DVVVVATDAL MTGYTGDFDS
            1460 1470 1480 1490 1500
VIDCNTCVTQ TVDFSLDPTF TIETITLPQD AVSRTQRRGR TGRGKPGIYR
            1510 1520 1530 1540 1550
FVAPGERPSG MFDSSVLCEC YDAGCAWYEL TPAETTVRLR AYMNTPGLPV
            1560 1570 1580 1590 1600
CQDHLEFWEG VYTGLTHIDA HFLSQTKQSG ENLPYLVAYQ ATVCARAQAP
            1610 1620 1630 1640 1650
PPSWDQMWKC LIRLKPTLHG PTPLLYRLGA VQNEITLTHP VTKYIMTCMS
            1660 1670 1680 1690 1700
ADLEVVTSTW VLVGGVLAAL AAYCLSTGCV VIVGRVVLSG KPAIIPDREV
            1710 1720 1730 1740 1750
LYREFDEMEE CSQHLPYIEQ GMMLAEQFKQ KALGLLQTAS RQAEVIAPAV
            1760 1770 1780 1790 1800
QTMWQKLETF WAKHMWMFIS GIQYLAGLST LPGNPAIASL MAFTAAVTSP
            1810 1820 1830 1840 1850
LTTSQTLLFN ILGGWVAAQL AAPGAATAFV GAGLAGAAIG SVGLGKVLID
            1860 1870 1880 1890 1900
ILAGYGAGVA GALVAFKIMS GEVPSTEDLV NLLPAILSPG ALVVGVVCAA
            1910 1920 1930 1940 1950
ILRRHVGPGE GAVQWMNREI AFASRGNHVS PTHYVPESDA AARVTAILSS
            1960 1970 1980 1990 2000
LTVTQLLRRL HQWISSECTT PCSGSWLRDI WDWICEVLSD FKTWLKAELM
            2010 2020 2030 2040 2050
PQLPGIPFVS CQRGYKGVWR VDGIMHTRCE CGAEITGHVK NGTMRIVGPR
            2060 2070 2080 2090 2100
TCRNMWSGTF PINAYTTGPC TPLPAPNYTF ALWRVSAEEY VEIRQVGDFH
            2110 2120 2130 2140 2150
YVTGMTTDNL KCPCQVPSPE FFTELDGVRL HRFAPPCKPL LREEVSFRVG
            2160 2170 2180 2190 2200
LHEYPVGSQL PCEPEPDVAV LTSMLTDPSH ITAEAAGRRL ARGSPPSVAS
            2210 2220 2230 2240 2250
SSASQLSAPS LKATCTANHD SPDAELIEAN LLWRQEMGGN ITRVESENRV
            2260 2270 2280 2290 2300
VILDSFDPLV AEEDEREISV PAEILRKERR FAQALPVWAR PDYNPPLVET
            2310 2320 2330 2340 2350
WKKPDYEPPV VHGCPLPPPK SPPVPPPRKK RTVVLTESTL STALAELATR
            2360 2370 2380 2390 2400
SFGSSSTSGI TGDNTTTSSE PAPSGCPPDS DAESYSSMPP LEGEPGDPDL
            2410 2420 2430 2440 2450
SDGSWSTVSS EANAEDVVCC SMSYSWTGAL VTPCAAEEQK LPINALSNSL
            2460 2470 2480 2490 2500
LRHHNLVYST TSRSACQRQK KVTFDRLQVL DSHYQDVLKE VKAAASKVKA
            2510 2520 2530 2540 2550
NLLSVEEACS LTPPHSAKSK FGYGAKDVRC HARKAVTHIN SVWKDLLEDN
            2560 2570 2580 2590 2600
VTPIDTTIMA KNEVECVQPE KGGRKPARII VFPDLGVRVC EKMALYDVVT
            2610 2620 2630 2640 2650
KLPLAVMGSS YGFQYSPGQR VEFLVQAWKS KKTPMGESYD TRCFDSTVTE
            2660 2670 2680 2690 2700
SDIRTEEAIY QCCDLDPQAR VAIKSLTERL YVGGPLTNSR GENCGYRRCR
            2710 2720 2730 2740 2750
ASGVLTTSCG NTLTCYIKAR AACRAAGLQD CTMLVCGDDL VVICESAGVQ
            2760 2770 2780 2790 2800
EDAASLRAFT EAMTRYSAPP GDPPQPEYDL ELITSCSSNV SVAHDGAGKR
            2810 2820 2830 2840 2850
VYYLTRDPTT PLARAAWETA RHTPVNSWLG NIIMFAPTLW ARMILMTHFF
            2860 2870 2880 2890 2900
SVLIARDQLE QALDCEIYGA CYSIEPLDLP PlIQRLHGLS AFSLHSYSPG
            2910 2920 2930 2940 2950
EINRVAACLR KLGVPPLRAW RHRARSVRAR LLARGGRAAI CGKYLFNWAV
            2960 2970 2980 2990 3000
RTKLKLTPIA AAGQLDLSGW FTAGYSGGDI YHSVSHARPR WIWFCLLLLA
            3010
AGVGIYLLPN R
```

In some embodiments, a fusion proteins of the present disclosure comprise a variant NS3 protease derived from the HCV 1a polyprotein having the amino acid sequence of SEQ ID NO. 1 In some embodiments, the variant protease comprises one or more mutations, such as amino acid substitutions, that decrease immunogenicity. In some embodiments, the variant protease comprises two or more mutations, three or more mutations, four or more mutations, five or more mutations, six or more mutations, seven or more mutations, eight or more mutations, nine or more mutations, 10 or more mutations, 11 or more mutations, 12 or more mutations, 13 or more mutations, 14 or more mutations, 15 or more mutations, 16 or more mutations, 17 or more mutations, 18 or more mutations, 19 or more mutations, or 20 or more mutations. In some embodiments, the variant protease comprises 1 mutation, 2 mutations, 3 mutations, 4 mutations, 5 mutations, 6 mutations, 7 mutations, 8 mutations, 9 mutations, 10 mutations, 1 mutations, 12 mutations, 13 mutations, 14 mutations, 15 mutations, 16 mutations, 17 mutations, 18 mutations, 19 mutations, or 20 mutations. In some embodiments the one or more mutations are amino acid substitutions.

The variant protease may include one or more mutations within an immunodominant epitope that results in a reduction in immunogenicity of the protease and/or within an epitope that that results in modulation of the catalytic activity of the protease (see e.g., Söerholm J, et al. Gut. 2006 February; 55(2):266-74; Soumana D et al. ACS Chem Biol. 2014 Nov. 21; 9(11):2485-90; and Wertheimer A M et al. Hepatology. 2003 March; 37(3):577-89). For example, the one or more mutations may be within a region corresponding to positions 1038 to 1047 of SEQ ID NO: 1, positions 1057 to 1081 of SEQ ID NO: 1, positions 1073 to 1081 of SEQ ID NO: 1, positions 1073 to 1082 of SEQ ID NO 1, positions 1127 to 1141 of SEQ ID NO. 1, positions 1131 to 1138 of SEQ ID NO: 1, positions 1169 to 1177 of SEQ ID NO: 1, and/or positions 1192 to 1206 of SEQ ID NO 1. In some embodiments, the one or more mutations may be within a region selected from GLLGCIITSL (SEQ ID NO: 30), GEVQIVSTAAQTFLATCINGVCWTVY (SEQ ID NO: 31), GEVQIVSTAAQTFLA (SEQ ID NO. 32), QTFLATCINGVCWTV (SEQ ID NO: 33), CINGVCWTVY (SEQ ID NO: 34), SSDLYLVTRHADVIP (SEQ ID NO: 35), YLVTRHAD (SEQ ID NO: 36), LLCPAGHAV (SEQ ID NO: 37), AVDFIPVEGLETTMR (SEQ ID NO: 38), KIDTKYIMTCMSADL (SEQ ID NO. 39), and any combination thereof.

In some embodiments, the one or more mutations are one or more amino acid substitutions selected from a position corresponding to position 1062 of SEQ ID NO. 1, a position corresponding to position 1069 of SEQ ID NO: 1, a position corresponding to position 1070 of SEQ ID NO 1, a position corresponding to position 1071 of SEQ ID NO: 1, a position corresponding to position 1072 of SEQ ID NO: 1, a position corresponding to position 1074 of SEQ ID NO. 1, a position corresponding to position 1075 of SEQ ID NO 1, a position corresponding to position 1077 of SEQ ID NO: 1, a position corresponding to position 1078 of SEQ ID NO 1, a position corresponding to position 1079 of SEQ ID NO. 1, a position corresponding to position 1080 of SEQ ID NO: 1, a position corresponding to position 1031 of SEQ ID NO. 1, a position corresponding to position 1074 of SEQ ID NO 1, a position corresponding to position 1132 of SEQ ID NO: 1, a position corresponding to position 1133 of SEQ ID NO 1, a position corresponding to position 1195 of SEQ ID NO. 1, a position corresponding to position 1196 of SEQ ID NO: 1, a position corresponding to position 1201 of SEQ ID NO: 1, a position corresponding to position 1202 of SEQ ID NO: 1, and any combination thereof.

In some embodiments, the one or more mutations are one or more amino acid substitutions selected from an Ile to Leu substitution at a position corresponding to position 1074 of SEQ ID NO: 1, an Ile to Met substitution at a position corresponding to position 1074 of SEQ ID NO: 1, an Asn to Ala substitution at a position corresponding to position 1075 of SEQ ID NO: 1, a Val to Ala substitution at a position corresponding to position 1077 of SEQ ID NO: 1, a Cys to Phe substitution at a position corresponding to position 1078 of SEQ ID NO: 1, a Trp to Ala substitution at a position corresponding to position 1079 of SEQ ID NO: 1, a Thr to Ala substitution at a position corresponding to position 1080 of SEQ ID NO: 1, a Val to Ala substitution at a position corresponding to position 1081 of SEQ ID NO: 1, a Val to Asn substitution at a position corresponding to position 1081 of SEQ ID NO: 1, and any combination thereof. In some embodiments, the one or more mutations are one or more amino acid substitutions selected from a Thr to Ala substitution at a position corresponding to position 1080 of SEQ ID NO. 1, a Val to Ala substitution at a position corresponding to position 1077 of SEQ ID NO: 1, a Val to Ala substitution at a position corresponding to position 1081 of SEQ ID NO: 1, and any combination thereof. In some embodiments, the one or more mutations comprise a Thr to Ala substitution at a position corresponding to position 1080 of SEQ ID NO: 1. In some embodiments, the one or more mutations comprise a Thr to Ala substitution at a position corresponding to position 1080 of SEQ ID NO 1 and a Val to Ala substitution at a position corresponding to position 1077 of SEQ ID NO: 1. In some embodiments, the one or more mutations comprise a Thr to Ala substitution at a position corresponding to position 1080 of SEQ ID NO: 1 and a Val to Ala substitution at a position corresponding to position 1081 of SEQ ID NO: 1.

In some embodiments, the variant protease may comprise one or more additional mutations, such as amino acid substitutions, that tune or otherwise modulate the enzymatic activity of the protease. In some embodiments, the variant protease comprises two or more additional mutations, three or more additional mutations, four or more additional mutations, five or more additional mutations, six or more additional mutations, seven or more additional mutations, eight or more additional mutations, nine or more additional mutations, or 10 or more additional mutations. In some embodiments, the variant protease comprises 1 additional mutation, 2 additional mutations, 3 additional mutations, 4 additional mutations, 5 additional mutations, 6 additional mutations, 7 additional mutations, 8 additional mutations, 9 additional mutations, or 10 additional mutations. In some embodiments the one or more additional mutations are amino acid substitutions. In some embodiment, the one or more additional mutations are amino acid substitutions at one more positions corresponding to position 1074 of SEQ ID NO: 1, position 1078 of SEQ ID NO: 1 and/or position 1079 of SEQ ID NO: 1. In some embodiment, the one or more additional mutations decrease the enzymatic activity of the protease. In some embodiments, the one or more additional mutations that decrease the enzymatic activity of the protease are one or more additional amino acid substitutions selected from an lie to Ala substitution at a position corresponding to position 1074 of SEQ ID NO 1, a Trp to Ala substitution at a position corresponding to position 1079 of SEQ ID NO: 1, and any combination thereof in some embodiment, the one or more additional mutations increase the enzymatic activity of the protease. In some embodiments, the one or more additional mutations that increase the enzymatic activity of the protease are one or more additional amino acid substitutions that include a Cys to Ala substitution at a position corresponding to position 1078 of SEQ ID NO: 1.

In some embodiments, a fusion protein of the present disclosure comprise a variant NS3 protease derived from the HCV NS3 protease having an amino acid sequence of:

(SEQ ID NO. 2)
APITAYAQQTRGLLGCIITSLTGRDKNQVEGEVQIVSTATQTFLATC
INGVCWAVYHGAGTRTIASPKGPVIQMYTNVDQDLVGWPAPQGSRSL
TPCTCGSSDLYLVTRHADVIPVRRRGDSRGSLLLSPRPISYLKGSSG
GPLLCPAGHAVGLFRAAVCTRGVAKAVDFIPVENLETTMRSPVFTD.

In some embodiments, the fusion protein further comprises an HCV NS4A co-factor. In some embodiments, the NS4A co-factor has the amino acid sequence of

(SEQ ID NO: 3)
TWVLVGGVLAALAAYCLSTGCVVIVGRWLSGKPAEPDREVLY.

### Cognate Protease Cleavage Sites

Certain aspects of the present disclosure relate to a fusion protein comprising a variant protease and a cognate cleavage site recognized by the protease. When a protease is selected, its cognate cleavage site and protease inhibitors known in the art to bind and inhibit the protease may be used in a combination. Any suitable protease, cognate cleavage site and cognate protease inhibitor may be used. Exemplary combinations or proteases, cognate cleavage sites and cognate protease inhibitors are provided below in Table 1.

When an NS3 protease is used, the cognate cleavage site comprises an NS3 protease cleavage site Exemplary NS3 protease cleavage sites include the four junctions between nonstructural (NS) proteins of the HCV polyprotein normally cleaved by the NS3 protease during HCV infection, including the NS3/NS4A, NS4A/NS4B, NS4B/NS5A, and NS5A/NS5B junction cleavage sites. For a description of NS3 protease and representative sequences of its cleavage sites for various strains of HCV, see, e.g., Hepatitis C Viruses Genomes and Molecular Biology (S. L. Tan ed., Taylor & Francis, 2006), Chapter 6, pp. 163-206; herein incorporated by reference in its entirety. For example, the sequences of HCV NS3/4A protease cleavage sites, HCV NS4A/4B protease cleavage sites (SEQ ID NO. 9, 44); HCV NS4B/5A protease cleavage sites; and HCV NS5A/5B protease cleavage sites (SEQ ID NO: 11, 45) are provided in Table 1.

In some embodiments, cognate cleavage sites for NS3 protease include those listed in Table 1. In some embodiments, a cognate cleavage site for an NS3 protease, such as a variant NS3 protease of the present disclosure, is selected from CMSADLEVVTSTWVLVGGVL (SEQ ID NO: 4), YQEFDEMEECSQHLPYIEQG (SEQ ID NO: 5), WISSECTTPCSGSWLRDIWD (SEQ ID NO: 6), and GADTEDVVCCSMSYSWTGAL (SEQ ID NO: 7). In some embodiment, a cognate cleavage site for an NS3 protease, such as a variant NS3 protease of the present disclosure, is selected from ADLEVVTSTWL (SEQ ID NO: 8), DEMEECSQHL (SEQ ID NO: 9), ECTTPCSGSWL (SEQ ID NO: 10), and EDVVPCSMG (SEQ ID NO: 11). In some embodiments, the cognate cleavage site comprises one or more mutations, such as one or more amino acid substitutions. In some embodiments, mutations in the cognate cleavage site can tune, or otherwise modulate, the enzymatic activity and/or catalytic rate of the protease. For example, in some embodiments, the one or more mutations can increase the enzymatic activity and/or catalytic rate of the protease. Alternatively, in some embodiments, the one or more mutations can decrease the enzymatic activity and/or catalytic rate of the protease.

### Degrons

Certain aspects of the present disclosure relate to a fusion protein comprising a polypeptide of interest, a protease, a cognate protease cleavage site, and that further comprises a degron or a self-excising degron.

Degrons of the present disclosure may comprise a sequence of amino acids, which provides a degradation signal that directs a polypeptide for cellular degradation. The degron may promote degradation of an attached polypeptide through either the proteasome or autophagy-lysosome pathways. In a fusion protein of the present disclosure, the degron must be operably linked to the polypeptide of interest, but need not be contiguous with it as long as the degron still functions to direct degradation of the polypeptide of interest. Preferably, the degron induces rapid degradation of the polypeptide of interest. For a discussion of degrons and their function in protein degradation, see, e.g., Kanemaki et al (2013) Pflugers Arch. 465(3) 419-425, Erales et al. (2014) Biochim Biophys Acta 1843(1):216-221, Schrader et al. (2009) Nat. Chem. Biol. 5(11) 815-822, Ravid et al. (2008) Nat Rev. Mol. Cell. Biol. 9(9) 679-690, Tasaki et al. (2007) Trends Biochem Sci. 32(11):520-528, Meinnel et al. (2006) Biol. Chem. 387(7):839-851, Kim et al. (2013) Autophagy 9(7): 1100-1103, Varshavsky (2012) Methods Mol. Biol. 832: 1-11, and Fayadat et al. (2003) Mol Biol Cell. 14(3): 1268-1278, herein incorporated by reference.

Degrons with degradation sequences known in the art may be used for various embodiments of the present disclosure. In some embodiments, a degron of the present disclosure may be derived from a degron identified from an organism, or a modification thereof. Such a degron includes, but not limited to, an HCV NS4 degron, a PEST (Two copies of residues 277-307 of IκBα(human) (SEQ ID NO: 46), a GRR (residues 352-408 of p105 (human) (SEQ ID NO: 47), a DRR (residue 210-295 of Cdc34 (yeast) (SEQ ID NO: 48), an SNS (tandem repeat of SP2 and NB (SP2-NB-SP2) (Influenza A and B) (SEQ ID NO 49), an RPB (four copies of residues 1688-1702 of RPB1 (yeast) (SEQ ID NO: 50), an SPmix (tandem repeat of SP1 and SP2 (SP2-SP1-SP2-SP1-SP2) (Influenza A virus M2 protein) (SEQ ID NO 51), an NS2 (three copies of residue 79-93 of Influenza A virus NS protein) (SEQ ID NO: 52), an ODC (residue 106-142 of ornithine decarboxylase) (SEQ ID NO: 53), a Nek2A (human), an mODC (amino acids 422-461 (moue), an mODC_DA (amino acids 422-461 of mODC (D433A, D434A point mutations (mouse)) (SEQ ID NO: 54), an APC/C degrons (e.g., D box, KEN box and ABBA motif), a COP1 E3 ligase binding degron motif, a CRL4-Cdt2 binding PIP degron, an actinfilin-binding degron, a KEAP1 binding degron, a KLHL2 and KLHL3 binding degron, an MDM2 binding motif, an N-degron (e.g., Nbox, or UBRbox), a hydroxyproline modification in hypoxia signaling, a phytohormone-dependent SCF-LRR-binding degron, an SCF ubiquitin ligase binding phosphodegron, a phytohormone-dependent SCF-LRR-binding degron, a DSGxxS (SEQ ID NO: 55) phospho-dependent degron, a siah binding Motif, an SPOP SBC docking motif, and a PCNA binding PIP box.

In some embodiments the degron comprises portions of the HCV nonstructural proteins NS3 and NS4A. In one embodiment, the degron comprises the amino acid sequence of PITKIDTKYIMTCMSADLEVVTSTWVLVGGVLAALAAYCLST (SEQ ID NO: 40) or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto, wherein the degron is capable of promoting degradation of a polypeptide. It is to be understood that degrons comprising the residues corresponding to the reference sequence of SEQ ID NO. 40 in I-iCV nonstructural proteins NS3 and NS4A obtained from other strains of HCV are also intended to be encompassed by the present disclosure.

In the fusion protein, the degron may be linked to the N-terminus or the C-terminus of the polypeptide of interest. For example, the fusion protein can be represented by the formula NH₂-P-D-L-X-COOH or NH₂-X-L-P-D-COOH, wherein: P is an amino acid sequence of a protease; D is an amino acid sequence of a degron; L is an amino acid sequence of a linker comprising a cleavage site for the protease; and X is an amino acid sequence of a selected polypeptide of interest. The cleavable linker between the polypeptide of interest and the degron is designed for selective cleavage by the particular protease included in the fusion protein. The cleavage site of the linker includes the specific amino acid sequence recognized by the protease during proteolytic cleavage and typically includes the surrounding one to six amino acids on either side of the scissile bond, which bind to the active site of the protease and are needed for recognition as a substrate. The cleavable linker may contain any protease recognition motif known in the art and is typically cleavable under physiological conditions.

The polypeptides included in the fusion construct may be connected directly to each other by peptide bonds or may be separated by intervening amino acid sequences. The fusion polypeptides may also contain sequences exogenous to the protease or the selected protein of interest. For example, the fusion protein may include targeting or localization sequences, tag sequences, or sequences of fluorescent or bioluminescent proteins.

In certain embodiments, tag sequences are located at the N-terminus or C-terminus of the fusion protein. Exemplary tags that can be used in the practice of the present disclosure include a His-tag, a Strep-tag, a TAP-tag, an S-tag, an SBP-tag, an Arg-tag, a calmodulin-binding peptide tag, a cellulose-binding domain tag, a DsbA tag, a c-myc tag, a glutathione S-transferase tag, a FLAG tag, a HAT-tag, a maltose-binding protein tag, a NusA tag, and a thioredoxin tag.

In certain embodiments, the fusion protein comprises a targeting sequence Exemplary targeting sequences that can be used in the practice of the present disclosure include a secretory protein signal sequence, a membrane protein signal sequence, a nuclear localization sequence, a nucleolar localization signal sequence, an endoplasmic reticulum localization sequence, a peroxisome localization sequence, a mitochondrial localization sequence, and a protein-protein interaction motif sequence. Examples of targeting sequences include those targeting the nucleus (e.g., KKKRK, SEQ ID NO: 41), mitochondrion (e.g., MLRT S SLFTRRVQP SLFRNILRLQ ST, SEQ ID NO. 42), endoplasmic reticulum (e.g., KDEL, SEQ ID NO. 43), peroxisome (e.g., SKL), synapses (e.g., S/TDV or fusion to GAP 43, kinesin or tau), plasma membrane (e.g., CaaX) where “a” is an aliphatic amino acid, CC, CXC, CCXX at C-terminus), or protein-protein interaction motifs (e.g., SH2, SH3, PDZ, WW, RGD, Src homology domain, DNA-binding domain, SLiMs).

In certain embodiments, the fusion protein comprises a detectable label. The detectable label may comprise any molecule capable of detection. Detectable labels that may be used in the practice of the present disclosure include, but are not limited to, radioactive isotopes, stable (non-radioactive) heavy isotopes, fluorescers, chemiluminescers, enzymes, enzyme substrates, enzyme cofactors, enzyme inhibitors, chromophores, dyes, metal ions, metal sols, ligands (e.g., biotin or haptens) and the like. Particular examples of labels that may be used with the present disclosure include, but are 3 125 35 14 32 not limited to radiolabels (e.g., H, I, S, C, or P), stable (non-radioactive) heavy isotopes (e.g., ¹³C or ¹⁵N), phycoerythrin, Alexa dyes, fluorescein, 7-nitrobenzo-2-oxa-1,3-diazole (NBD), YPet, CyPet, Cascade blue, allophycocyanin, Cy3, Cy5, Cy7, rhodamine, dansyl, umbelliferone, Texas red, luminol, acradimum esters, biotin or other streptavidin-binding proteins, magnetic beads, electron dense reagents, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), Dronpa, Padron, mApple, mCherry, rsCherry, rsCherryRev, firefly luciferase, Renilla luciferase, NADPH, beta-galactosidase, horseradish peroxidase, glucose oxidase, alkaline phosphatase, chloramphenical acetyl transferase, and urease Enzyme tags are used with their cognate substrate. The terms also include color-coded microspheres of known fluorescent light intensities (see e.g., microspheres with xMAP technology produced by Luminex (Austin, Tex.); microspheres containing quantum dot nanocrystals, for example, containing different ratios and combinations of quantum dot colors (e.g., Qdot nanocrystals produced by Life Technologies (Carlsbad, Calif.); glass coated metal nanoparticles (see e.g., SERS nanotags produced by Nanoplex Technologies. Inc. (Mountain View, Calif.); barcode materials (see e.g., sub-micron sized striped metallic rods such as Nanobarcodes produced by Nanoplex Technologies, Inc.), encoded microparticles with colored bar codes (see e.g., CellCard produced by Vitra Bioscience, vitrabio.com), and glass microparticles with digital holographic code images (see e.g, CyVera microbeads produced by Illumina (San Diego, Calif.). As with many of the standard procedures associated with the practice of the present disclosure, skilled artisans will be aware of additional labels that can be used.

### Polypeptides of Interest

In one aspect, the present disclosure provides a fusion protein comprising a polypeptide of interest. The polypeptide of interest selected for inclusion in the fusion protein may be from a membrane protein, a receptor, a hormone, a transport protein, a transcription factor, a cytoskeletal protein, an extracellular matrix protein, a signal-transduction protein, an enzyme, or any other protein of interest. The polypeptide of interest may comprise an entire protein, or a biologically active domain (e.g., a catalytic domain, a ligand binding domain, or a protein-protein interaction domain), or a polypeptide fragment of a selected protein. In some embodiments, the polypeptide of interest comprises one or more functional and/or structural domains. In some embodiments, the polypeptide of interest comprises multiple functional and/or structural domains.

In some embodiments, the polypeptide of interest is a therapeutic protein. Examples of suitable therapeutic proteins include, but are not limited to, receptors, antibodies, Fc fusion proteins, anticoagulants, blood factors, bone morphogenetic proteins, engineered protein scaffolds, enzymes, growth factors, hormones, interferons, interleukins, and thrombolytics.

In some embodiments the polypeptide of interest is a receptor, such as an inducible receptor. Examples of suitable receptors include, but are not limited to, T cell receptors (TCRs), chimeric T cell receptors, artificial T cell receptors, synthetic T cell receptors, chimeric immunoreceptors, antibody-coupled T cell receptors (ACTRs), T cell receptor fusion constructs (TRUCs), and chimeric antigen receptors (CARs).

In some embodiments the polypeptide of interest is a cytokine, such as a proinflammatory cytokine or an anti-inflammatory cytokine. Examples of suitable cytokines include, but are not limited to, IL-2, IL-7, IL-12, IL-15, IL-18, and IL-21.

### Inducible Receptors

In one aspect, a polypeptide of interest of the present disclosure is an inducible cell receptor, which comprises an extracellular protein binding domain, a first intracellular signaling domain, and a transmembrane domain located between the extracellular protein binding domain and the first intracellular signaling domain; and a operably linked to the fusion protein. In another aspect, a polypeptide of interest of the present disclosure is an inducible cell receptor comprising (a) an extracellular protein binding domain, (b) a first intracellular signaling domain, and (c) a transmembrane domain located between the extracellular protein binding domain and the first intracellular signaling domain.

### ON and OFF Switches

In some embodiments, the present disclosure provides a fusion protein with an “OFF switch,” wherein the polypeptide of interest is an inducible receptor that is selectively inactivated in the presence of a protease inhibitor. An exemplary OFF switch, as provided herein, may be a cell receptor that comprises (a) a molecular binding domain (e.g., an extracellular protein binding domain), (b) an intracellular signaling domain, (c) a transmembrane domain (e.g., located between the molecular binding domain and the signaling domain), and (d) a, wherein components (a)-d) are configured such that the cell receptor is inactivated (does not transmit an intracellular signal) when the repressible protease is repressed. In some embodiments, the is located at the C-terminal (carboxy-terminal) end of the polypeptide of interest, at the N-terminal (amino-terminal) end of the polypeptide of interest, or located within domains of the polypeptide of interest. With OFF switches, cleavage by the protease removes the, thereby preserving structural integrity of the receptor, and addition of the protease inhibitor causes degradation of the receptor.

In some embodiments, the present disclosure provides a fusion protein with an “ON switch,” wherein the polypeptide of interest is an inducible receptor that is selectively activated in the presence of a protease inhibitor. An exemplary ON switch, as provided herein, may be a cell receptor that comprises (a) a molecular binding domain (e.g., an extracellular protein binding domain), (b) a signaling domain, (c) a transmembrane domain (e.g., located between the molecular binding domain and the signaling domain), (d) a protease, and (e) a cognate cleavage site, wherein components (a)-(e) are configured such that the cell receptor is activated (transmits an intracellular signal) when the protease is repressed. Unlike the OFF switches above, the ON switches do not include a. Rather, with ON switches, cleavage by the protease removes a functional element of the cell receptor (e.g., a signaling domain or a protein-binding domain), and addition of the protease inhibitor preserves structural integrity of the receptor.

The protease and the cognate cleavage site of an ON switch may be located between any two domains of the cell receptor. For example, the protease and the cognate cleavage site may be located between the extracellular protein binding domain and the transmembrane domain. In some embodiments, the protease and the cognate cleavage site are located between the transmembrane domain and the intracellular signaling domain. In other embodiments, the protease and the cognate cleavage site are located between two co-signaling domains. In some embodiments, a domain of the cell receptor further comprises a ligand operably linked to the ligand-binding domain (e.g., an extracellular protein binding domain). In this case, the protease and the cognate cleavage site can be located between the ligand and the ligand-binding domain.

In some embodiments, the inducible cell receptor comprises two polypeptides (e.g., a multichain receptor). In such embodiments, recruitment domains can be used to bring the two polypeptides together to activate the receptor. Recruitment domains are protein domains that bind to each other and thus, can bring together two different polypeptides, each comprising one of a pair of recruitment domains. A pair of recruitment domains are considered to assemble with each other if the two domains bind directly to each other, or if the two domains bind to the same (intermediate) molecule. Non-limiting examples of pairs of recruitment domains include (a) FK506 binding protein (FKBP) and FKBP; (b) FKBP and calcineurin catalytic subunit A (CnA); (c) FKBP and cyclophilin; (d) FKBP and FKBP-rapamycin associated protein (FRB); (e) gyrase B (GyrB) and GyrB; (f) dihydrofolate reductase (DHFR) and DHFR, g) DmrB and DmrB; (g) PYL and ABI; (h) Cry2 and CIP; and (i) GAI and GID1.

In some embodiments of the OFF switches, one polypeptide comprises a protein binding domain, a transmembrane domain, a signaling domain, and a first recruitment domain. In some embodiments, the second polypeptide comprises a second recruitment domain that assembles with the first recruitment domain. In some embodiments, a is located in the first polypeptide or in the second polypeptide. In some embodiments, the protease may be located in one (a first) polypeptide, while the cognate cleavage site and are located in the other (a second) polypeptide.

In some embodiments of the ON switches, a first polypeptide may comprise a protein binding domain, a transmembrane domain, a signaling domain, a first recruitment domain, and the cognate cleavage site. In some embodiments, the second polypeptide comprises the protease and a second recruitment domain that assembles with (binds directly or indirectly to) the first recruitment domain.

Also provided herein are methods of regulating activity of a cell receptor (e.g., OFF switches). In some embodiments of the OFF switches, the methods comprise providing a cell comprising cell receptor that includes (a) an extracellular protein binding domain, (b) an intracellular signaling domain, (c) a transmembrane domain located between the protein binding domain and the signaling domain, (d) a, (e) a protease (e.g., NS3 protease), and (f) a cognate cleavage site, wherein components (a)-(f) are configured such that the cell receptor is inactivated when the protease is repressed, and contacting the cell with a protease inhibitor (e.g., simeprevir, danoprevir, asunaprevir, ciluprevir, boceprevir, sovaprevir, paritaprevir, telaprevir, grazoprevir, glecaprevir, or voxiloprevir) that represses activity of the protease, thereby inactivating the cell receptor.

In other embodiments of the ON switches, the methods comprise providing a cell comprising a cell receptor that includes (a) an extracellular protein binding domain, (b) an intracellular signaling domain, (c) a transmembrane domain located between the protein binding domain and the signaling domain, (d) a protease (e.g., NS3 protease), and (e) a cognate cleavage site, wherein components (a)-(e) are configured such that the cell receptor is activated when the repressible protease is repressed, and contacting the cell with a protease inhibitor (e.g., simeprevir, danoprevir, asunaprevir, ciluprevir, boceprevir, sovaprevir, paritaprevir, telaprevir, grazoprevir, glecaprevir, or voxiloprevir) that represses activity of the protease, thereby activating the cell receptor.

### Chimeric Antigen Receptors (CARs)

In one aspect, a polypeptide of interest of the present disclosure is a chimeric antigen receptor (CAR) CARs, generally, are artificial immune cell receptors engineered to recognize and bind to an antigen expressed by tumor cells. CARs may typically include an antibody fragment as an antigen-binding domain, a spacer domains, a hydrophobic alpha helix transmembrane domain, and one or more intracellular signaling/co-signaling domains, such as (but not limited to) CD3-zeta, CD28, 4-1BB and/or OX40. A CAR can include a signaling domain or at least two co-signaling domains. In some embodiments, a CAR includes three or four co-signaling domains. In some embodiments, a is located in the C-terminus of the CAR.

Generally, a CAR is designed for a T cell, or NK cell, and is a chimera of a signaling domain of the T-cell receptor (TCR) complex and an antigen-recognizing domain (e.g., a single chain fragment (scFv) of an antibody) (Enblad et al., Human Gene Therapy. 2015: 26(8):498-505). A T cell that expresses a CAR is known in the art as a CAR T cell.

There are at least four generations of CARs, each of which contains different components. First generation CARs join an antibody-derived scFv to the CD3zeta (ζ or z) intracellular signaling domain of the T-cell receptor through hinge and transmembrane domains. Second generation CARs incorporate an additional domain, e.g., CD28, 4-1BB (41BB), or ICOS, to supply a costimulatory signal. Third-generation CARs contain two costimulatory domains fused with the TcR CD3-ζ chain. Third-generation costimulatory domains may include, e.g., a combination of CD3z, CD27, CD28, 4-1BB, ICOS, or OX40. CARs, in some embodiments, contain an ectodomain (e.g., CD3ζ), commonly derived from a single chain variable fragment (scFv), a hinge, a transmembrane domain, and an endodomain with one (first generation), two (second generation), or three (third generation) signaling domains derived from CD3Z and/or co-stimulatory molecules (Maude et al., Blood 2015, 125(26):4017-4023, Kakarla and Gottschalk, Cancer J. 2014; 20(2):151-155).

In some embodiments, a chimeric antigen receptor (CAR) is a T-cell redirected for universal cytokine killing (TRUCK), also known as a fourth generation CAR. TRUCKs are CAR-redirected T-cells used as vehicles to produce and release a transgenic cytokine that accumulates in the targeted tissue, e.g., a targeted tumor tissue. The transgenic cytokine is released upon CAR engagement of the target. TRUCK cells may deposit a variety of therapeutic cytokines in the target. This may result in therapeutic concentrations at the targeted site and avoid systemic toxicity.

CARs typically differ in their functional properties. The CD3ζ signaling domain of the T-cell receptor, when engaged, will activate and induce proliferation of T-cells but can lead to anergy (a lack of reaction by the body's defense mechanisms, resulting in direct induction of peripheral lymphocyte tolerance). Lymphocytes are considered anergic when they fail to respond to a specific antigen. The addition of a costimulatory domain in second-generation CARs improved replicative capacity and persistence of modified T-cells. Similar antitumor effects are observed in vitro with CD28 or 4-1BB CARs, but preclinical in vivo studies suggest that 4-1BB CARs may produce superior proliferation and/or persistence. Clinical trials suggest that both of these second-generation CARs are capable of inducing substantial T-cell proliferation in vivo, but CARs containing the 4-1BB costimulatory domain appear to persist longer. Third generation CARs combine multiple signaling domains (costimulatory) to augment potency. Fourth generation CARs are additionally modified with a constitutive or inducible expression cassette for a transgenic cytokine, which is released by the CAR T-cell to modulate the T-cell response. See, for example, Enblad et al., Human Gene Therapy. 2015; 26(8):498-505; Chmielewski and Hinrich, Expert Opinion on Biological Therapy 2015; 15(8) 1145-1154.

In some embodiments, a chimeric antigen receptor of the present disclosure is a first generation CAR. In some embodiments, a chimeric antigen receptor of the present disclosure is a second generation CAR. In some embodiments, a chimeric antigen receptor of the present disclosure is a third generation CAR. In some embodiments, a chimeric antigen receptor of the present disclosure is a fourth generation CAR.

In some embodiments, a spacer domain or a hinge domain is located between an extracellular domain (e.g., comprising the antigen binding domain) and a transmembrane domain of a CAR, or between a cytoplasmic signaling domain and a transmembrane domain of the CAR. A spacer domain is any oligopeptide or polypeptide that functions to link the transmembrane domain to the extracellular domain and/or the cytoplasmic signaling domain in the polypeptide chain. A hinge domain is any oligopeptide or polypeptide that functions to provide flexibility to the CAR, or domains thereof, or to prevent steric hindrance of the CAR, or domains thereof. In some embodiments, a spacer domain or hinge domain may comprise up to 300 amino acids (e.g., 10 to 100 amino acids, or 5 to 20 amino acids). In some embodiments, one or more spacer domain(s) may be included in other regions of a CAR.

In some embodiments, a CAR is an antigen-specific inhibitory CAR (iCAR), which may be used, for example, to avoid off-tumor toxicity (Fedorov, V D et al. Sci. Transl. Med. 2013, incorporated herein by reference). iCARs contain an antigen-specific inhibitory receptor, for example, to block nonspecific immunosuppression, which may result from extra-tumor target expression. iCARs may be based, for example, on inhibitory molecules CTLA-4 or PD-1. In some embodiments, these iCARs block T cell responses from T cells activated by either their endogenous T cell receptor or an activating CAR. In some embodiments, this inhibiting effect is temporary.

In some embodiments, CARs may be used in adoptive cell transfer, wherein immune cells are removed from a subject and modified so that they express receptors specific to an antigen, e.g., a tumor-specific antigen. The modified immune cells, which may then recognize and kill the cancer cells, are reintroduced into the subject (Pule, et al., Cytotherapy. 2003; 5(3): 211-226: Maude et al., Blood. 2015; 125(26). 4017-4023, each of which is incorporated herein by reference).

### Multipart CARs

In some embodiments, a polypeptide of interest of the present disclosure is a single chain (polypeptide) cell receptor or a multichain (and thus multipart) receptor. Thus, an ON switch or an OFF switch may comprise a single polypeptide, or at least two polypeptides.

In some embodiments of an OFF switch, a CAR is a multipart receptor comprising at least two polypeptides. In some embodiments, the CAR comprises a first polypeptide comprising (a) an extracellular protein binding domain (e.g., an antibody fragment), (b) a signaling domain, (c) a transmembrane domain located between the extracellular protein binding domain and the signaling domain, and (d) a first recruitment domain, and a second polypeptide comprising a signaling domain and a second recruitment domain that assembles with the first recruitment domain, wherein a is located in the first polypeptide and/or the second polypeptide. In some embodiments, the is located in the C-terminus of the first polypeptide and/or the second polypeptide.

In other embodiments of an OFF switch, the CAR comprises a first polypeptide comprising (a) an extracellular protein binding domain (e.g., an antibody fragment), (b) a signaling domain, (c) a transmembrane domain located between the an extracellular protein binding domain and the signaling domain, and (d) a first recruitment domain, and a second polypeptide comprising a second recruitment domain that assembles with the first recruitment domain, wherein the protease is located in the first polypeptide, and the cognate cleavage site and a are located in the second polypeptide, or wherein the protease is located in the second polypeptide, and the cognate cleavage site and are located in the first polypeptide. In some embodiments, the is located in the C-terminus of the first polypeptide and/or the second polypeptide.

In some embodiments of an ON switch, a CAR comprises a first polypeptide comprising (a) an extracellular protein binding domain (e.g., an antibody fragment), (b) a first intracellular signaling domain, (c) a transmembrane domain located between the antibody fragment and the intracellular signaling domain, (d) a second intracellular signaling domain, and (d) a first recruitment domain; and a second polypeptide comprising the protease and a second recruitment domain that assembles with the first recruitment domain, wherein the cognate cleavage site is located between the antibody fragment and the transmembrane domain, between the transmembrane domain and first intracellular signaling domain, or between the first intracellular signaling domain and the second intracellular signaling domain.

In other embodiments of an ON switch, a CAR comprises a first polypeptide comprising (a) an extracellular protein binding domain (e.g., an antibody fragment), (b) a first intracellular signaling domain, (c) a transmembrane domain located between the antibody fragment and the intracellular signaling domain, (d) a second intracellular signaling domain, and (d) a first recruitment domain; and a second polypeptide comprising the protease and a second recruitment domain that assembles with the first recruitment domain, wherein the cognate cleavage site is located between the antibody fragment and the transmembrane domain, between the transmembrane domain and first intracellular signaling domain, or between the first intracellular signaling domain and the second intracellular signaling domain.

Additional CAR-Regulation Switches

In some embodiments, a (e.g., OFF switch) and/or a protease/cognate cleavage site (e.g., ON switch) may be combined with orthogonal CAR-regulating switches to yield logic gates (e.g., AND, OR, NOR, and conditional ON gates) with, for example, at least 2 agent (e.g., drug) inputs that perform higher order functionalities.

In some embodiments, a CAR comprises a first polypeptide comprising (a) an extracellular protein binding domain (e.g., an antibody fragment), (b) a signaling domain, (c) a transmembrane domain located between the extracellular protein binding domain and the signaling domain, (d) a first recruitment domain, (e) a, (f) a protease, and (g) a cognate cleavage site, and a second polypeptide comprising a signaling domain and a second recruitment domain that assembles with the first recruitment domain only when the CAR is contacted with an agent required for assembly of the first recruitment domain with the second recruitment domain. In some embodiments, methods of regulating activity of the CAR comprise contacting a cell comprising the CAR with (a) a protease inhibitor that represses activity of the protease and (b) an agent required for assembly of the first recruitment domain with the second recruitment domain, thereby activating the CAR.

In other embodiments, a CAR comprises a first polypeptide comprising (a) an extracellular protein binding domain (e.g., an antibody fragment), (b) a signaling domain, (c) a transmembrane domain located between the antibody fragment and the signaling domain, (d) a first recruitment domain, (e) a, (f) a protease, and (g) a cognate cleavage site, and a second polypeptide comprising a signaling domain and a second recruitment domain that assembles with the first recruitment domain unless in the CAR is contacted with an agent that prevents assembly of the first recruitment domain with the second recruitment domain. In some embodiments, methods of regulating activity of the CAR comprise contacting a cell comprising the CAR with (a) a protease inhibitor that represses activity of the protease and (b) an agent that prevents assembly of the first recruitment domain with the second recruitment domain, thereby inactivating the CAR.

In yet other embodiments, a CAR comprises a first polypeptide comprising (a) an antibody fragment, (b) a signaling domain, (c) a transmembrane domain located between the antibody fragment and the signaling domain, (d) a first recruitment domain, and (e) a protease and a cognate cleavage site, wherein the protease and cognate cleavage site are located between the signaling domain and the first recruitment domain, and a second polypeptide comprising a signaling domain and a second recruitment domain that assembles with the first recruitment domain only when the CAR is contacted with an agent required for assembly of the first recruitment domain with the second recruitment domain. In some embodiments, methods of regulating activity of the CAR comprise contacting a cell comprising the CAR with (a) a protease inhibitor that represses activity of the protease and (b) an agent required for assembly of the first recruitment domain with the second recruitment domain, thereby activating the CAR.

In still other embodiments, a CAR comprises a first polypeptide comprising (a) an antibody fragment, (b) a signaling domain, (c) a transmembrane domain located between the antibody fragment and the signaling domain, and (d) a first recruitment domain, and a second polypeptide comprising a second recruitment domain that assembles with the first recruitment domain only when the CAR is contacted with an agent required for assembly of the first recruitment domain with the second recruitment domain, wherein the CAR further comprises a, a protease, a cognate cleavage site, and wherein the cognate cleavage site and are located at the C-terminus of the first polypeptide and the protease is located at the C-terminus of the second polypeptide. In some embodiments, methods of regulating activity of the CAR comprise contacting a cell comprising the CAR with an agent required for assembly of the first recruitment domain with the second recruitment domain, thereby activating the CAR. The methods may further comprise contacting the cell with a protease inhibitor that represses activity of the protease, thereby inactivating the CAR.

In some embodiments, a CAR comprises a first polypeptide comprising (a) an antibody fragment, (b) a signaling domain, (c) a transmembrane domain located between the antibody fragment and the signaling domain, (d) a first recruitment domain, (e) an inhibitory domain, and (f) a protease and cognate cleavage site located between the first recruitment domain and the inhibitory domain, and a second polypeptide comprising a second recruitment domain that assembles with the first recruitment domain only when the CAR is contacted with an agent required for assembly of the first recruitment domain with the second recruitment domain. In some embodiments, methods of regulating activity of the CAR comprise contacting a cell comprising the CAR with an agent required for assembly of the first recruitment domain with the second recruitment domain, thereby activating the CAR The methods may further comprise contacting the cell with a protease that represses activity of the protease, thereby inactivating the CAR.

The ability of constructs to produce fusion proteins can be empirically determined (e.g., detecting fusion proteins labeled with EGFP or AIA by fluorescence microscopy or immunoblotting, respectively).

Additionally, production and, in certain embodiments, the degradation ofa polypeptide of interest in the presence and absence of protease inhibitors can be monitored. Because the presence of a protease inhibitor prevents accumulation of new protein copies without affecting old copies, the overall levels of a polypeptide of interest after adding the protease inhibitor depend on its degradation rate. Accordingly, the half-life of the polypeptide of interest in a cell can be readily calculated by monitoring its decay. Additionally, the turnover of the polypeptide of interest can be determined by measuring amounts of the polypeptide of interest in a transformed cell before and after contacting the cell with a protease inhibitor and calculating the turnover of the polypeptide of interest based on the amounts of the polypeptide of interest in the cell before and after adding the protease inhibitor. The amount of the polypeptide of interest in the cell can be measured either continuously or periodically over a period of time by any suitable method (e.g., immunoblotting or microscopy).

### Production of Fusion Proteins

Fusion proteins of the present disclosure can be produced using recombinant techniques well known in the art. One of skill in the art can readily determine nucleotide sequences that encode the desired polypeptides using standard methodology and the teachings herein. Oligonucleotide probes can be devised based on the known sequences and used to probe genomic or cDNA libraries. The sequences can then be further isolated using standard techniques and, e.g., restriction enzymes employed to truncate the gene at desired portions of the full-length sequence. Similarly, sequences of interest can be isolated directly from cells and tissues containing the same, using known techniques, such as phenol extraction and the sequence further manipulated to produce the desired truncations See, e.g., Sambrook et al., supra, for a description of techniques used to obtain and isolate DNA.

The sequences encoding polypeptides can also be produced synthetically, for example, based on the known sequences. The nucleotide sequence can be designed with the appropriate codons for the particular amino acid sequence desired. The complete sequence is generally assembled from overlapping oligonucleotides prepared by standard methods and assembled into a complete coding sequence. See, e.g., Edge (1981) Nature 292 756: Nambair et al. (1984) Science 223:1299; Jay et al (1984) J. Biol. Chem. 259:6311, Stemmer et al. (1995) Gene 164:49-53.

Recombinant techniques are readily used to clone sequences encoding polypeptides useful in the claimed fusion proteins that can then be mutagenized in vitro by the replacement of the appropriate base pair(s) to result in the codon for the desired amino acid. Such a change can include as little as one base pair, effecting a change in a single amino acid, or can encompass several base pair changes.

Alternatively, the mutations can be affected using a mismatched primer that hybridizes to the parent nucleotide sequence (generally cDNA corresponding to the RNA sequence), at a temperature below the melting temperature of the mismatched duplex. The primer can be made specific by keeping primer length and base composition within relatively narrow limits and by keeping the mutant base centrally located. See, e.g., Innis et al, (1990) PCR Applications. Protocols for Functional Genomics; Zoller and Smith, Methods Enzymol. (1983) 100:468. Primer extension is affected using DNA polymerase, the product cloned and clones containing the mutated DNA, derived by segregation of the primer extended strand, selected.

Selection can be accomplished using the mutant primer as a hybridization probe. The technique is also applicable for generating multiple point mutations. See, e.g., Dalbie-McFarland et al. Proc. Natl. Acad. Sci USA (1982) 79:6409.

Once coding sequences have been isolated and/or synthesized, they can be cloned into any suitable vector or replicon for expression. As will be apparent from the teachings herein, a wide variety of vectors encoding modified polypeptides can be generated by creating expression constructs which operably link, in various combinations, polynucleotides encoding polypeptides having deletions or mutations therein.

Numerous cloning vectors are known to those of skill in the art, and the selection of an appropriate cloning vector is a matter of choice. Examples of recombinant DNA vectors for cloning and host cells which they can transform include the bacteriophage λ (E. coli), pBR322 (E. coli), pACYC177 (E. coli), pKT230 (gram-negative bacteria), pGVI 106 (gram-negative bacteria), pLAFR1 (gram-negative bacteria), pME290 (non-E. coli gram-negative bacteria), pHV14 (E. coli and Bacillus subtilis), pBD9 (Bacillus), pU61 (Streptomyces), pUC6 (Streptomyces), YIp5 (Saccharomyces), YCp19 (Saccharomyces) and bovine papilloma virus (mammalian cells) See, generally, DNA Cloning Vols I & II, supra; Sambrook et al, supra; B. Perbal, supra.

Insect cell expression systems, such as baculovirus systems, can also be used and are known to those of skill in the art and described in, e.g., Summers and Smith, Texas Agricultural Experiment Station Bulletin No. 1555 (1987). Materials and methods for baculovirus/insect cell expression systems are commercially available in kit form from, inter alia, Invitrogen, San Diego Calif. (“MaxBac” kit).

Plant expression systems can also be used to produce the fusion proteins described herein. Generally, such systems use virus-based vectors to transfect plant cells with heterologous genes. For a description of such systems see, e.g., Porta et al., Mol. Biotech (1996) 5:209-221; and Hackland et al., Arch. Virol. (1994) 139: 1-22.

Viral systems, such as a vaccinia based infection/transfection system, as described in Tomei et al., J. Virol. (1993) 67:4017-4026 and Selby et al., J. Gen. Virol. (1993) 74: 1103-1113, will also find use with the present disclosure. In this system, cells are first transfected in vitro with a vaccinia virus recombinant that encodes the bacteriophage T7 RNA polymerase. This polymerase displays exquisite specificity in that it only transcribes templates bearing T7 promoters. Following infection, cells are transfected with the DNA of interest, driven by a T7 promoter. The polymerase expressed in the cytoplasm from the vaccinia virus recombinant transcribes the transfected DNA into RNA that is then translated into protein by the host translational machinery. The method provides for high level, transient, cytoplasmic production of large quantities of RNA and its translation product(s).

The gene can be placed under the control of a promoter, ribosome binding site (for bacterial expression) and, optionally, an operator (collectively referred to herein as “control” elements), so that the DNA sequence encoding the desired polypeptide is transcribed into RNA in the host cell transformed by a vector containing this expression construction. The coding sequence may or may not contain a signal peptide or leader sequence. With the present disclosure, both the naturally occurring signal peptides and heterologous sequences can be used. Leader sequences can be removed by the host in post-translational processing. See, e.g., U.S. Pat. Nos. 4,431,739; 4,425,437, 4,338,397 Such sequences include, but are not limited to, the TPA leader, as well as the honeybee mellitin signal sequence.

Other regulatory sequences may also be desirable which allow for regulation of expression of the protein sequences relative to the growth of the host cell Such regulatory sequences are known to those of skill in the art, and examples include those which cause the expression of a gene to be turned on or off in response to a chemical or physical stimulus, including the presence of a regulatory compound. Other types of regulatory elements may also be present in the vector, for example, enhancer sequences.

The control sequences and other regulatory sequences may be ligated to the coding sequence prior to insertion into a vector. Alternatively, the coding sequence can be cloned directly into an expression vector that already contains the control sequences and an appropriate restriction site.

In some cases, it may be necessary to modify the coding sequence so that it may be attached to the control sequences with the appropriate orientation; i.e., to maintain the proper reading frame. Mutants or analogs may be prepared by the deletion of a portion of the sequence encoding the protein, by insertion of a sequence, and/or by substitution of one or more nucleotides within the sequence. Techniques for modifying nucleotide sequences, such as site-directed mutagenesis, are well known to those skilled in the art. See, e.g., Sambrook et al, supra; DNA Cloning, Vols I and II, supra; Nucleic Acid Hybridization, supra.

The expression vector is then used to transform an appropriate host cell. A number of mammalian cell lines are known in the art and include immortalized cell lines available from the American Type Culture Collection (ATCC), such as, but not limited to, Chinese hamster ovary (CHO) cells, HeLa cells, baby hamster kidney (BHK) cells, monkey kidney cells (COS), human hepatocellular carcinoma cells {e.g., Hep G2), Vero293 cells, as well as others. Similarly, bacterial hosts such as E. coli, Bacillus subtilis, and Streptococcus spp., will find use with the present expression constructs. Yeast hosts useful in the present disclosure include inter alia, Saccharomyces cerevisiae, Candida albicans, Candida maltosa, Hansenula polymorpha, Kluyveromyces fragilis, Kluyveromyces lactis, Pichia guillerimondii, Pichia pastoris, Schizosaccharomyces pombe and Yarrowia lipolytica. Insect cells for use with baculovirus expression vectors include, inter alia, Aedes aegypti, Autographa calif or nica, Bombyx mori, Drosophila melanogaster, Spodoptera frugiperda, and Trichoplusia ni.

Depending on the expression system and host selected, the fusion proteins of the present disclosure are produced by growing host cells transformed by an expression vector described above under conditions whereby the protein of interest is expressed. The selection of the appropriate growth conditions is within the skill of the art.

In one embodiment, the transformed cells secrete the polypeptide product into the surrounding media Certain regulatory sequences can be included in the vector to enhance secretion of the protein product, for example using a tissue plasminogen activator (TP A) leader sequence, an interferon (y or a) signal sequence or other signal peptide sequences from known secretory proteins. The secreted polypeptide product can then be isolated by various techniques described herein, for example, using standard purification techniques such as but not limited to, hydroxy apatite resins, column chromatography, ion-exchange chromatography, size-exclusion chromatography, electrophoresis, HPLC, immunoadsorbent techniques, affinity chromatography, immunoprecipitation, and the like. Alternatively, the transformed cells are disrupted, using chemical, physical or mechanical means, which lyse the cells yet keep the recombinant polypeptides substantially intact. Intracellular proteins can also be obtained by removing components from the cell wall or membrane, e.g., by the use of detergents or organic solvents, such that leakage of the polypeptides occurs. Such methods are known to those of skill in the art and are described in, e.g., Protein Purification Applications: A Practical Approach, (Simon Roe, Ed., 2001).

For example, methods of disrupting cells for use with the present disclosure include but are not limited to: sonication or ultrasonication; agitation; liquid or solid extrusion; heat treatment; freeze-thaw; desiccation; explosive decompression, osmotic shock; treatment with lytic enzymes including proteases such as trypsin, neuraminidase and lysozyme; alkali treatment; and the use of detergents and solvents such as bile salts, sodium dodecyl sulphate, Triton, P40 and CHAPS. The particular technique used to disrupt the cells is largely a matter of choice and will depend on the cell type in which the polypeptide is expressed, culture conditions and any pre-treatment used.

Following disruption of the cells, cellular debris is removed, generally by centrifugation, and the intracellularly produced polypeptides are further purified, using standard purification techniques such as but not limited to, column chromatography, ion-exchange chromatography, size-exclusion chromatography, electrophoresis, FIPLC, immunoadsorbent techniques, affinity chromatography, immunoprecipitation, and the like.

For example, one method for obtaining the intracellular polypeptides of the present disclosure involves affinity purification, such as by immunoaffinity chromatography using antibodies (e.g., previously generated antibodies), or by lectin affinity chromatography. Particularly preferred lectin resins are those that recognize mannose moieties such as but not limited to resins derived from Galanthus nivalis agglutinin (GNA), Lens culinaris agglutinin (LCA or lentil lectin), Pisum sativum agglutinin (PSA or pea lectin), Narcissus pseudonarcissus agglutinin (PA) and Allium ursinum agglutinin (AUA). The choice of a suitable affinity resin is within the skill in the art. After affinity purification, the polypeptides can be further purified using conventional techniques well known in the art, such as by any of the techniques described above.

### Polynucleotides Encoding Fusion Proteins

In another aspect, the present disclosure provides a polynucleotide encoding a fusion protein of the present disclosure, and a vector comprising such a polynucleotide. In some embodiments, the polynucleotide comprises a sequence encoding an inducible cell receptor (e.g., a CAR), wherein the sequence encoding an extracellular protein binding domain is contiguous with and in the same reading frame as a sequence encoding an intracellular signaling domain and a transmembrane domain.

The polynucleotide can be codon optimized for expression in a mammalian cell in some embodiments, the entire sequence of the polynucleotide has been codon optimized for expression in a mammalian cell. Codon optimization refers to the discovery that the frequency of occurrence of synonymous codons (i.e., codons that code for the same amino acid) in coding DNA is biased in different species. Such codon degeneracy allows an identical polypeptide to be encoded by a variety of nucleotide sequences. A variety of codon optimization methods is known in the art, and include, e.g., methods disclosed in at least U.S. Pat. Nos. 5,786,464 and 6,114,148

The polynucleotide encoding a fusion protein can be obtained using recombinant methods known in the art, such as, for example by screening libraries from cells expressing the polynucleotide, by deriving it from a vector known to include the same, or by isolating directly from cells and tissues containing the same, using standard techniques. Alternatively, the polynucleotide can be produced synthetically, rather than cloned.

The polynucleotide can be cloned into a vector. In some embodiments, an expression vector known in the art is used. For example, polynucleotide described herein can be inserted into an expression vector to create an expression cassette capable of producing the degron fusion proteins in a suitable host cell (e.g. in a tissue, organ, organoid, or subject). Expression cassettes typically include control elements operably linked to the coding sequence, which allow for the expression of the gene in vivo in the subject species. For example, typical promoters for mammalian cell expression include the SV40 early promoter, a CMV promoter such as the CMV immediate early promoter, the mouse mammary tumor virus LTR promoter, the adenovirus major late promoter (Ad MLP), and the herpes simplex virus promoter, among others. Other nonviral promoters, such as a promoter derived from the murine metallothionein gene, will also find use for mammalian expression. Typically, transcription termination and polyadenylation sequences will also be present, located 3′ to the translation stop codon Preferably, a sequence for optimization of initiation of translation, located 5′ to the coding sequence, is also present. Examples of transcription terminator/polyadenylation signals include those derived from SV40, as described in Sambrook et al., supra, as well as a bovine growth hormone terminator sequence.

Enhancer elements may also be used herein to increase expression levels of mammalian constructs. Examples include the SV40 early gene enhancer, as described in Dijkema et al., EMPO J. (1985) 4 761, the enhancer/promoter derived from the long terminal repeat (LTR) of the Rous Sarcoma Virus, as described in Gorman et al., Proc. Natl. Acad. Sci. USA (1982b) 79:6777 and elements derived from human CMV, as described in Boshart et al., Cell (1985) 41:521, such as elements included in the CMV intron A sequence.

Constructs encoding fusion proteins can be administered to a subject or introduced into cells, tissue, organs, or organoids using standard gene delivery protocols. Methods for gene delivery are known in the art. See, e.g., U.S. Pat. Nos. 5,399,346, 5,580,859, 5,589,466. Genes can be delivered either directly to a subject or, alternatively, delivered ex vivo, to cells derived from the subject and the cells reimplanted in the subject.

A number of viral based systems have been developed for gene transfer into mammalian cells. These include adenoviruses, retroviruses (y-retroviruses and lentiviruses), poxviruses, adeno-associated viruses, baculoviruses, and herpes simplex viruses (see e.g., Warnock et al. (2011) Methods Mol. Biol. 737: 1-25; Walther et al. (2000) Drugs 60(2):249-271; and Lundstrom (2003) Trends Biotechnol 21(3): 117-122, herein incorporated by reference).

For example, retroviruses provide a convenient platform for gene delivery systems. Selected sequences can be inserted into a vector and packaged in retroviral particles using techniques known in the art. The recombinant virus can then be isolated and delivered to cells of the subject either in vivo or ex vivo. A number of retroviral systems have been described (U.S. Pat. No. 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc Natl. Acad Sci. USA 90:8033-8037, Boris-Lawrie and Temin (1993) Cur. Opin. Genet. Develop. 3:102-109; and Ferry et al. (2011) Curr Pharm Des. 17(24):2516-2527). Lentiviruses are a class of retroviruses that are particularly useful for delivering polynucleotides to mammalian cells because they are able to infect both dividing and nondividing cells (see e.g., Lois et al (2002) Science 295:868-872; Durand et al. (2011) Viruses 3(2): 132-159; herein incorporated by reference).

A number of adenovirus vectors have also been described. Unlike retroviruses which integrate into the host genome, adenoviruses persist extrachromosomally thus minimizing the risks associated with insertional mutagenesis (Haj-Ahmad and Graham, J. Virol. (1986) 57:267-274: Bett et al., J. Virol (1993) 67:5911-5921, Mittereder et al., Human Gene Therapy (1994) 5:717-729: Seth et al., J. Virol. (1994) 68:933-940: Barr et al., Gene Therapy (1994) 1:51-58; Berkner, K. L. BioTechniques (1988) 6:616-629; and Rich et al., Human Gene Therapy (1993) 4:461-476).

Additionally, various adeno-associated virus (AAV) vector systems have been developed for gene delivery. AAV vectors can be readily constructed using techniques well known in the art. See, e.g., U.S. Pat. Nos. 5,173,414 and 5,139,941; International Publication Nos WO 92/01070 (published 23 Jan. 1992) and WO 93/03769 (published 4 Mar. 1993); Lebkowski et al., Molec. Cell. Biol. (1988) 8:3988-3996: Vincent et al., Vaccines 90 (1990) (Cold Spring Harbor Laboratory Press), Carter, B. J Current Opinion in Biotechnology (1992) 3:533-539; Muzyczka, N. Current Topics in Microbiol, and Immunol. (1992) 158:97-129; Kotin, R. M. Human Gene Therapy (1994) 5.793-801; Shelling and Smith, Gene Therapy (1994) 1: 165-169; and Zhou et al., J. Exp. Med. (1994) 179: 1867-1875.

Another vector system useful for delivering the polynucleotides of the present disclosure is the enterically administered recombinant poxvirus vaccines described by Small, Jr., P. A., et al. (U.S. Pat. No. 5,676,950, issued Oct. 14, 1997, herein incorporated by reference).

Additional viral vectors which will find use for delivering the nucleic acid molecules encoding the fusion proteins of the present disclosure include those derived from the pox family of viruses, including vaccinia virus and avian poxvirus. By way of example, vaccinia virus recombinants expressing the fusion proteins can be constructed as follows. The DNA encoding the particular fusion protein coding sequence is first inserted into an appropriate vector so that it is adjacent to a vaccinia promoter and flanking vaccinia DNA sequences, such as the sequence encoding thymidine kinase (TK) This vector is then used to transfect cells which are simultaneously infected with vaccinia. Homologous recombination serves to insert the vaccinia promoter plus the gene encoding the coding sequences of interest into the viral genome. The resulting TK-recombinant can be selected by culturing the cells in the presence of 5-bromodeoxyuridine and picking viral plaques resistant thereto.

Alternatively, avipoxviruses, such as the fowlpox and canarypox viruses, can also be used to deliver the genes. Recombinant avipox viruses, expressing immunogens from mammalian pathogens, are known to confer protective immunity when administered to non-avian species. The use of an avipox vector is particularly desirable in human and other mammalian species since members of the avipox genus can only productively replicate in susceptible avian species and therefore are not infective in mammalian cells. Methods for producing recombinant avipoxviruses are known in the art and employ genetic recombination, as described above with respect to the production of vaccinia viruses. See, e.g., WO 91/12882; WO 89/03429; and WO 92/03545.

Molecular conjugate vectors, such as the adenovirus chimeric vectors described in Michael et al., J. Biol. Chem. (1993) 268:6866-6869 and Wagner et al., Proc. Nati. Acad. Sci. USA (1992) 89.6099-6103, can also be used for gene delivery.

Members of the Alphavirus genus, such as, but not limited to, vectors derived from the Sindbis virus (SIN), Semliki Forest virus (SFV), and Venezuelan Equine Encephalitis virus (VEE), will also find use as viral vectors for delivering the polynucleotides of the present disclosure. For a description of Sindbis-virus derived vectors useful for the practice of the instant methods, see, Dubensky et al. (1996) J. Virol. 70:508-519; and International Publication Nos. WO 95/07995, WO 96/17072; as well as, Dubensky, Jr., T. W., et al., U.S. Pat. No. 5,843,723, issued Dec. 1, 1998, and Dubensky, Jr., T. W., U.S. Pat. No. 5,789,245, issued Aug. 4, 1998, both herein incorporated by reference Particularly preferred are chimeric alphavirus vectors comprised of sequences derived from Sindbis virus and Venezuelan equine encephalitis vims. See, e.g., Perri et al (2003) J. Virol. 77, 10394-10403 and International Publication Nos WO 02/099035, WO 02/080982, WO 01/81609, and WO 00/61772; herein incorporated by reference in their entireties.

A vaccinia based infection/transfection system can be conveniently used to provide for inducible, transient expression of the coding sequences of interest (for example, a fusion protein expression cassette) in a host cell. In this system, cells are first infected in vitro with a vaccinia virus recombinant that encodes the bacteriophage T7 RNA polymerase. This polymerase displays exquisite specificity in that it only transcribes templates bearing T7 promoters Following infection, cells are transfected with the polynucleotide of interest, driven by a T7 promoter. The polymerase expressed in the cytoplasm from the vaccinia virus recombinant transcribes the transfected DNA into RNA which is then translated into protein by the host translational machinery. The method provides for high level, transient, cytoplasmic production of large quantities of RNA and its translation products See, e.g., Elroy-Stein and Moss, Proc. Natl. Acad. Sci. USA (1990) 87:6743-6747; Fuerst et al., Proc. Natl. Acad. Sci. USA (1986) 83:8122-8126.

As an alternative approach to infection with vaccinia or avipox virus recombinants, or to the delivery of genes using other viral vectors, an amplification system can be used that will lead to high level expression following introduction into host cells. Specifically, a T7 RNA polymerase promoter preceding the coding region for T7 RNA polymerase can be engineered. Translation of RNA derived from this template will generate T7 RNA polymerase which in turn will transcribe more template. Concomitantly, there will be a cDNA whose expression is under the control of the T7 promoter. Thus, some of the T7 RNA polymerase generated from translation of the amplification template RNA will lead to transcription of the desired gene. Because some T7 RNA polymerase is required to initiate the amplification, T7 RNA polymerase can be introduced into cells along with the template(s) to prime the transcription reaction. The polymerase can be introduced as a protein or on a plasmid encoding the RNA polymerase. For a further discussion of T7 systems and their use for transforming cells, see, e.g., International Publication No WO 94/26911; Studier and Moffatt, J Mol. Biol. (1986) 189: 113-130; Deng and Wolff, Gene (1994) 143:245-249; Gao et al., Biochem. Biophys. Res. Commun. (1994) 200: 1201-1206; Gao and Huang, Nuc. Acids Res. (1993) 21:2867-2872; Chen et al., Nuc. Acids Res (1994) 22:2114-2120; and U.S. Pat. No. 5,135,855.

The synthetic expression cassette of interest can also be delivered without a viral vector. For example, the synthetic expression cassette can be packaged as DNA or RNA in liposomes prior to delivery to the subject or to cells derived therefrom. Lipid encapsulation is generally accomplished using liposomes which are able to stably bind or entrap and retain nucleic acid. The ratio of condensed DNA to lipid preparation can vary but will generally be around 1:1 (mg DNA:micromoles lipid), or more of lipid. For a review of the use ofliposomes as carriers for delivery of nucleic acids, see, e.g., Hug and Sleight, Biochim. Biophys. Acta (1991) 1097: 1-17, Straubinger et al, in Methods of Enzymology (1983), Vol 101, pp 512-527.

Liposomal preparations for use in the present disclosure include cationic (positively charged), anionic (negatively charged) and neutral preparations, with cationic liposomes particularly preferred. Cationic liposomes have been shown to mediate intracellular delivery of plasmid DNA (Feigner et al., Proc. Natl. Acad. Sci. USA (1987) 84:7413-7416): mRNA (Malone et al., Proc. Natl. Acad. Sci. USA (1989) 86.6077-6081); and purified transcription factors (Debs et al., J. Biol. Chem. (1990) 265: 10189-10192), in functional form.

Cationic liposomes are readily available. For example, N[1-2,3-dioleyloxy)propyl]-N,N,N-triethylammonium (DOTMA) liposomes are available under the trademark Lipofectin, from GIBCO BRL, Grand Island, N Y (See, also, Feigner et al., Proc Natl Acad. Sci. USA (1987) 84:7413-7416). Other commercially available lipids include (DDAB/DOPE) and DOTAP/DOPE (Boerhinger). Other cationic liposomes can be prepared from readily available materials using techniques well known in the art. See, e.g., Szoka et al., Proc. Natl. Acad. Sci. USA (1978) 75:4194-4198; PCT Publication No. WO 90/11092 for a description of the synthesis of DOTAP (1,2-bis(oleoyloxy)-3-(trimethylammonio)propane) liposomes.

Similarly, anionic and neutral liposomes are readily available, such as, from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios Methods for making liposomes using these materials are well known in the art.

The liposomes can comprise multilammelar vesicles (MLVs), small unilamellar vesicles (SUVs), or large unilamellar vesicles (LUVs). The various liposome-nucleic acid complexes are prepared using methods known in the art See, e.g., Straubinger et al., in METHODS OF IMMUNOLOGY (1983), Vol. 101, pp. 512-527, Szoka et al., Proc. Natl. Acad. Sci. USA (1978) 75:4194-4198; Papahadjopoulos et al., Biochim. Biophys. Acta (1975) 394:483; Wilson et al, Cell (1979) 17.77); Deamer and Bangham, Biochim. Biophys. Acta (1976) 443:629; Ostro et al., Biochem. Biophys. Res. Commun. (1977) 76:836; Fraley et al., Proc. Natl. Acad. Sci. USA (1979) 76.3348); Enoch and Strittmatter, Proc. Natl Acad. Sci. USA (1979) 76: 145); Fraley et al., J. Biol. Chem. (1980) 255: 10431; Szoka and Papahadjopoulos, Proc. Natl. Acad. Sci. USA (1978) 75: 145, and Schaefer-Ridder et al., Science (1982) 215: 166.

The DNA and/or peptide(s) can also be delivered in cochleate lipid compositions similar to those described by Papahadjopoulos et al., Biochem. Biophys. Acta (1975) 394:483-491. See, also, U.S. Pat. Nos. 4,663,161 and 4,871,488.

The expression cassette of interest may also be encapsulated, adsorbed to, or associated with, particulate carriers Examples of particulate carriers include those derived from polymethyl methacrylate polymers, as well as microparticles derived from poly(lactides) and poly(lactide-co-glycolides), known as PLG. See, e.g., Jeffery et al., Pharm. Res (1993) 10.362-368; McGee J. P., et al., J Microencapsul. 14(2): 197-210, 1997; O'Hagan D. T., et al., Vaccine 11(2): 149-54, 1993.

Furthermore, other particulate systems and polymers can be used for the in vivo or ex vivo delivery of the nucleic acid of interest. For example, polymers such as polylysine, polyarginine, polyornithine, spermine, spermidine, as well as conjugates of these molecules, are useful for transferring a nucleic acid of interest. Similarly, DEAE dextran-mediated transfection, calcium phosphate precipitation or precipitation using other insoluble inorganic salts, such as strontium phosphate, aluminum silicates including bentonite and kaolin, chromic oxide, magnesium silicate, talc, and the like, will find use with the present methods. See, e.g., Feigner, P. L., Advanced Drug Delivery Reviews (1990) 5: 163-187, for a review of delivery systems useful for gene transfer Peptoids (Zuckerman, R N., et al., U.S. Pat. No. 5,831,005, issued Nov. 3, 1998, herein incorporated by reference) may also be used for delivery of a construct of the present disclosure.

Additionally, biolistic delivery systems employing particulate carriers such as gold and tungsten are especially useful for delivering synthetic expression cassettes of the present disclosure. The particles are coated with the synthetic expression cassette(s) to be delivered and accelerated to high velocity, generally under a reduced atmosphere, using a gun powder discharge from a “gene gun.” For a description of such techniques, and apparatuses useful therefore, see, e.g., U.S. Pat. Nos. 4,945,050; 5,036,006; 5,100,792; 5,179,022; 5,371,015; and 5,478,744 Also, needle-less injection systems can be used (Davis, H. L., et al, Vaccine 12 1503-1509, 1994; Bioject, Inc., Portland. Oreg.).

Recombinant vectors can be formulated into compositions for delivery to a vertebrate subject. The compositions will generally include one or more “pharmaceutically acceptable excipients or vehicles” such as water, saline, glycerol, polyethyleneglycol, hyaluronic acid, ethanol, etc. Additionally, auxiliary substances, such as wetting or emulsifying agents. pH buffering substances, surfactants and the like, may be present in such vehicles. Certain facilitators of nucleic acid uptake and/or expression can also be included in the compositions or coadministered.

Once formulated, the compositions of the present disclosure can be administered directly to the subject (e.g., as described above) or, alternatively, delivered ex vivo, to cells derived from the subject, using methods such as those described above. For example, methods for the ex vivo delivery and reimplantation of transformed cells into a subject are known in the art and can include, e.g., dextran-mediated transfection, calcium phosphate precipitation, polybrene mediated transfection, lipofectamine and LT-1 mediated transfection, protoplast fusion, electroporation, encapsulation of the polynucleotide(s) in liposomes, and direct microinjection of the DNA into nuclei.

Direct delivery of synthetic expression cassette compositions in vivo will generally be accomplished with or without viral vectors, as described above, by injection using either a conventional syringe, needless devices such as Bioject™ or a gene gun, such as the Accell gene delivery system (PowderMed Ltd, Oxford, England).

The present disclosure also includes an RNA construct that can be directly transfected into a cell. A method for generating mRNA for use in transfection involves in vitro transcription (IVT) of a template with specially designed primers, followed by polyA addition, to produce a construct containing 3′ and 5′ untranslated sequence (“UTR”) (e.g., a 3′ and/or 5′ UTR described herein), a 5′ cap (e.g., a 5′ cap described herein) and/or Internal Ribosome Entry Site (IRES) (e.g., an IRES described herein), the nucleic acid to be expressed, and a polyA tail. RNA so produced can efficiently transfect different kinds of cells.

### Cells

In one aspect, the present disclosure provides cells expressing a fusion protein of the present disclosure or comprising a polynucleotide or vector encoding the fusion protein. The cells can be stem cells, progenitor cells, and/or immune cells modified to express a fusion protein described herein. In some embodiments, a cell line derived from an immune cell is used. Non-limiting examples of cells, as provided herein, include mesenchymal stem cells (MSCs), natural killer (NK) cells, NKT cells, innate lymphoid cells, mast cells, eosinophils, basophils, macrophages, neutrophils, mesenchymal stem cells, dendritic cells, T cells (e.g., CD8+ T cells, CD4+ T cells, gamma-delta T cells, and T regulatory cells (CD4+, FOXP3+, CD25+)) and B cells. In some embodiments, the cell a stem cell, such as pluripotent stem cell, embryonic stem cell, adult stein cell, bone-marrow stem cell, umbilical cord stein cells, or other stem cell.

The cells can be modified to express a fusion protein provided herein. In some embodiment, the fusion protein comprises an inducible receptor. The inducible receptor can comprise a single chain receptor (i.e., a single fusion protein) or a multichain receptor (i.e., multiple fusion proteins). When the inducible cell receptor is a multichain receptor, the cells comprise multiple fusion proteins. Accordingly, the present disclosure provides a cell (e.g., a population of cells) engineered to express an inducible receptor, such as a chimeric antigen receptor (CAR), wherein the receptor comprises an antigen-binding domain, a transmembrane domain, and an intracellular signaling domain.

### Pharmaceutical Compositions

Pharmaceutical compositions of the present disclosure can comprise a fusion protein or a cell expressing the fusion protein (e.g., a plurality of fusion protein-expressing cells), as described herein, in combination with one or more pharmaceutically or physiologically acceptable carriers, diluents or excipients. Such compositions can comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

Pharmaceutical compositions of the present disclosure can be administered in a manner appropriate to the disease to be treated (or prevented). The quantity and frequency of administration can be determined by such factors as the condition of the patient, and the type and severity of the patient's disease, although appropriate dosages may be determined by clinical trials.

In preferred embodiments, the pharmaceutical composition is substantially free of a contaminant, such as endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. The pharmaceutical composition can be free from bacterium such as Alcaligenes faecalis, Candida albicans, Escherichia coli, Haemophilus influenza, Neisseria meningitides, Pseudomonas aeruginosa, Staphylococcus aureus, Streptococcus pneumonia, and Streptococcus pyogenes group A.

### Method of Preparing Therapeutic Cells

In one aspect, the present disclosure provides a method of preparing a modified cell comprising a fusion protein for experimental or therapeutic use.

Ex vivo procedures for making therapeutic fusion protein-modified cells are well known in the art. For example, cells are isolated from a mammal (e.g, a human) and genetically modified (i.e., transduced or transfected in vitro) with a vector expressing a fusion protein disclosed herein. The fusion protein-modified cell can be administered to a mammalian recipient to provide a therapeutic benefit. The mammalian recipient may be a human and the fusion protein-modified cell can be autologous with respect to the recipient. Alternatively, the cells can be allogeneic, syngeneic or xenogeneic with respect to the recipient. The procedure for ex vivo expansion of hematopoietic stem and progenitor cells is described in U.S. Pat. No. 5,199,942, incorporated herein by reference, can be applied to the cells of the present disclosure. Other suitable methods are known in the art, therefore the present disclosure is not limited to any particular method of ex vivo expansion of the cells.

### Method of Use

In one aspect, the present disclosure provides a type of cell therapy where a population of cells is genetically modified to express a fusion protein provided herein and the modified cells are administered to a subject in need thereof. In some embodiments, the methods comprise culturing the population of cells (e.g. in cell culture media) to a desired cell density (e.g., a cell density sufficient for a particular cell-based therapy). In some embodiments, the population of cells are cultured in the absence of a protease inhibitor that represses activity of the protease or in the presence of a protease inhibitor that represses activity of the protease.

In another aspect, the present disclosure provides a type of therapy where a pharmaceutical composition comprising a fusion protein provided herein is administered to a subject in need thereof.

In some embodiments, the method comprises administering a protease inhibitor that represses activity of the protease after administration of the modified cells or the pharmaceutical composition. In some embodiments, the method further comprises withdrawing the protease inhibitor after administration of the modified cells or the pharmaceutical composition.

In some embodiments, administration of the protease inhibitor to a subject induces degradation of the polypeptide of interest. In some embodiments, administration of the protease inhibitor protects the polypeptide of interest from degradation. In some embodiments, withdrawal of the protease inhibitor from a subject induces degradation of the polypeptide of interest. In some embodiments, withdrawing the protease inhibitor from a subject protects the polypeptide of interest from degradation.

In some embodiments, administration of the protease inhibitor to a subject induces activation of the polypeptide of interest. In some embodiments, administration of the protease inhibitor induces inhibition of the polypeptide of interest. In some embodiments, withdrawing the protease inhibitor from a subject induces activation of the polypeptide of interest. In some embodiments, withdrawing the protease inhibitor from a subject induces inhibition of the polypeptide of interest.

In some embodiments, the population of cells are cultured in the presence of a protease inhibitor that represses activity of the protease to degrade the polypeptide of interest to produce an expanded population of cells. For example, in some embodiments the fusion protein comprises a positioned at the C-terminal end of the polypeptide of interest such that when the cells are cultured in the presence of the protease inhibitor, the protease is inactivated and unable to cleave the cognate cleavage site that separates, for example, the C-terminal end of the polypeptide of interest from the degron. Thus, the degron remains fused to the polypeptide of interest and promotes degradation of the polypeptide through either the proteasome or an autophagy-lysosome pathway. This is particularly advantageous, for example, if the polypeptide of interest is a product that is toxic to the cells or inhibits cell survival and/or proliferation/expansion of the cells.

In some embodiments, the population of cells is cultured for a period of time that results in the production of an expanded cell population that comprises at least 2-fold the number of cells of the starting population. In some embodiments, the population of cells is cultured for a period of time that results in the production of an expanded cell population that comprises at least 4-fold the number of cells of the starting population. In some embodiments, the population of cells is cultured for a period of time that results in the production of an expanded cell population that comprises at least 16-fold the number of cells of the starting population.

In some embodiments, the methods further comprise withdrawing the protease inhibitor from the expanded population of cells. The protease inhibitor may be removed, for example, by simply washing the cells with fresh culture media. In the absence of the protease inhibitor, the cells are able to produce the polypeptide of interest, e.g., in vivo following administration of the cells to a subject in need.

Thus, in some embodiments, the methods comprise delivering cells of the expanded population of cells to a subject in need of a cell-based therapy. In some embodiments, the subject is a human subject. In some embodiments, the subject in need has an autoimmune condition. In some embodiments, the subject in need has a cancer (e.g., a primary cancer or a metastatic cancer).

Thus, in some embodiments, the polypeptide of interest encodes a therapeutic protein. Examples of therapeutic proteins include, but are not limited to, T cell receptors (TCRs), chimeric T cell receptors, artificial T cell receptors, synthetic T cell receptors, chimeric immunoreceptors, antibody-coupled T cell receptors (ACTRs), T cell receptor fusion constructs (TRUCs), chimeric antigen receptors (CARs), antibodies. Fc fusion proteins, anticoagulants, blood factors, bone morphogenetic proteins, engineered protein scaffolds, enzymes, growth factors, hormones, interferons, interleukins, and thrombolytics.

The methods, in some embodiments, may comprise administering to the subject a protease inhibitor that represses activity of the protease to degrade the polypeptide of interest. The protease inhibitor may be administered any time following administration of the cell-based therapy (the expanded cells containing the polypeptide of interest) In some embodiments, the protease inhibitor is administered 1 week, 2 weeks, 3 weeks, 1 month, 2 months, 3 months, 6 months, 9 months, 1 year, 2 years, 3 years, 4 years, or 5 years after the subject has received the cell-based therapy. In some embodiments, the protease inhibitor is administered depending on the health condition of the subject.

Also provided herein are methods of regulating activity of a protein of interest either in vivo or ex vivo. In some embodiments, the activity of the protein of interest is regulated in Pivo by delivering to a subject in need of a cell-based therapy a population of cells that comprise a polynucleotide that encodes a fusion protein of the present disclosure comprising the protein of interest fused to a sequence encoding a degron, and administering to the subject a protease inhibitor that represses activity of the protease to degrade the protein of interest. In some embodiments, the protein of interest is a therapeutic protein. In some embodiments, the method can comprise the step of withdrawing a protease inhibitor that represses activity of the protease from a subject. The protease inhibitor may be withdrawn any time following administration of the cell-based therapy (the expanded cells containing the gene of interest). In some embodiments, the protease inhibitor is withdrawn 1 week, 2 weeks, 3 weeks, 1 month, 2 months, 3 months, 6 months, 9 months, 1 year, 2 years, 3 years, 4 years, or 5 years after the subject has received the cell-based therapy. In some embodiments, the protease inhibitor is withdrawn for 1 week, 2 weeks, 3 weeks, 1 month, 2 months, 3 months, 6 months, 9 months, 1 year, 2 years, 3 years, 4 years, or 5 years. In some embodiments, the protease inhibitor is withdrawn depending on the health condition of the subject.

In some embodiments, the activity of the protein of interest is regulated by providing a population of cells comprising a fusion protein of the present disclosure or a polynucleotide encoding the fusion protein and contacting the population of cells with a protease inhibitor that represses activity of the protease. In some embodiments, the method further comprises removing the protease inhibitor from the population of cells. The protease inhibitor may be removed any time following contacting of the population of cells with the protease inhibitor. In some embodiments, the protease inhibitor is removed 1 week, 2 weeks, 3 weeks, 1 month, 2 months, 3 months, 6 months, 9 months, 1 year, 2 years, 3 years, 4 years, or 5 years after following contacting of the population of cells with the protease inhibitor. In some embodiments, the protease inhibitor is removed for 1 week, 2 weeks, 3 weeks, 1 month, 2 months, 3 months, 6 months, 9 months, 1 year, 2 years, 3 years, 4 years, or 5 years. In some embodiments, the population of cells is administered to a subject in need of a cell-based therapy.

### Kits

Fusion proteins or nucleic acids encoding them as well as conditionally replicating viral vectors can be provided in kits with suitable instructions and other necessary reagents for preparing or using them, as described above. The kit may contain in separate containers fusion proteins, and/or recombinant constructs for producing fusion proteins, and/or conditionally replicating viral vectors, and/or cells (either already transfected or separate). Additionally, instructions (e.g., written, tape, VCR, CD-ROM, DVD, Blu-ray, flash drive, etc.) for using the fusion proteins or viral vectors may be included in the kit. The kit may further include a protease inhibitor, such as an HCV NS3 protease inhibitor, including, for example, simeprevir, danoprevir, asunaprevir, ciluprevir, boceprevir, sovaprevir, paritaprevir, telaprevir, grazoprevir, glecaprevir, or voxiloprevir. The kit may also contain other packaged reagents and materials (e.g., transfection reagents, buffers, media, and the like).

### EXAMPLES

### Example 1: Single and Double Deimmunized Variants of NS3 Protease/NS4 Degron Fusion Protein

Four different fusion proteins containing the following were generated: 1) chimeric antigen receptor (CAR) polypeptide of interest, 2) variant HCV NS3 protease, 3) cognate protease cleavage site, and 4) HCV NS4 degron operably linked to the CAR polypeptide of interest. The four different fusion proteins differ from one another based on one or more mutations in the variant HCV NS3 protease. The mutations of the different fusion proteins were tested to determine whether they could reduce immunogenicity while maintaining protease activity, thereby ensuring controllability over the CAR. Specifically, the four fusion proteins have the following mutations in the variant HCV NS3 protease.

Fusion Protein 1 (T1080A): Includes a Thr to Ala substitution at a position corresponding to position 1080 of the sequence shown in SEQ ID NO: 1.

Fusion Protein 2 (T1080A, V1077A): Includes a Thr to Ala substitution at a position corresponding to position 1080 of the sequence shown in SEQ ID NO: 1 and a Val to Ala substitution at a position corresponding to position 1077 of the sequence shown in SEQ ID NO: 1.

Fusion Protein 3 (T1080A, W1079A). Includes a Thr to Ala substitution at a position corresponding to position 1080 of the sequence shown in SEQ ID NO: 1 and a Trp to Ala substitution at a position corresponding to position 1079 of the sequence shown in SEQ ID NO: 1.

Fusion Protein 4 (T1080A, V1081A) Includes a Thr to Ala substitution at a position corresponding to position 1080 of the sequence shown in SEQ ID NO: 1 and a Val to Ala substitution at a position corresponding to position 1081 of the sequence shown in SEQ ID NO: 1.

On Day 0, total pan T-cell populations were isolated from peripheral blood mononuclear cells (PBMCs) and stimulated using Dynabeads®. On Day 1, T-cell populations underwent lentiviral transduction. On Day 2, cell media was changed to remove lentivirus and LentiBlast media. On Day 4, Dynabeads® were removed. On Day 7, cell media was changed and the T-cells were treated. To test the controllability of the CAR polypeptide of the fusion proteins, a first population was treated with 2 μM asunaprevir, a small molecule inhibitor of hepatitis C whereas a second population was left unreated (No ASV). On Day 9, flow cytometry using YFP and myc-tag (Alexa647) fluorescent tags was performed on the ASV treated T-cells and the non-ASV treated T-cells to determine the level of CAR expression in the cells.

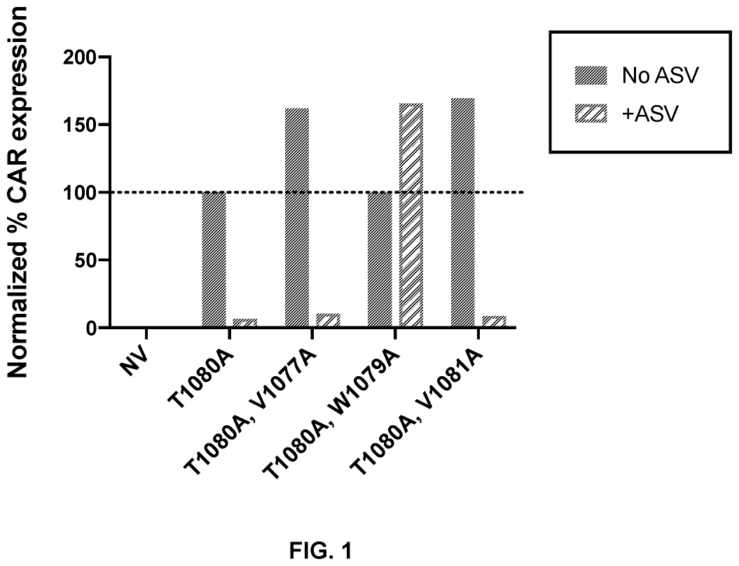

FIG. 1 depicts the normalized % CAR expression in cells transfected to express one of the four different fusion proteins. The cells were either treated with asunaprevir (+ASV) or untreated (No ASV). Each of the values were normalized to the CAR expression in T1080A variant expressing cells that were untreated. Notably, Fusion Protein 2 (T1080A, V1077A), Fusion Protein 3 (T1080A, W1079A), and Fusion Protein 4 (T1080A, V1081A) exhibited close to or higher levels (e.g., 100%-150%) of CAR expression in comparison to Fusion Protein 1 (T1080A), thereby indicating that the additional mutations do not compromise the degron functionality. For three of the fusion proteins, Fusion Protein 1 (T1080A), Fusion Protein 2 (T1080A, V1077A), and Fusion Protein 4 (T1080A, V1081A), asunaprevir treatment significantly reduced the relative percentage of CAR expression. This indicates that the inhibition of the HCV NS3 protease by the asunaprevir directly led to the reduced CAR expression levels. Altogether, these results demonstrate the controllability of the expression of a polypeptide of interest, such as a CAR, on deimmunized fusion proteins by using small molecule knockdowns (e.g., using asunaprevir).

SEQUENCES

| SEQ ID | | |
|---|---|---|
| NO | Identity | Sequence |
| SEQ ID | HCV 1a | 10 20 30 40 50 |
| NO: 1 | polyprotein | MSTNPKPQKK NKRNTNRRPQ DVKFPGGGQI VGGVYLLPRR GPRLGVRATR |
| | | 60 70 80 90 100 |
| | | KTSERSQPRG RRQPIPKARR PEGRTWAQPG YPWPLYGNEG CGWAGWLLSP |
| | | 110 120 130 140 150 |
| | | RGSRPSWGPT DPRRRSRNLG KVIDTLTCGF ADLMGYIPLV GAPLGGAARA |
| | | 160 170 180 190 200 |
| | | LAHGVRVLED GVNYATGNLP GCSFSIFLLA LLSCLTVPAS AYQVRNSTGL |
| | | 210 220 230 240 250 |
| | | YKVTNDCPNS SIVYEAADAI LHTPGCVPCV REGNASRCWV AMTPTVATRD |
| | | 260 270 280 290 300 |
| | | GKLPATQLRR HIDLLVGSAT LCSALYVGDL CGSVFLVGQL FTFSPRRHWT |
| | | 310 320 330 340 350 |
| | | TQGCNCSIYP GHITGHRMAW DMMMNWSPTT ALVMAQLLRI PQAILDMIAG |
| | | 360 370 380 390 400 |
| | | AHWGVLAGIA YPSMVGNWAK VLVVLLLFAG VDAETHVTGG SAGHTVSGFV |
| | | 410 420 430 440 450 |
| | | SLLAPGAKQN VQLINTNGSW HLNSTALNCN DSLNTGWLAG LFYHHKFNSS |
| | | 460 470 480 490 500 |
| | | GCPERLASCR PLTDFDQGWG PISYANGSGP DQRPYCWHYP PKPCGIVPAK |
| | | 510 520 530 540 550 |
| | | SVCGPVYCFT PSPWVGTTD RSGAPTYSWG SNDTDVFVLN NTRPPLGNVVF |
| | | 560 570 580 590 600 |
| | | GCTWMNSTGF TKVCGAPPCV IGGAGNNTLH CPTDCFRKHP DATYSRCGSG |
| | | 610 620 630 640 650 |
| | | PWITPRCLVD YPYRLWHYPC TINYTIFKIR MYVGGVEHRL EAACNWTRGE |
| | | 660 670 680 690 700 |
| | | RCDLEDRDRS ELSPLLLTTT QWQVLPCSFT TLPALSTGLI HLHQNIVDVQ |
| | | 710 720 730 740 750 |
| | | YLYGVGSSIA SWAIKWEYVV LLFLLLADAR VCSCLWMMLL ISQAEAALEN |
| | | 760 770 780 790 800 |
| | | LVILNAASLA GTKGLVSFLV FFCFAWYLKG KWVPGAVYTF YGMWPLLLLL |
| | | 810 820 830 840 850 |
| | | LALPQRAYAL DTEVAASCGG WLVGLMALT LSPYYKRYIS WCLWWLQYFL |
| | | 860 870 880 890 900 |
| | | TRVEAQLHVW IPPLNVRGGR DAVILLMCAV HPTLVFDITK LLLAVFGPLW |
| | | 910 920 930 940 950 |
| | | ILQASLLKVP YFVRVQGLLR FCALARKMIG GHYVQMVIIK LGALTGTYVY |
| | | 960 970 980 990 1000 |
| | | NKLTPLRDWA HNGLRDLAVA VEPVVFSQME TKLITWGADT AACGDIINGL |
| | | 1010 1020 1030 1040 1050 |
| | | PVSARRGREI LLGPADGMVS KGWRLLAPIT AYAQQTRGLL GCIITSLTGR |
| | | 1060 1070 1080 1090 1100 |
| | | DKNQVEGEVQ IVSTAAQTFL ATCINGVCWT VYHGAGTRTI ASPKGPVIQM |
| | | 1110 1120 1130 1140 1150 |
| | | YTNVDQDLVG WPAPQGSRSL TPCTCGSSDL YLVTRHADVI PVRRRGDSRG |
| | | 1160 1170 1180 1190 1200 |
| | | SLLSPRPISY LKGSSGGPLL CPAGHAVGIF RAAVCTRGVA KAVDFIPVEN |
| | | 1210 1220 1230 1240 1250 |
| | | LETTMRSPVP TDNSSPPVVP QSFQVAHLHA PTGSGKSTKV PAAYAAQGYK |
| | | 1260 1270 1280 1290 1300 |
| | | VLVLNPSVAA TLGFGAYMSK AHGIDPNIRT GVRTITTGSP ITYSTYGKFL |
| | | 1310 1320 1330 1340 1350 |
| | | ADGGCSGGAY DIIICDECHS TDATSILGIG TVLDQAETAG ARLVVLATAT |
| | | 1360 1370 1380 1390 1400 |
| | | PPGSVTVPHP NIEEVALSTT GEIPFYGKAI PLEVIKGGRH LIFCKSKKKC |
| | | 1410 1420 1430 1440 1450 |
| | | DELAAKLVAL GINAVAYYRG LDVSVIPTSG DVVVVATDAL MTGYTGDFDS |
| | | 1460 1470 1480 1490 1500 |
| | | VIDCNTCVTQ TVDFSLDPTF TIETITLPQD AVSRTQRRGR TGRGKPGIYR |
| | | 1510 1520 1530 1540 1550 |
| | | FVAPGERPSG MFDSSVLCEC YDAGCAWYEL TPAETTVRLR AYMNTPGLPV |
| | | 1560 1570 1580 1590 1600 |
| | | CQDHLEFWEG VFTGLTHIDA HFLSQTKQSG ENLPYLVAYQ ATVCARAQAP |
| | | 1610 1620 1630 1640 1650 |
| | | PPSWDQMWKC LIRLKPTLHG PTPLLYRLGA VQNEITLTHP VTKYIMTCMS |
| | | 1660 1670 1680 1690 1700 |
| | | ADLEVVTSTW VLVGGVLAAL AAYCLSTGCV VIVGRVVLSG KPAIIPDREV |
| | | 1710 1720 1730 1740 1750 |
| | | LYREFDEMEE CSQHLPYIEQ GMMLAEQFKQ KALGLLQTAS RQAEVIAPAV |
| | | 1760 1770 1780 1790 1800 |
| | | QTNWQKLETF WAKHMWNFIS GIQYLAGLST LPGNPAIASL MAFTAAVTSP |
| | | 1810 1820 1830 1840 1850 |
| | | LTTSQTLLFN ILGGWVAAQL AAPGAATAFV GAGLAGAAIG SVGLGKVLID |
| | | 1860 1870 1880 1890 1900 |
| | | ILAGYGAGVA GALVAFKIMS GEVPSTEDLV NLLPAILSPG ALVVGVVCAA |
| | | 1910 1920 1930 1940 1950 |
| | | ILRRHVGPGE GAVQWMNRLI AFASRGNHVS PTHYVPESDA AARVTAILSS |
| | | 1960 1970 1980 1390 2000 |
| | | LTVTQLLRRL HQWISSECTT PCSGSWLRDI WDWICEVLSD FKTWLKAKLM |
| | | 2010 2020 2030 2040 2050 |
| | | PQLPGIPFVS CQRGYKGVWR VDGIMHTRCH CGAEITGKVK NGTMRIVGPR |
| | | 2060 2070 2080 2090 2100 |
| | | TCRNMWSGTF PINAYTTGPC TPLPAPNYTF ALWRVSAEEY VEIRQVGDFH |
| | | 2110 2120 2130 2140 2150 |
| | | YVTGMTTDNL KCPCQVPSPE FFTELDGVRL HRFAPPCKPL LREEVSFRVG |
| | | 2160 2170 2180 2190 2200 |
| | | LKEYPVGSQL PCEPEPDVAV LTSMLTDPSH ITAEAAGRRL ARGSPPSVAS |
| | | 2210 2220 2230 2240 2250 |
| | | SSASQLSAPS LKATCTANHD SPDAELIEAN LLWRQEMGGN ITRVESENKV |
| | | 2260 2270 2280 2290 2300 |
| | | VILDSFDPLV AEEDEREISV PAEILRKSRR FAQALPVWAR PDYNPPLVET |
| | | 2310 2320 2330 2340 2350 |
| | | WKKPDYEPPV VKGCPLPPPK SPPVPPPRKK RTVVLTESTL STALAELATR |
| | | 2360 2370 2380 239O 2400 |
| | | SFGSSSTSGI TGDNTTTSSE PAPSGCPPDS DAESYSSMPP LEGEPGDPDL |
| | | 2410 2420 2430 2440 2450 |
| | | SDGSWSTVSS EANAEDWCC SMSYSVVTGAL VTPCAAEEQK LPINALSNSL |
| | | 2460 2470 2480 2490 2500 |
| | | LRHHNLVYST TSRSACQRQK KVTFDRLQVL DSHYQDVLKE VKAAASKVKA |
| | | 2510 2520 2530 2540 2550 |
| | | NLLSVEEACS LTPPHSAKSK FGYGAKDVRC HARKAVTHIN SVWKDLLEDN |
| | | 2560 2570 2580 2590 2600 |
| | | VTPIDTTIMA KNEVFCVQPE KGGRKPARLI VFPDLGVRVC EKMALYDVVT |
| | | 2610 2620 2630 2640 2650 |
| | | KLPLAVMGSS YGFQYSPGQR VEFLVQAWKS KKTPMGFSYD TRCFDSTVTE |
| | | 2660 2670 2680 2690 2700 |
| | | SDIRTEEAIY QCCDLDPQAR VAIKSLTERL YVGGPLTNSR GENCGYRRCR |
| | | 2710 2720 2730 2740 2750 |
| | | ASGVLTTSCG NTLTCYIKAR AACRAAGLQD CTMLVCGDDL VVICESAGVQ |
| | | 2760 2770 2780 2790 2800 |
| | | EDAASLRAFT EAMTRYSAPP GDPPQPEYBL SLITSCSSNV SVAHDGAGKR |
| | | 2810 2820 2830 2840 2850 |
| | | VYYLTRDPTT PLARAAWETA RHTPVNSWLG NIIMFAPTLW ARMILMTHFF |
| | | 2860 2870 2880 2890 2900 |
| | | SVLIARDQLB QALDCEIYGA CYSIEPLDLP PIIQRLHGLS AFSLHSYSPG |
| | | 2910 2920 2930 2940 2950 |
| | | EINRVAACLR KLGVPPLRAW RHRARSVRAR LLARGGRAAI CGKYLFNWAV |
| | | 2960 2970 2980 2990 3000 |
| | | RTKLKLTPIA AAGQLDLSGW FTAGYSGGDI YHSVSHARPS WIWFCLLLLA |
| | | 3010 |
| | | AGVGIYLLPN R |
| SEQ ID | a variant NS3 | APITAYAQQT RGLLGCIITS LTGRDKNQVE GEVQIVSTAT QTFLATCING |
| NO: 2 | protease is | VCWAVYHGAG TRTIASPKGP VIQMYTNVDQ DLVGWPAPQG |
| | derived from | SRSLTPCTCG SSDLYLVTRH ADVIPVRRRG DSRGSLLSPR PISYLKGSSG |
| | an HCV NS3 | GPLLCPAGHA VGLFRAAVCT RGVAKAVDFI PVENLETTMR SPVFTD |
| SEQ ID | HCV NS4A | TWVLVGGVLA ALAAYCLSTG CWIVGRIVL SGKPAIIPDR EVLY |
| NO: 3 | co-factor | |
| SEQ ID | cognate | CMSADLEVVTSTWVLVGGVL |
| NO: 4 | protease | |
| | cleavage site | |
| SEQ ID | cognate | YQEFDEMEECSQHLPYIEQG |
| NO: 5 | protease | |
| | cleavage site | |
| SEQ ID | cognate | WISSECTTPCSGSWLRDIWD |
| NO: 6 | protease | |
| | cleavage site | |
| SEQ ID | cognate | GADTEDVVCCSMSYSWTGAL |
| NO: 7 | protease | |
| | cleavage site | |
| SEQ ID | cognate | ADLEVVTSTWL |
| NO: 8 | protease | |
| | cleavage site | |
| SEQ ID | cognate | DEMEECSQHL |
| NO: 9 | protease | |
| | cleavage site | |
| SEQ ID | cognate | ECTTPCSGSWL |
| NO: 10 | protease | |
| | cleavage site | |
| SEQ ID | cognate | EDVVPCSMG |
| NO: 11 | protease | |
| | cleavage site | |
| SEQ ID | HCV NS3 | APITAYAQQT RGLLGCIITS LTGRDKNQVE GEVQIVSTAA QTFLATCING |
| NO: 12 | protease | VCWTVYHGAG TRTIASSKGP VIQMYTNVDQ DLVGWPAPQG |
| | | ARSLTPCTCG SSDLYLVTRH ADVIPVRRRG DGRGSLLSPR PISYLKGSSG |
| | | GPLLCPAGHA VGIFRAAVCT RGVAKAVDFI PVEGLETTMR SPVFSD |
| SEQ ID | HIV-I | PQVTLWQRPLVTIKIGGQLKEALLDTGADDTVLEEMSLPGRWKPKMIGGIGG |
| NO: 13 | protease | FIKVRQYDQILIEICGHKAIGTVLVGPTPVNIIGRNLLTQIGCTLNF |
| SEQ ID | fluorogenic | EDANS-EPLFAERK-DABCYL |
| NO: 14 | calpain | |
| | substrate | |
| SEQ ID | Caspasc 1 | YVAD |
| NO: 15 | cleavage sile | |
| SEQ ID | Caspase 2 | VDVAD |
| NO: 16 | cleavage site | |
| SEQ ID | Caspase 4 | DEVD |
| NO: 17 | cleavage site | |
| SEQ ID | Caspase 6 | VEHD |
| NO: 18 | cleavage sile | |
| SEQ ID | Caspasc 9 | LGHD |
| NO: 19 | cleavage site | |
| SEQ ID | Caspasc 10 | LQTDG |
| NO: 20 | cleavage site | |
| SEQ ID | angiotensin | MGAASGRRGP GLLLPLPLLL LLPPQPALAL DPGLQPGNFS ADEAGAQLFA |
| NO: 21 | converting | QSYNSSAEQV LFQSVAASWA HDTNITAENA RRQEEAALLS QEFAEAWGQK |
| | enzyme | AKELYEPIWQ NFTDPQLRRI IGAVRTLGSA NLPLAKRQQY NALLSNMSRI |
| | (ACE) | YSTAKVCLPN KTATCWSLDP DLTNILASSR SYAMLLFAWE GWHNAAGIPL |
| | | KPLYEDFTAL SNEAYKQDGF TDTGAYWRSW YNSPTFEDDL EHLYQQLEPL |
| | | YLNLHAFVRR ALHRRYGDRY INLRGPIPAH LLGDMWAQSW ENIYDMVVPF |
| | | PDKPNLDVTS TMLQQGWNAT HMFRVAEEFF TSLELSPMPP EFWEGSMLEK |
| | | PADGREVVCH ASAWDFYNRK DFRIKQCTRV TMDQLSTVHH EMGHIQYYLQ |
| | | YKDLPVSLRR GANPGFHEAI GDYLALSVST PEHLHKIGLL DRVTNDTESD |
| | | INYLLKMALE KIAFLPFGYL VDQWRWGVFS GRTPPSRYNF DWWYLRTKYQ |
| | | GICPPVTRNE THFDAGAKFH VPNVTPYIRY FVSFVLQFQF HEALCKEAGY |
| | | EGPLHQCDIY RSTKAGAKLR KVLQAGSSRP WQEVLKDMVG LDALDAQPLL |
| | | KYFQPVTQWL QEQNQQNGEV LGWPEYQWHP PLPDNYPEGI DLVTDEAEAS |
| | | KFVEEYDRTS QVVWNEYAEA NWNYNTNITT ETSKILLQKN MQ1ANHTLKY |
| | | GTQARKFDVN QLQNTTIKRI IKKVQDLERA ALPAQELEEY NKILLDMETT |
| | | YSVATVGHPN GSCLQLEPDL TNVMATSRKY EDLLWAWEGW RDKAGRAILQ |
| | | FYPKYVELIN QAARLNGYVD AGDSWRSMYE TPSLEQDLER LFQELQPLYL |
| | | NLHAYVRRAL HRHYGAQHIN LEGPIPAHLL GNMWAQTWSN IYDLVVTFPS |
| | | APSMDTTEAM LKQGWTPRRM FKEADDFFTS LGLLPVPPEF WNKSMLEKPT |
| | | DGREVVCHAS AWDFYNGKDF RIKQCTTVNL EDLVVAHHEM GHIQYFMQYK |
| | | DLPVALREGA NPGFHEAIGD VLALSVSTPK HLHSLNLLSS EGGSDEHDIN |
| | | FLMKMALDKI AFIPFSYLVD QWRWRVFDGS iTKENYNQEW WSLRLKYQGL |
| | | CPPVPRTQGD FDPGAKFHIP SSVPYIRYFV SFIIQFQFHE ALCQAAGHTG |
| | | PLHKCDIYQS KEAGQRLATA MKLGFSRPWP EAMQLITGQP NMSASAMLSY |
| | | FKPLLDWLRT ENELHGEKLG WPQYNWTPNS ARSEGPLPDS GRVSFLGLDL |
| | | DAQQARVGQW LLLFLGIALL VATLGLSQRL FSIRHRSLHR HSHGPQFGSE |
| | | VELRHS |
| SEQ ID | amyloid | EVNLDAEF |
| NO: 22 | precursor | |
| | protein | |
| | secretase beta | |
| | cleavage site | |
| SEQ ID | MMP2 | PQGIAGQ |
| NO: 23 | cleavage sile | |
| SEQ ID | tobacco Etch | ENLYFQS |
| NO: 24 | virus (TEV) | |
| | protease | |
| | cleavage site | |
| SEQ ID | Cleavage site | HPFHL |
| NO: 25 | | |
| SEQ ID | DENV | SGVLWDTPSPPEVERAVLDDGIYRIMQRGLLGRSQ |
| NO: 26 | NS3pro | VGVGVFQDGVFHTMWHVTRGAVLMYQGKRLEPSWA |
| | (NS2B/NS3) | SVKKDLISYGGGWRFQGSWNTGEEVQVIAVEPGKN |
| | | PKNVQTAPGTFKTPEGEVGAIALDFKPGTSGSPIV |
| | | NREGKIVGLYGNGVVTTSGTYVSAIAQAKASQEGP |
| | | LPEIEDEVFRKRNLTIMDLHPGSGKTRRYLPAIVR |
| | | EAIRRNVRTLILAPTRVVASEMAEALKGMPIRYQT |
| | | TAVKSEHTGKEIVDLMCHATFTMRLLSPVRVPNYN |
| | | MIIMDEAHFTDPASIARRGYISTRVGMGEAAAIFM |
| | | TATPPGSVEAFPQSNAVIQDEERDIPERSWNSGYE |
| | | WITDFPGKTVWFVPSIKSGNDIANCLRKNGKRVIQ |
| | | LSRKTFDTEYQKTKNNDWDYVVTTDISEMGANFRA |
| | | DRVIDPRRCLKPVILKDGPERVILAGPMPVTVASA |
| | | AQRRGRIGRNQNKEGDQYVYMGQPLNNDEDHAHWT |
| | | EAKMLLDNINTPEGIIPALFEPEREKSAAIDGEYR |
| | | LRGEARKTFVELMRRGDLPVWLSYKVASEGFQYSD |
| | | RRWCFDGERNNQVLEENMDVEMWTKEGERKKLRPR |
| | | WLDARTYSDPLALREFKEFAAGRR |
| SEQ ID | DENV | AGVLWDVPSPPPVGKAELEDGAYRIKQKGILGYSQ |
| NO: 27 | NS3pro | IGAGVYKEGTFHTMWHVTRGAVLMHKGKRIEPSWA |
| | (NS2B/NS3) | DVKKDLISYGGGWKLEGEWKEGEEVQVLALEPGKN |
| | | PRAVQTKPGLFKTNAGTIGAVSLDFSPGTSGSPII |
| | | DKKGKWGLYGNGVVTRSGAYVSAIAQTEKSIEDNP |
| | | EIEDDIFRKRKLTIMDLHPGAGKTKRYLPAIVREA |
| | | IKRGLRTLILAPTRWAAEMEEALRGLPIRYQTPAI |
| | | RAEHTGREIVDLMCHATFTMRLLSPVRVPNYNLII |
| | | MDEAHFTDPASIAARGYISTRVEMGEAAGIFMTAT |
| | | PPGSRDPFPQSNAPIMDEEREIPERSWSSGHEWVT |
| | | DFKGKTVWFVPSIKAGNDIAACLRKNGKKVIQLSR |
| | | KTFDSEYVKTRTNDWDFWTTDISEMGANFKAERVI |
| | | DPRRCMKPVILTDGEERVILAGPMPVTHSSAAQRR |
| | | GRIGRNPKNENDQYIYMGEPLENDEDCAHWKEAKM |
| | | LLDNINTPEGIIPSMFEPEREKVDAIDGEYRLRGE |
| | | ARKTFVDLMRRGDLPVWLAYRVAAEGINYADRRWC |
| | | FDGIKNNQILEENVEVEIWTKEGERKKLKPRWLDA |
| | | KIYSDPLALKEFKEFAAGRK |
| SEQ ID | DENV | SGVLWDVPSPPETQKAELEEGVYRIKQQGIFGKTQ |
| NO: 28 | NS3pro | VGVGVQKEGVFHTMWHVTRGAVLTHNGKRLEPNWA |
| | (NS2B/NS3) | SVKKDLISYGGGWRLSAQWQKGEEVQVIAVEPGKN |
| | | PKNFQTMPGIFQTTTGEIGAIALDFKPGTSGSPII |
| | | NREGKWGLYGNGVVTKNGGYVSGIAQTNAEPDGPT |
| | | PELEEEMFKKRNLTIMDLHPGSGKTRKYLPAIVRE |
| | | AIKRRLRTLILAPTRVVAAEMEEALKGLPIRYQTT |
| | | ATKSEHTGREIVDLMCHATFTMRLLSPVRVPNYNL |
| | | IIMDEAHFTDPASIAARGYISTRVGMGEAAAIFMT |
| | | ATPPGTADAFPQSNAPIQDEERDIPERSWNSGNEW |
| | | ITDFVGKTVWFVPSIKAGNDIANCLRKNGKKVIQL |
| | | SRKTFDTEYQKTKLNDWDFWTTDISEMGANFKADR |
| | | VIDPRRCLKPVTLTDGPERVILAGPMPVTVASAAQ |
| | | RRGRVGRNPQKENDQYIFMGQPLNKDEDHAHWTEA |
| | | KMLLDNINTPEGIIPALFEPEREKSAAIDGEYRLK |
| | | GESRKTFVELMRRGDLPVWLAHKVASEGIKYTDRK |
| | | WCFDGERNNQILEENMDVEIWTKEGEKKKLRPRWL |
| | | DARTYSDPLALKEFKDFAAGRK |
| SEQ ID | DENV | SGALWDVPSPAATQKAALSEGVYRIMQRGLFGKTQ |
| NO: 29 | NS3pro | VGVGIHIEGVFHTMWHVTRGSVICHETGRLEPSWA |
| | (NS2B/NS3) | DVRNDMISYGGGWRLGDKWDKEEDVQVLAIEPGKN |
| | | PKHVQTKPGLFKTLTGEIGAVTLDFKPGTSGSPI |
| | | INRKGKVIGLYGNGVVTKSGDYVSAITQAERIGEP |
| | | DYEVDEDIFRKKRLTIMDLHPGAGKTKRILPSIVR |
| | | EALKRRLRTLILAPTRVVAAEMEEALRGLPIRYQT |
| | | PAVKSEHTGREIVDLMCHATFTTRLLSSTRVPNYN |
| | | LIVMDEAHFTDPSSVAARGYISTRVEMGEAAAIFM |
| | | TATPPGTTDPFPQSNSPIEDIEREIPERSWNTGFD |
| | | WITDYQGKTVWFVPSIKAGNDIANCLRKSGKKVIQ |
| | | LSRKTFDTEYPKTKLTDWDFVVTTDISEMGANFRA |
| | | GRVTDPRRCLKPVILPDGPERVTLAGPIPVTPASA |
| | | AQRRGRIGRNPAQEDDQYVFSGDPLKNDEDHAHWT |
| | | EAKMLLDNIYTPEGIIPTLFGPEREKTQAIDGEFR |
| | | LRGEQRKTFVELMRRGDLPVWLSYKVASAGISYKD |
| | | REWCFTGERNNQILEENMEVEIWTREGEKKKLRPK |
| | | WLDARVYADPMALKDFKEFASGRK |
| SEQ ID | Sub-sequence | GLLGCIITSL |
| NO: 30 | of HCV 1a | |
| | polyprotein | |
| SEQ ID | Sub-sequence | GEVQIVSTAAQTFLATCINGVCWTVY |
| NO: 31 | of HCV 1a | |
| | polyprotein | |
| SEQ ID | Sub-sequence | GEVQIVSTAAQTFLA |
| NO: 32 | of HCV 1a | |
| | polyprotein | |
| SEQ ID | Sub-sequence | QTFLATCINGVCWTV |
| NO: 33 | of HCV 1a | |
| | polyprotein | |
| SEQ ID | Sub-sequence | CINGVCWTVY |
| NO: 34 | of HCV 1a | |
| | polyprotein | |
| SEQ ID | Sub-sequence | SSDLYLVTRHADVIP |
| NO: 35 | of HCV 1a | |
| | polyprotein | |
| SEQ ID | Sub-sequence | YLVTRHAD |
| NO: 36 | of HCV 1a | |
| | polyprotein | |
| SEQ ID | Sub-sequence | LLCPAGHAV |
| NO: 37 | of HCV 1a | |
| | polyprotein | |
| SEQ ID | Sub-sequence | AVDFIPVEGLETTMR |
| NO: 38 | of HCV 1a | |
| | polyprotein | |
| SEQ ID | Sub-sequence | KIDTKYIMTCMSADL |
| NO: 39 | of HCV 1a | |
| | polyprotein | |
| SEQ ID | Degradation | PITKIDTKYIMTCMSADLEVVTSTWVLVGGVLAALA |
| NO: 40 | sequences | AYCLST |
| SEQ ID | Targeting | KKKRK |
| NO: 41 | sequence | |
| SEQ ID | Targeting | MLRT S SLFTRRVQP SLFRNILRLQ ST |
| NO: 42 | sequence | |
| SEQ ID | Targeting | KDEL |
| NO: 43 | sequence | |
| SEQ ID | C-terminal | DEMEECSQHLPGAGSSGDIMDYKDDDDKGSSGTGS |
| NO: 44 | degradation | GSGTSAPITAYAQQTRGLLGCIITSLTGRDKNQVE |
| | signal with | GEVQIVSTATQTFLATCINGVCWAVYHGAGTRTIA |
| | NS4A/4B | SPKGPVIQMYTNVDQDLV |
| | protease | GWPAPQGSRSLTPCTCGSSDLYLVTRHADVIPVRR |
| | cleavage site | RGDSRGSLLSPRPISYLKGSSGGPLLCPAGHAVGL |
| | | FRAAVCTRGVAKAVDFIPVENLETTMRSPVFTDNS |
| | | SPPAVTLTHPITKIDTKYIMTCMSADLEWTSTWVL |
| | | VGGVLAALAAYCLSTGCWIVGRIVLSGKPAIIPDR |
| | | EVLY |
| SEQ ID | N-terminal | MDYKDDDDKGSSGTGSGSGTSAPITAYAQQTRGLL |
| NO: 45 | degradation | GCIITSLTGRDKNQVEGEVQIVSTATQTFLATCIN |
| | signal with | GVCWAVYHGAGTRTIASPKGPVIQMYTNVDQDLVG |
| | HCV | WPAPQGSRSLTPCTCGSSDLYLVTRHADVIPVRRR |
| | NS5A/5B | GDSRGSLLSPRPISYLKGSSGGPLLCPAGHAVGLF |
| | protease | RAAVCTRGVAKAVDFIPVENLETTMRSPVFTDNSS |
| | cleavage site | PPAVTLTHPITKIDTKYIMTCMSADLEWTSTWVLV |
| | | GGVLAALAAYCLSTGCWIVGRIVLSGKPAGS |
| | | SGSSIIPDREVLYQEFEDWPCSMG |
| SEQ ID | PEST, Two | LQMLPESEDEESYDTESEFTEFTEDELPYDDGSLQ |
| NO: 46 | copies of | MLPESEDEESYDTESEFTEFTEDELPYDD |
| | residues 277- | |
| | 307 of IκBα | |
| | (human) | |
| SEQ ID | GRR, | EIKDKEEVQRKRQKLMPNFSDSFGGGSGAGAGGGG |
| NO: 47 | Residues | MFGSGGGGGGTGSTGPGYSFPH |
| | 352-408 of | |
| | p105 (human) | |
| SEQ ID | DRR, | IDDENGSVILQDDDYDDGNNHIPFEDDDVYNYNDN |
| NO: 48 | Residue 210- | DDDDERIEFEDDDDDDDDSIDNDSVMDRKQPHKAE |
| | 295 of Cdc34 | DESEDVEDVERVSKKD |
| | (yeast)) | |
| SEQ ID | SNS, Tandem | PESMREEYRKEGSKRIKCPDCEPFCNKRGSPESMR |
| NO: 49 | repeat of SP2 | EEYRKE |
| | and NB (SP2- | |
| | NB-SP2) | |
| SEQ ID | RPB, (Four | RSYSPTSPNYSPTSPSGSYSPTSPNYSPTSPSGGS |
| NO: 50 | copies of | RSYSPTSPNYSPTSPSGSYSPTSPNYSPTSPSG |
| | residues | |
| | 1688-1702 of | |
| | RPB1 (yeast) | |
| SEQ ID | SPmix, | PESMREEYRKEGSSLLTEVETPGSPESMREEYRKE |
| NO: 51 | Tandem | GSSLLTEVETPGSPESMREEYRKE |
| | repeat of SP1 | |
| | and SP2 | |
| | (SP2-SP1- | |
| | SP2-SP1- | |
| | SP2) | |
| | (Influenza A | |
| | virus M2 | |
| | protein) | |
| SEQ ID | Three copies | LIEEVRHRLKTTENSGSLIEEVRHRLKTTENSGSL |
| NO: 52 | of residue 79- | IEEVRHRLKTTENSGS |
| | 93 of | |
| | Influenza A | |
| | virus NS | |
| | protein | |
| SEQ ID | Residue 106- | FPPEVEEQDDGTLPMSCAQESGMDRHPAACASARI |
| NO: 53 | 142 of | NV |
| | ornithine | |
| | decarboxylase | |
| SEQ ID | mODC DA, | SHGFPPEVEEQAAGTLPMSCAQESGMDRHPAACAS |
| NO: 54 | amino acids | ARINV |
| | 422-461 of | |
| | mODC | |
| | (D433A, | |
| | D434A) | |

What is claimed is:

1. A fusion protein, comprising:
a polypeptide of interest;
a variant hepatitis C virus (HCV) nonstructural protein 3 (NS3) protease; and
a cognate protease cleavage site,
wherein the variant HCV NS3 protease comprises one or more mutations; and
wherein the one or more mutations decrease immunogenicity when the fusion protein is expressed in a mammalian cell.

2. The fusion protein of claim 1, wherein the variant HCV NS3 protease is derived from an HCV polyprotein comprising the amino acid sequence of SEQ ID NO: 1.

3. The fusion protein of claim 1 or claim 2, wherein the one or more mutations comprise one or more amino acid substitutions.

4. The fusion protein of claim 3, wherein the one or more amino acid substitutions correspond to amino acid substitutions within SEQ ID NO: 1.

5. The fusion protein of claim 4, wherein the one or more amino acid substitutions are at one or more positions corresponding to positions 1038 to 1047 of SEQ ID NO: 1, positions 1057 to 1081 of SEQ ID NO: 1, positions 1073 to 1081 of SEQ ID NO: 1, positions 1073 to 1082 of SEQ ID NO: 1, positions 1127 to 1141 of SEQ ID NO: 1, positions 1131 to 1138 of SEQ ID NO: 1, positions 1169 to 1177 of SEQ ID NO: 1, and/or positions 1192 to 1206 of SEQ ID NO: 1.

6. The fusion protein of claim 5, wherein the one or more amino acid substitutions are selected from the group consisting of a position corresponding to position 1062 of SEQ ID NO: 1, a position corresponding to position 1069 of SEQ ID NO: 1, a position corresponding to position 1070 of SEQ ID NO: 1, a position corresponding to position 1071 of SEQ ID NO: 1, a position corresponding to position 1072 of SEQ ID NO: 1, a position corresponding to position 1074 of SEQ ID NO: 1, a position corresponding to position 1075 of SEQ ID NO: 1, a position corresponding to position 1077 of SEQ ID NO: 1, a position corresponding to position 1078 of SEQ ID NO: 1, a position corresponding to position 1079 of SEQ ID NO: 1, a position corresponding to position 1080 of SEQ ID NO: 1, a position corresponding to position 1031 of SEQ ID NO: 1, a position corresponding to position 1132 of SEQ ID NO: 1, a position corresponding to position 1133 of SEQ ID NO: 1, a position corresponding to position 1195 of SEQ ID NO: 1, a position corresponding to position 1196 of SEQ ID NO: 1, a position corresponding to position 1201 of SEQ ID NO: 1, a position corresponding to position 1202 of SEQ ID NO: 1, and any combination thereof.

7. The fusion protein of claim 5, wherein the one or more amino acid substitutions are selected from the group consisting of an Ile to Leu substitution at a position corresponding to position 1074 of SEQ ID NO: 1, an Ile to Met substitution at a position corresponding to position 1074 of SEQ ID NO: 1, an Asn to Ala substitution at a position corresponding to position 1075 of SEQ ID NO: 1, a Val to Ala substitution at a position corresponding to position 1077 of SEQ ID NO: 1, a Cys to Phe substitution at a position corresponding to position 1078 of SEQ ID NO: 1, a Trp to Ala substitution at a position corresponding to position 1079 of SEQ ID NO: 1, a Thr to Ala substitution at a position corresponding to position 1080 of SEQ ID NO: 1, a Val to Ala substitution at a position corresponding to position 1081 of SEQ ID NO: 1, a Val to Asn substitution at a position corresponding to position 1081 of SEQ ID NO: 1, and any combination thereof.

8. The fusion protein of claim 5, wherein the one or more amino acid substitutions comprise a Thr to Ala substitution at a position corresponding to position 1080 of SEQ ID NO: 1.

9. The fusion protein of claim 5, wherein the one or more amino acid substitutions comprise a Thr to Ala substitution at a position corresponding to position 1080 of SEQ ID NO: 1 and a Val to Ala substitution at a position corresponding to position 1077 of SEQ ID NO: 1.

10. The fusion protein of claim 5, wherein the one or more amino acid substitutions comprise a Thr to Ala substitution at a position corresponding to position 1080 of SEQ ID NO: 1 and a Val to Ala substitution at a position corresponding to position 1081 of SEQ ID NO: 1.

11. The fusion protein of any one of claims 1-10, further comprising an HCV NS4A co-factor.

12. The fusion protein of any one of claims 1-11, further comprising a degron, wherein the degron is operably linked to the polypeptide of interest.

13. The fusion protein of claim 12, wherein the degron is selected from the group consisting of HCV NS4 degron, PEST (two copies of residues 277-307 of human IκBα) (SEQ ID NO: 46), GRR (residues 352-408 of human p105) (SEQ ID NO: 47), DRR (residues 210-295 of yeast Cdc34) (SEQ ID NO: 48), SNS (tandem repeat of SP2 and NB (SP2-NB-SP2 of influenza A or influenza B) (SEQ ID NO: 49), RPB (four copies of residues 1688-1702 of yeast RPB) (SEQ ID NO: 50), SPmix (tandem repeat of SP1 and SP2 (SP2-SP1-SP2-SP1-SP2 of influenza A virus M2 protein) (SEQ ID NO: 51), NS2 (three copies of residues 79-93 of influenza A virus NS protein) (SEQ ID NO: 52), ODC (residues 106-142 of ornithine decarboxylase) (SEQ ID NO: 53), Nek2A, mouse ODC (residues 422-461), mouse ODC_DA (residues 422-461 of mODC including D433A and D434A point mutations) (SEQ ID NO: 54), an APC/C degron, a COP1 E3 ligase binding degron motif, a CRL4-Cdt2 binding PIP degron, an actinfilin-binding degron, a KEAP1 binding degron, a KLHL2 and KLHL3 binding degron, an MDM2 binding motif, an N-degron, a hydroxyproline modification in hypoxia signaling, a phytohormone-dependent SCF-LRR-binding degron, an SCF ubiquitin ligase binding phosphodegron, a phytohormone-dependent SCF-LRR-binding degron, a DSGxxS (SEQ ID NO: 55) phospho-dependent degron, an Siah binding motif, an SPOP SBC docking motif, and a PCNA binding PIP box.

14. The fusion protein of any one of claims 1-13, wherein the variant HCV NS3 protease comprises one or more additional mutations.

15. The fusion protein of claim 14, wherein the one or more additional mutations modulate enzymatic activity of the variant HCV NS3 protease.

16. The fusion protein of claim 14 or claim 15, wherein the one or more additional mutations are one or more additional amino acid substitutions.

17. The fusion protein of claim 16, wherein the one or more additional amino acid substitutions are at one or more positions corresponding to position 1074 of SEQ ID NO: 1, position 1078 of SEQ ID NO: 1, and/or position 1079 of SEQ ID NO: 1.

18. The fusion protein of claim 17, wherein the one or more additional amino acid substitutions are selected from the group consisting of an Ile to Ala substitution at a position corresponding to position 1074 of SEQ ID NO: 1, a Trp to Ala substitution at a position corresponding to position 1079 of SEQ ID NO: 1, and any combination thereof.

19. The fusion protein of claim 18, wherein the one or more additional amino acid substitutions decrease enzymatic activity of the variant HCV NS3 protease.

20. The fusion protein of claim 17, wherein the one or more additional amino acid substitutions comprise a Cys to Ala substitution at a position corresponding to position 1078 of SEQ ID NO: 1.

21. The fusion protein of claim 20, wherein the one or more additional amino acid substitutions increase enzymatic activity of the variant HCV NS3 protease.

22. The fusion protein of any one of claims 1-21, wherein the cognate protease cleavage site comprises an amino acid sequence selected from the group consisting of any of the amino acid sequences listed in Table 1.

23. The fusion protein of any one of claims 1-21, wherein the cognate protease cleavage site comprises an amino acid sequence selected from the group consisting of CMSADLEVVTSTWVLVGGVL (SEQ ID NO: 4), YQEFDEMEECSQHLPYIEQG (SEQ ID NO. 5), WISSECTTPCSGSWLRDIWD (SEQ ID NO: 6), and GADTEDVVCCSMSYSWTGAL (SEQ ID NO: 7).

24. The fusion protein of any one of claims 1-21, wherein the cognate protease cleavage site comprises an amino acid sequence selected from the group consisting of ADLEVVTSTWL (SEQ ID NO 8), DEMEECSQHL (SEQ ID NO: 9), ECTTPCSGSWL (SEQ ID NO: 10), and EDVVPCSMG (SEQ ID NO: 11).

25. The fusion protein of any one of claims 22-24, wherein the cognate protease cleavage site comprises one or more mutations.

26. The fusion protein of claim 25, wherein the one or more mutations comprise one or more amino acid substitutions.

27. The fusion protein of claim 25 or claim 26, wherein the one or more mutations increase the catalytic rate of cleavage.

28. The fusion protein of claim 25 or claim 26, wherein the one or more mutations decrease the catalytic rate of cleavage.

29. The fusion protein of any one of claims 1-28, wherein the polypeptide of interest is selected from the group consisting of a membrane protein, a receptor, a hormone, a cytokine, a transport protein, a transcription factor, a cytoskeletal protein, an extracellular matrix protein, a signal-transduction protein, and an enzyme.

30. The fusion protein of any one of claims 1-28, wherein the polypeptide of interest comprises a biologically active domain of a protein.

31. The fusion protein of claim 30, wherein the biologically active domain is a catalytic domain, a ligand binding domain, or a protein-protein interaction domain.

32. The fusion protein of any one of claims 1-31, wherein the polypeptide of interest is a receptor selected from the group consisting of a T cell receptor (TCR), a chimeric T cell receptor, an artificial T cell receptor, a synthetic T cell receptor, a chimeric immunoreceptor, an antibody-coupled T cell receptor (ACTR), a T cell receptor fusion construct (TRUC), and a chimeric antigen receptor (CAR).

33. The fusion protein of any one of claims 1-31, wherein the polypeptide of interest is a chimeric antigen receptor (CAR).

34. The fusion protein of any one of claims 1-28, wherein the polypeptide of interest is a cytokine.

35. The fusion protein of claim 34, wherein the cytokine is a proinflammatory cytokine.

36. The fusion protein of any one of claims 1-35, wherein the cognate protease cleavage site is localized within a domain of the polypeptide of interest.

37. The fusion protein of any one of claims 1-35, wherein the polypeptide of interest comprises multiple domains.

38. The fusion protein of claim 37, wherein the cognate protease cleavage site is localized between the multiple domains of the polypeptide of interest.

39. The fusion protein of any one of claims 1-38, wherein the variant HCV NS3 protease can be repressed by a protease inhibitor.

40. The fusion protein of claim 39, wherein the protease inhibitor is selected from the group consisting of simeprevir, danoprevir, asunaprevir, ciluprevir, boceprevir, sovaprevir, paritaprevir, telaprevir, grazoprevir, glecaprevir, and voxiloprevir.

41. The fusion protein of any one of claims 1-40, further comprising a targeting sequence.

42. The fusion protein of claim 41, wherein the targeting sequence is selected from the group consisting of a secretory protein signal sequence, a membrane protein signal sequence, a nuclear localization sequence, a nucleolar localization signal sequence, an endoplasmic reticulum localization sequence, a peroxisome localization sequence, a mitochondrial localization sequence, and a protein binding motif sequence.

43. The fusion protein of any one of claims 1-42, wherein the variant NS3 protease is derived from an HCV NS3 protease having the amino acid sequence of SEQ ID NO: 2.

44. A polynucleotide encoding the fusion protein of any one of claims 1-43.

45. A vector comprising the polynucleotide of claim 44.

46. A cell comprising the fusion protein of any one of claims 1-43, the polynucleotide of claim 44, or the vector of claim 45.

47. The cell of claim 46, wherein the cell is an immune cell or a cell line derived from an immune cell.

48. The cell of claim 47, wherein the immune cell is selected from the group consisting of a T cell, a B cell, an NK cell, an NKT cell, an innate lymphoid cell, a mast cell, an eosinophil, a basophils, a macrophage, a neutrophil, a dendritic cell, and any combinations thereof.

49. The cell of claim 46, wherein the cell is a mesenchymal stromal cell.

50. A pharmaceutical composition comprising the fusion protein of any one of claims 1-43 and an excipient.

51. A pharmaceutical composition comprising the cell of any one of claims 46-49 and an excipient.

52. A method of treating a subject in need thereof, comprising administering the pharmaceutical composition of claim 50 or claim 51.

53. A method of regulating activity of a protein of interest, comprising:
a) providing a population of cells comprising the fusion protein of any one of claims 1-43, the polynucleotide of claim 44, or the vector of claim 45; and
b) contacting the population of cells with a protease inhibitor.

54. The method of claim 53, further comprising the step of removing the protease inhibitor from the population of cells.

55. The method of claim 53 or claim 54, further comprising the step of administering the population of cells to a subject in need of a cell-based therapy.

56. A method of treating a subject in need of a cell-based therapy, comprising administering to the subject a population of cells comprising the fusion protein of any one of claims 1-43, the polynucleotide of claim 44, or the vector of claim 45.

57. The method of claim 56, wherein the population of cells was cultured in the presence of a protease inhibitor capable of inhibiting the repressible protease.

58. The method of claim 56, wherein the population of cells was cultured in the absence of a protease inhibitor capable of inhibiting the repressible protease.

59. The method of any one of claims 56-58, further comprising the step of administering to the subject the protease inhibitor capable of inhibiting the repressible protease.

60. The method of claim 59, further comprising the step of withdrawing the protease inhibitor capable of inhibiting the repressible protease from the subject.
